SEQUENCE LISTING (30 sequences)

SEQ ID NO: 1 - DNA, 1750 bp, Papaver somniferum
atagaacaaattgacggaattgtacttccatatatcagcagcatttgcaaactaaactctctctctctcgttctcctttgcagtcatggcgggtctacttgtaccctcctcttcagctctcttaccttctcgattcccctcctcatcttcttcctcaaaactatctcctttagtttcttctatatcctcaacttcaggtacaaacattctactcttcttttactactacatggtttttcattgtttgagtttcttacttcaatttttatgtttataatcacaaatttagttttgtttgattatatgtacaggccagatttatgggagaaagttcaatggaggaggaattggaaaccttattaaaaagggaagatctcaatttcatttctcagtgaaaaatgttgctactgaaatcattcctgcagaggtttgaaaccctttacctactatgctaccttgattaaattagccttttatgtatagttgtttttgttatgtgtacttttaattagcccatttctactatgctattagaattgaagggatgagaccatgaaattttcaggctaaaaaacgtgctaaggaaagtcagagaccagtgtatccttttgcagcaatagtaggacaagatgaaatgaaattgtgtctactgcttaatgttattgacccaaaaattggaggtgttatgataatgggtgatagaggaactggaaaatcaacaactgttaggtctctagttgatttactacctgaaatcaaagtggtagcaggggatccattcaattctgacccggaagatcccgaatccatgggtgtggaagtcagagaaagtgtcttaaatggtgaggaactagagattgtgactactaaaatcaacatggttgatcttccactaggtgctaccgaagatagggtttgtggtacaattgatattgagaaagctttaacagaaggagtgaaggcattcgagccaggtctacttgctaaagcgaatagaggaattctatatgtggatgaagtaaatcttttggatgatcatttggttgatgttcttttagattctgctgcctctgggtggaacactgtcgagagagagggaatctcaatttctcatcctgctcggtttatcctaattggttcaggaaatccagaagaaggtgagcttagaccacaacttctcgaccgatttgggatgcatgcacaagttgggactgtgagagatgcggagcttagggtgaagattgtagaggagagggcacggtttgaccagaaccctaaagagttccgtactacctacaatgtagaccaagataaactccaagaacagataactgctgcaaggaaaattctttcttctgtcaaggttgatcgcgatctttgtgtgaagatatctaaagtttgtgcagaactaaatgttgatggattgagaggagatattgttactaacagggcagcaagagctttagctgctctgaagggaagagatcaagtaactccggaggatattgcaactgtcattcccaattgtttaagacatcgtcttagaaaggatccattagaatcaatagactctggattactcgtcattgaaaaattctacgaggtctttggttgaaggaggtaaataaccgctatgttcttcaatttttgttcgaatgtttgttggttaatcttcttttagacaacacccgctcttttgtgttattttccttctttcattgctcgtgttctcttaatcctagtttttcct

SEQ ID NO: 2 - DNA, 1278 bp, Papaver somniferum
atggcgggtctacttgtaccctcctcttcagctctcttaccttctcgattcccctcctcatcttcttcctcaaaactatctcctttagtttcttctatatcctcaacttcaggccagatttatgggagaaagttcaatggaggaggaattggaaaccttattaaaaagggaagatctcaatttcatttctcagtgaaaaatgttgctactgaaatcattcctgcagaggctaaaaaacgtgctaaggaaagtcagagaccagtgtatccttttgcagcaatagtaggacaagatgaaatgaaattgtgtctactgcttaatgttattgacccaaaaattggaggtgttatgataatgggtgatagaggaactggaaaatcaacaactgttaggtctctagttgatttactacctgaaatcaaagtggtagcaggggatccattcaattctgacccggaagatcccgaatccatgggtgtggaagtcagagaaagtgtcttaaatggtgaggaactagagattgtgactactaaaatcaacatggttgatcttccactaggtgctaccgaagatagggtttgtggtacaattgatattgagaaagctttaacagaaggagtgaaggcattcgagccaggtctacttgctaaagcgaatagaggaattctatatgtggatgaagtaaatcttttggatgatcatttggttgatgttcttttagattctgctgcctctgggtggaacactgtcgagagagagggaatctcaatttctcatcctgctcggtttatcctaattggttcaggaaatccagaagaaggtgagcttagaccacaacttctcgaccgatttgggatgcatgcacaagttgggactgtgagagatgcggagcttagggtgaagattgtagaggagagggcacggtttgaccagaaccctaaagagttccgtactacctacaatgtagaccaagataaactccaagaacagataactgctgcaaggaaaattctttcttctgtcaaggttgatcgcgatctttgtgtgaagatatctaaagtttgtgcagaactaaatgttgatggattgagaggagatattgttactaacagggcagcaagagctttagctgctctgaagggaagagatcaagtaactccggaggatattgcaactgtcattcccaattgtttaagacatcgtcttagaaaggatccattagaatcaatagactctggattactcgtcattgaaaaattctacgaggtctttggttga

SEQ ID NO: 3 - protein, 425 aa, Papaver somniferum
MAGLLVPSSSALLPSRFPSSSSSSKLSPLVSSISSTSGQIYGRKFNGGGIGNLIKKGRSQFHFSVKNVATEIIPAEAKKRAKESQRPVYPFAAIVGQDEMKLCLLLNVIDPKIGGVMIMGDRGTGKSTTVRSLVDLLPEIKVVAGDPFNSDPEDPESMGVEVRESVLNGEELEIVTTKINMVDLPLGATEDRVCGTIDIEKALTEGVKAFEPGLLAKANRGILYVDEVNLLDDHLVDVLLDSAASGWNTVEREGISISHPARFILIGSGNPEEGELRPQLLDRFGMHAQVGTVRDAELRVKIVEERARFDQNPKEFRTTYNVDQDKLQEQITAARKILSSVKVDRDLCVKISKVCAELNVDGLRGDIVTNRAARALAALKGRDQVTPEDIATVIPNCLRHRLRKDPLESIDSGLLVIEKFYEVFG

SEQ ID NO: 4 - DNA, 1715 bp, Papaver somniferum
tccatatatcagcagcatttgcaaactaaactctctctctctcgttctcctttgcagtcatggcgggtctacttgtaccctcctcttcagctctcttaccttctcgattcccctcctcatcttcttcctcaaaactatctcctttagtttcttctatatcctcaacttcaggtacaaacattctactcttcttttactactacatggtttttcattgtttgagtttcttacttcaatttttatgtttataatcacaaatttagttttgtttgattatatgtacaggccagatttatgggagaaagttcaatggaggaggaattggaaaccttattaaaaagggaagatctcaatttcatttctcagtgaaaaatgttgctactgaaatcattcctgcagaggtttgaaaccctttacctactatgctaccttgattaaattagccttttatgtatagttgtttttgttatgtgtacttttaattagcccatttctactatgctattagaattgaagggatgagaccatgaaattttcaggctaaaaaacgtgctaaggaaagtcagagaccagtgtatccttttgcagcaatagtaggacaagatgaaatgaaattgtgtctactgcttaatgttattgacccaaaaattggaggtgttatgataatgggtgatagaggaactggaaaatcaacaactgttaggtctctagttgatttactacctgaaatcaaagtggtagcaggggatccattcaattctgacccggaagatcccgaatccatgggtgtggaagtcagagaaagtgtcttaaatggtgaggaactagagattgtgactactaaaatcaacatggttgatcttccactaggtgctaccgaagatagggtttgtggtacaattgatattgagaaagctttaacagaaggagtgaaggcattcgagccaggtctacttgctaaagcgaatagaggaattctatatgtggatgaagtaaatcttttggatgatcatttggttgatgttcttttagattctgctgcctctgggtggaacactgtcgagagagagggaatctcaatttctcatcctgctcggtttatcctaattggttcaggaaatccagaagaaggtgagcttagaccacaacttctcgaccgatttgggatgcatgcacaagttgggactgtgagagatgcggagcttagggtgaagattgtagaggagagggcacggtttgaccagaaccctaaagagttccgtactacctacaatgtagaccaagataaactctaagaacagataactgctgcaaggaaaattctttcttctgtcaaggttgatcgcgatctttgtgtgaagatatctaaagtttgtgcagaactaaatgttgatggattgagaggagatattgttactaacagggcagcaagagctttagctgctctgaagggaagagatcaagtaactccggaggatattgcaactgtcattcccaattgtttaagacatcgtcttagaaaggatccattagaatcaatagactctggattactcgtcattgaaaaattctacgaggtctttggttgaaggaggtaaataagcgctatgttcttcaatttttgttcgaatgtttgttggttaatcttcttttagacaacacccgttcttttgtgttattttccttctttcatttctcatgttctcttaatccta

SEQ ID NO: 5 - DNA, 1530 bp, Papaver somniferum
atggcgggtctacttgtaccctcctcttcagctctcttaccttctcgattcccctcctcatcttcttcctcaaaactatctcctttagtttcttctatatccgcaacttcaggtacaaacattctactcttcttttactactacgtggtttttcattgtttgagtttcttacttcaatttttatgtttataatcacaaatttagttttgtttgattatatgtacaggccagatttatgggagaaagttcaatggaggaggaattggaaaccttattaaaaagggaagatctcaatttcatttctcagtgaaaaatgttgctactgaaatcattcctgcagaggtttgaaaccctttacctactatgctaccttgattaaattagccttttatgtatagttgtttttgttatgtgtacttttaattagcccatttctactatgctattagaattgaagggatgagaccatgaaattttcaggctaaaaaacgtgctaaggaaagtcagagaccagtgtatccttttgcagcaatagtaggacaagatgaaatgaaattgtgtctactgcttaatgttattgacccaaaaattggaggtgttatgataatgggtgatagaggaactggaaaatcaacaactgttaggtctctagttgatttactccctgaaatcaaagtggtagcaggggatccattcaattctgacccggaagatcccgaatccatgggtgtggaagtcagagaaagtgtcttaaatggtgaggaactagagattgtgactactaaaatcaacatggttgatcttccactaggtgctaccgaagatagggtttgtggtacaattgatattgagaaagctttaacagaaggagtgaaggcattcgagccaggtctacttgctaaagcgaatagaggaattctatatgtggatgaagtaaatcttttggatgatcatttggttgatgttcttttagattctgctgcctctgggtggaacactgtcgagagagagggaatctcaatttctcatcctgctcggtttatcctaattggttcaggaaatccagaagaaggtgagcttagaccacaacttctcgaccgatttgggatgcatgcacaagttgggactgtgagagatgcggagcttagggtgaagattgtagaggagagggcacggtttgaccagaaccctaaagagttccgtactacctacaatgtagaccaagataaactccaagaacagataactgctgcaaggaaaattctttcttctgtcaaggttgatcgcgatctttgtgtgaagatatctaaagtttgtgcagaactaaatgttgatggattgagaggagatattgttactaacagggcagcaagagctttagctgctctgaagggaagagatcaagtaactccggaggatattgcaactgtcattcccaattgtttaagacatcgtcttagaaaggatccattagaatcaatagactctggattactcgtcattgaaaaattctacgaggtctttggttga

SEQ ID NO: 6 - DNA, 1641 bp, Papaver somniferum
gcaactaaactctctctctctctcctttgcagtcatggcgggtctacttgtaccctcctcttcagctctcttaccttctcgattcccctcctcttcttcttcctcaaaactatctcctttagtttcttctatatcctcaacttcaggtacaaacattctactcttcttttactactacatgggttttcattgtttgagtttcttgcttcaatttttatgtttataattctcaatttagttttgtttgattttatgtacaggccaaatttatgggagaaagttcaatggaggaggaattggaaaccttattaaaaagggaagatctcaatttcatttctcagtgaaaaatgttgctactgaaatcaatcctgcagaggtttgaaaccctttacctactatgttaccttgattaaattagcctttcatgtgtagttgtttttgttatgtgtacttttaattagcccatttctgctatgctattagaattgaggggatgagaccatgaaattttcaggctaaaaaacgtgccaaggaaagtcagagaccagtgtatccttttgcagcaatagtaggacaagatgaaatgaaattgtgtctactgcttaatgttattgacccaaaaattggaggtgttatgataatgggtgatagaggaactggaaaatcaacaactgttaggtctctagttgatttactacctgaaatcaaagtggtagcaggggatccattcaattctgacccggaagatcctgaatccatgggtgtggaagtcagagaaagtgtcttaaatggtgagaaactagagattgtcacgactaaaatcaacatggttgatcttccactaggtgctactgaagatagggtttgtggtacaattgatatcgagaaagctctgacagaaggagtgaaggcattcgagccaggtctacttgctaaagctaatagaggaattctatacgtggatgaagtaaatcttttggatgatcatttggttgatgtgcttttagattctgctgcctctgggtggaacactgtcgagagagaaggaatctctatttctcatcctgctcgttttatcctaattggttcaggaaatccagaagaaggtgagcttagaccacaacttctcgaccgattcggaatgcatgcacaagttgggactgtgagagatgcggagcttagggtgaagattgtagaggagagggcacggtttgaccagaaccctaaagagtttcgtactacctacaatgtagaccaagataaactccaagaacagataactgctgcaaggaaaattctttcttctgtcaaggttgatcgcgatctttgtgtgaagatatctaaagtttgtgcagaactgaatgttgatggattgagaggagatattgttactaacagggcagcaagagctttagctgctctgaagggaagagatcgagtaactccggaggatattgcaactgtcattcccaattgtttaagacatcgtcttagaaaggatccattagaatcaatagactctggattactcgtcattgaaaaattctacgaggtctttggttgaaggaggtaaataagcgctatgttcttcaatttttgttcgaatgtttgttggttaatcttcttttagacaacacccgt

SEQ ID NO: 7 - DNA, 1278 bp, Papaver somniferum
atggcgggtctacttgtaccctcctcttcagctctcttaccttctcgattcccctcctcttcttcttcctcaaaactatctcctttagtttcttctatatcctcaacttcaggccaaatttatgggagaaagttcaatggaggaggaattggaaaccttattaaaaagggaagatctcaatttcatttctcagtgaaaaatgttgctactgaaatcaatcctgcagaggctaaaaaacgtgccaaggaaagtcagagaccagtgtatccttttgcagcaatagtaggacaagatgaaatgaaattgtgtctactgcttaatgttattgacccaaaaattggaggtgttatgataatgggtgatagaggaactggaaaatcaacaactgttaggtctctagttgatttactacctgaaatcaaagtggtagcaggggatccattcaattctgacccggaagatcctgaatccatgggtgtggaagtcagagaaagtgtcttaaatggtgagaaactagagattgtcacgactaaaatcaacatggttgatcttccactaggtgctactgaagatagggtttgtggtacaattgatatcgagaaagctctgacagaaggagtgaaggcattcgagccaggtctacttgctaaagctaatagaggaattctatacgtggatgaagtaaatcttttggatgatcatttggttgatgtgcttttagattctgctgcctctgggtggaacactgtcgagagagaaggaatctctatttctcatcctgctcgttttatcctaattggttcaggaaatccagaagaaggtgagcttagaccacaacttctcgaccgattcggaatgcatgcacaagttgggactgtgagagatgcggagcttagggtgaagattgtagaggagagggcacggtttgaccagaaccctaaagagtttcgtactacctacaatgtagaccaagataaactccaagaacagataactgctgcaaggaaaattctttcttctgtcaaggttgatcgcgatctttgtgtgaagatatctaaagtttgtgcagaactgaatgttgatggattgagaggagatattgttactaacagggcagcaagagctttagctgctctgaagggaagagatcgagtaactccggaggatattgcaactgtcattcccaattgtttaagacatcgtcttagaaaggatccattagaatcaatagactctggattactcgtcattgaaaaattctacgaggtctttggttga

SEQ ID NO: 8 - protein, 425 aa, Papaver somniferum
MAGLLVPSSSALLPSRFPSSSSSSKLSPLVSSISSTSGQIYGRKFNGGGIGNLIKKGRSQFHFSVKNVATEINPAEAKKRAKESQRPVYPFAAIVGQDEMKLCLLLNVIDPKIGGVMIMGDRGTGKSTTVRSLVDLLPEIKVVAGDPFNSDPEDPESMGVEVRESVLNGEKLEIVTTKINMVDLPLGATEDRVCGTIDIEKALTEGVKAFEPGLLAKANRGILYVDEVNLLDDHLVDVLLDSAASGWNTVEREGISISHPARFILIGSGNPEEGELRPQLLDRFGMHAQVGTVRDAELRVKIVEERARFDQNPKEFRTTYNVDQDKLQEQITAARKILSSVKVDRDLCVKISKVCAELNVDGLRGDIVTNRAARALAALKGRDRVTPEDIATVIPNCLRHRLRKDPLESIDSGLLVIEKFYEVFG

SEQ ID NO: 9 - DNA, 1530 bp, Papaver somniferum
atggcgggtctacttgtaccctcctcttcagctctcttaccttctcgattcccctcctcttcttcttcctcaaaactatctcctttagtttcttctatatcctcaacttcaggtacaaacattctactcttcttttactactacatgggttttcattgtttgagtttcttgcttcaatttttatgtttataattctcaatttagttttgtttgattttatgtacaggccaaatttatgggagaaagttcaatggaggaggaattggaaaccttattaaaaagggaagatctcaatttcatttctcagtgaaaaatgttgctactgaaatcaatcctgcagaggtttgaaaccctttacctactatgttaccttgattaaattagcctttcatgtgtagttgtttttgttatgtgtacttttaattagcccatttctgctatgctattagaattgaggggatgagaccatgaaattttcaggctaaaaaacgtgccaaggaaagtcagagaccagtgtatccttttgcagcaatagtaggacaagatgaaatgaaattgtgtctactgcttaatgttattggcccaaaaattggaggtgttatgataatgggtgatagaggaactggaaaatcaacaactgttaggtctctagttgatttactacctgaaatcaaagtggtagcaggggatccattcaattctgacccggaagatcctgaatccatgggtgtggaagtcagagaaagtgtcttaaatggtgagaaactagagattgtcacgactaaaatcaacatggttgatcttccactaggtgctactgaagatagggtttgtggtacaattgatatcgagaaagctctgacagaaggagtgaaggcattcgagccaggtctacttgctaaagctaatagaggaattctatacgtggatgaagtaaatcttttggatgatcatttggttgatgtgcttttagattctgctgcctctgggtggaacactgtcgagagagaaggaatctctatttctcatcctgctcgttttatcctaattggttcaggaaatccagaagaaggtgagcttagaccacaacttctcgaccgattcggaatgcatgcacaagttgggactgtgagagatgcggagcttagggtgaagattgtagaggagagggcacggtttgaccagaaccctaaagagtttcgtactacctacaatgtagaccaagataaactccaagaacagataactgctgcaaggaaaattctttcttctgtcaaggttgatcgcgatctttgtgtgaagatatctaaagtttgtgcagaactgaatgttgatggattgagaggagatattgttactaacagggcagcaagagctttagctgctctgaagggaagagatcgagtaactccggaggatattgcaactgtcattcccaattgtttaagacatcgtcttagaaaggatccattagaatcaatagactctggattactcgtcattgaaaaattctacgaggtctttggttga

SEQ ID NO: 10 - protein, 425 aa, Papaver somniferum
MAGLLVPSSSALLPSRFPSSSSSSKLSPLVSSISSTSGQIYGRKFNGGGIGNLIKKGRSQFHFSVKNVATEINPAEAKKRAKESQRPVYPFAAIVGQDEMKLCLLLNVIGPKIGGVMIMGDRGTGKSTTVRSLVDLLPEIKVVAGDPFNSDPEDPESMGVEVRESVLNGEKLEIVTTKINMVDLPLGATEDRVCGTIDIEKALTEGVKAFEPGLLAKANRGILYVDEVNLLDDHLVDVLLDSAASGWNTVEREGISISHPARFILIGSGNPEEGELRPQLLDRFGMHAQVGTVRDAELRVKIVEERARFDQNPKEFRTTYNVDQDKLQEQITAARKILSSVKVDRDLCVKISKVCAELNVDGLRGDIVTNRAARALAALKGRDRVTPEDIATVIPNCLRHRLRKDPLESIDSGLLVIEKFYEVFG

SEQ ID NO: 11 - DNA, 1530 bp, Papaver somniferum
atggcgggtctacttgtaccctcctcttcagctctcttaccttctcgattcccctcctcttcttcttcctcaaaactatctcctttagtttcttctatatcctcaacttcaggtacaaacattctactcttcttttactactacatgggttttcattgtttgagtttcttgcttcaatttttatgtttataattctcaatttagttttgtttgattttatgtacaggccaaatttatgggagaaagttcaatggaggaggaattggaaaccttattaaaaagggaagatctcaatttcatttctcagtgaaaaatgttgctactgaaatcaatcctgcagaggtttgaaaccctttacctactatgttaccttgattaaattagcctttcatgtgtagttgtttttgttatgtgtacttttaattagcccatttctgctatgctattagaattgaggggatgagaccatgaaattttcaggctaaaaaacgtgccaaggaaagtcagagaccagtgtatccttttgcagcaatagtaggacaagatgaaatgaaattgtgtctactgcttaatgttattgacccaaaaattggaggtgttatgataatgggtgatagaggaactggaaaatcaacaactgttaggtctctagttgatttactacctgaaatcaaagtggtagcaggggatccattcaattctgacccggaagatcctgaatccatgggtgtggaagtcagagaaagtgtcttaaatggtgagaaactagagattgtcacgactaaaatcaacatggttgatcttccactaggtgctactgaagatagggtttgtggtacaattgatatcgagaaagctctgacagaaggagtgaaggcattcgagccaggtctacttgctaaagctaatagaggaattctatacgtggatgaagtaaatcttttggatgatcatttgggtgaagggcttttagattctgctgcctctgggtggaacactgtcgagagagaaggaatctctatttctcatcctgctcgttttatcctaattggttcaggaaatccagaagaaggtgagcttagaccacaacttctcgaccgattcggaatgcatgcacaagttgggactgtgagagatgcggagcttagggtgaagattgtagaggagagggcacggtttgaccagaaccctaaagagtttcgtactacctacaatgtagaccaagataaactccaagaacagataactgctgcaaggaaaattctttcttctgtcaaggttgatcgcgatctttgtgtgaagatatctaaagtttgtgcagaactgaatgttgatggattgagaggagatattgttactaacagggcagcaagagctttagctgctctgaagggaagagatcgagtaactccggaggatattgcaactgtcattcccaattgtttaagacatcgtcttagaaaggatccattagaatcaatagactctggattactcgtcattgaaaaattctacgaggtctttggttga

SEQ ID NO: 12 - protein, 425 aa, Papaver somniferum
MAGLLVPSSSALLPSRFPSSSSSSKLSPLVSSISSTSGQIYGRKFNGGGIGNLIKKGRSQFHFSVKNVATEINPAEAKKRAKESQRPVYPFAAIVGQDEMKLCLLLNVIDPKIGGVMIMGDRGTGKSTTVRSLVDLLPEIKVVAGDPFNSDPEDPESMGVEVRESVLNGEKLEIVTTKINMVDLPLGATEDRVCGTIDIEKALTEGVKAFEPGLLAKANRGILYVDEVNLLDDHLGEGLLDSAASGWNTVEREGISISHPARFILIGSGNPEEGELRPQLLDRFGMHAQVGTVRDAELRVKIVEERARFDQNPKEFRTTYNVDQDKLQEQITAARKILSSVKVDRDLCVKISKVCAELNVDGLRGDIVTNRAARALAALKGRDRVTPEDIATVIPNCLRHRLRKDPLESIDSGLLVIEKFYEVFG

SEQ ID NO: 13 - DNA, 29 bp, Artificial Sequence
  Forward primer
atagaacaaattgacggaattgtacttcc

SEQ ID NO: 14 - DNA, 30 bp, Artificial Sequence
  Reverse primer
aggaaaaactaggattaagagaacacgagc

SEQ ID NO: 15 - DNA, 29 bp, Artificial Sequence
  Forward primer
gcaaaagattgacggaattgtacttcatc

SEQ ID NO: 16 - DNA, 31 bp, Artificial Sequence
  Reverse primer
caggaaaaactaggattaagagaacatgaga

SEQ ID NO: 17 - DNA, 32 bp, Artificial Sequence
  Forward primer
gcaaaatctgttaacagcaaaagatagaacaa

SEQ ID NO: 18 - DNA, 30 bp, Artificial Sequence
  Reverse primer
cctcgtgattagagtttgtttctgaattca

SEQ ID NO: 19 - DNA, 29 bp, Artificial Sequence
  Forward primer
gcaaaagattgacggaattgtacttcatc

SEQ ID NO: 20 - DNA, 31 bp, Artificial Sequence; identical to SEQ ID NO: 16
  Reverse primer

SEQ ID NO: 21 - DNA, 25 bp, Artificial Sequence
  Forward primer
acggtttgaccagaaccctaaagag

SEQ ID NO: 22 - DNA, 22 bp, Artificial Sequence
  Reverse primer
tcgcgatcaaccttgacagaag

SEQ ID NO: 23 - DNA, 22 bp, Artificial Sequence
  Probe 1 (mutant ′T′ SNP)
accaagataaactctaagaaca

SEQ ID NO: 24 - DNA, 21 bp, Artificial Sequence
  Probe 2 (WT ′C′ SNP)
accaagataaactccaagaac

SEQ ID NO: 25 - DNA, 20 bp, Artificial Sequence
  Forward primer
cggtttgaccagaaccctaa

SEQ ID NO: 26 - DNA, 21 bp, Artificial Sequence
  Reverse primer
aaagaattttccttgcagcag

SEQ ID NO: 27 - protein, 424 aa, Arabidopsis thaliana
MASLLGTSSSAIWASPSLSSPSSKPSSSPICFRPGKLFGSKLNAGIQIRPKKNRSRYHVSVMNVATEINSTEQVVGKFDSKKSARPVYPFAAIVGQDEMKLCLLLNVIDPKIGGVMIMGDRGTGKSTTVRSLVDLLPEINVVAGDPYNSDPIDPEFMGVEVRERVEKGEQVPVIATKINMVDLPLGATEDRVCGTIDIEKALTEGVKAFEPGLLAKANRGILYVDEVNLLDDHLVDVLLDSAASGWNTVEREGISISHPARFILIGSGNPEEGELRPQLLDRFGMHAQVGTVRDADLRVKIVEERARFDSNPKDFRDTYKTEQDKLQDQISTARANLSSVQIDRELKVKISRVCSELNVDGLRGDIVTNRAAKALAALKGKDRVTPDDVATVIPNCLRHRLRKDPLESIDSGVLVSEKFAEIFS

SEQ ID NO: 28 - protein, 418 aa, Arabidopsis thaliana
MASLLGRSPSSILTCPRISSPSSTSSMSHLCFGPEKLSGRIQFNPKKNRSRYHVSVMNVATEINSVEQAKKIDSKESARPVYPFAAIVGQDEMKLCLLLNVIDPKIGGVMIMGDRGTGKSTTVRSLVDLLPEITVVSGDPYNSDPRDPECMGKEVREKVQKGEELSVIETKINMVDLPLGATEDRVCGTIDIEKALTEGVKAFEPGLLAKANRGILYVDEVNLLDDHLVDVLLDSAASGWNTVEREGISISHPARFILIGSGNPEEGELRPQLLDRFGMHAQVGTVRDAELRVKIVEERARFDSNPKEFRETYQEEQLKLQEQITTARSNLSAVQIDQDLKVKISKVCAELDVDGLRGDMVINRAARALAALQGRDQVTAEDVGIVIPNCLRHRLRKDPLESMDSGILVTEKFYEVFT

SEQ ID NO: 29 - protein, 422 aa, Pisum sativum
MASTTLGTSSIAFLPSRYLSSPSSNPSIHTLSLSSGQSCGRKFCGGIGINGTKGKSRFPVVNVATEVNSVEQGLNTIAKESQRPVYPFAAIVGQDEMKLCLLLNVIDPKIGGVMIMGDRGTGKSTTVRSLVDLLPEIKVVFGDPYNSDPEDPEVMGIEVRDRVIKGEQLSIVLSKINMVDLPLGATEDRVCGTIDIEKALTEGVKAFEPGLLAKANRGILYVDEVNLLDDHLVDVLLDSAASGWNTVEREGISISHPARFILIGSGNPEEGELRPQLLDRFGMHAQVGTVRDAELRVKIVEERARFDKNPKEFRETYNAEQEKLTEQITSARSLLSSVQIDQDLKVKISRVCAELNVDGLRGDIVSNRAAKALAALKGRDKVSVEDIATVIPNCLRHRLRKDPLESIDSGLLVTEKFYEIFS

SEQ ID NO: 30 - protein, 421 aa, Glycine max
MASALGTSSIAVLPSRYFSSSSSKPSIHTLSLTSGQNYGRKFYGGIGIHGIKGRAQLSVTNVATEVNSVEQAQSIASKESQRPVYPFSAIVGQDEMKLCLLLNVIDPKIGGVMIMGDRGTGKSTTVRSLVDLLPEIKVVAGDPYNSDPQDPEFMGVEVRERVLQGEELSVVLTKINMVDLPLGATEDRVCGTIDIEKALTEGVKAFEPGLLAKANRGILYVDEVNLLDDHLVDVLLDSAASGWNTVEREGISISHPARFILIGSGNPEEGELRPQLLDRFGMHAQVGTVRDAELRVKIVEERGRFDKNPKEFRDSYKAEQEKLQQQITSARSVLSSVQIDQDLKVKISKVCAELNVDGLRGDIVTNRAAKALAALKGRDNVSAEDIATVIPNCLRHRLRKDPLESIDSGLLVTEKFYEVFS

(19) United States
(12) Patent Application Publication
(10) Pub. No.: US 2021/0000061 A1
(43) Pub. Date: Jan. 7, 2021

(54) A Plant of Papaver Somniferum With An Altered Alkaloid Profile

(71) Applicant: Tasmanian Alkaloids Pty Ltd, Westbury, Tasmania (AU)

(72) Inventors: Gregory Michael Symons, Turners Marsh, Tasmania (AU); Corey James Hudson, South Launceston, Tasmania (AU)

(21) Appl. No.: 16/977,315

(22) PCT Filed: Mar. 4, 2019
(86) PCT No.: PCT/AU2019/050179

§ 371 Date: Sep. 1, 2020

(30) Foreign Application Priority Data
Mar. 2, 2018 (AU) 2018900692

(51) Int. Cl. A01H 6/64; A01H 1/04; A01H 5/02; A01H 5/12; A61K 36/66; C12N 9/00; C12Q 1/6876

(52) U.S. Cl. CPC A01H 6/64; A01H 1/04; A01H 5/02; A01H 5/12; C12Q 2600/156; C12N 9/93; C12Q 1/6876; C12Y 606/01001; C12Q 2600/13; A61K 36/66

(57) ABSTRACT

There are described plants of Papaver somniferum having reduced expression or activity of magnesium chelatase where-by the plants yield poppy straw having an altered alkaloid profile. In embodiments of the plants the reduced expression or activity of magnesium chelatase is associated with a trait for a lightened leaf colour. There are also described methods for producing the plants, and poppy straw, latex, concentrate of poppy straw and opium from the plants.

### FIELD OF THE INVENTION

The present invention relates to plants of Papaver somniferum for the production of alkaloids, and to poppy straw and latex from the plants.

### BACKGROUND OF THE INVENTION

The morphinan alkaloids are an important subclass of benzylisoquinoline alkaloids which accumulate in the poppy capsules of the opium poppy Papaver somniferum, and include morphine, oripavine, codeine and thebaine amongst their number. Whilst morphine has traditionally been the major alkaloid in poppy straw and latex obtained from the capsules of P. somniferum, there has been significant research effort in modifying the morphine biosynthesis pathway and in recent times P. somniferum which accumulate thebaine or codeine as the predominant alkaloid have been grown on a commercial scale (see e.g., WO 98/02033, WO 2009/109012 and WO 2009/143574, all in the name of Tasmanian Alkaloids Pty Ltd, Westbury, Tasmania, Australia). P. somniferum which produce noscapine as the predominant alkaloid have also been grown commercially.

Modification of the morphine biosynthesis pathway in P. somniferum has primarily focused on conventional breeding approaches to increase the content of the desired alkaloid and reduce the content of unwanted contaminating alkaloids, identifying genes encoding enzymes active in the biosynthesis pathway for possible manipulation of them in order to accumulate the desired alkaloid at the expense of unwanted alkaloids, and the use of random mutagenesis to introduce mutations into the genome of P. somniferum followed by screening and selection of plants.

Growth regulators have also been used on P. somniferum to alter the alkaloid profile of plants and/or to increase the content of desired alkaloid(s) (see e.g., WO 2005/107436 and WO 2007/022561). In this instance, however, the change in alkaloid profile is not heritable requiring that plants be treated with the selected growth regulator in each growing season.

Whilst thebaine, for example, is not used therapeutically and has no analgesic or antitussive effect, it is an important precursor for the synthesis of 14-hydroxymorphinones such as oxycodone, naloxone, naltrexone, naltrexone methobromide, nalbuphine and nalmefene which have use as potent analgesics and/or narcotic antagonists. Other important opiate derivatives prepared from thebaine include buprenorphine and etophine. As another example, codeine is used to manufacture Active Pharmaceutical Ingredients (API) such as codeine phosphate, codeine sulfate, codeine hydrochloride and codeine base, which are in turn used in the manufacture of e.g., high-volume, over-the-counter (OTC) dosage forms for the relief of pain and cough (antitussives). Codeine is also the starting material and prototype of a large class of mainly mild to moderately strong opioids such as dihydrocodeine and hydrocodone and its derivatives such as nicocodeine and oxycodone.

The growing of P. somniferum remains the main method for the production of morphine and input opiates such as thebaine and codeine as synthetic methods whilst available, generally suffer from difficulty of synthesis, low yields, cost, utilise water-immiscible solvents creating safety concerns and environmental burdens, and/or result in the production of impurities and undesirable side products which can be difficult to remove leading to further losses in yield, and impose waste disposal and further environmental concerns.

Alkaloids are extracted from the poppy capsules of Papaver somniferum by two commercial methods. In one method, the immature capsule is cut, and the latex exudate from the wound is collected and dried to form opium. In the second method, the mature poppy capsules and the poppy capsule stems are collected, and threshed to remove the seeds and form a straw. When necessary, the straw is dried so as to have a water content below 16%. The alkaloids are then extracted from the straw or opium.

Where solvent or water or super critical fluid, such as CO₂, extraction is employed to remove the alkaloids from the straw, such method, as practiced, involves the production of “Concentrate of Poppy Straw”. Concentrate of Poppy Straw (or “CPS”) is described as “The material arising when poppy straw has entered into a process for the concentration of its alkaloids, when such material is made available in trade,” (Multilingual dictionary of narcotic drugs and psychotropic substances under international control, United Nations, New York, 1983). Not inconsistent with the foregoing description, Concentrate of Poppy Straw is described as “the crude extract of poppy straw in either liquid, solid or powder form which contains the phenanthrene alkaloids of the opium poppy, 45 U. S. Federal Register 77466, Nov. 24, 1980”. When in liquid form, the liquid is preferably concentrated before entering into commerce. The generally preferred Concentrate of Poppy Straw is the powder form which results from removing the solvent or water following extraction of the poppy straw. According to the United Nations publication ‘Narcotic Drugs: Estimated World Requirements for 2007; Statistics for 2005 (E/INCB/2006/2)’, “Concentrate of Poppy Straw is the dried residue obtained through the extraction of alkaloids from poppy straw. Further increasing the absolute content and/or proportion of a particular alkaloid or combination of alkaloids relative to impurity alkaloids present in the poppy straw or latex of plants of P. somniferum would markedly simplify extraction and purification increasing efficiency, quality and throughput, lower costs, and/or result in higher yields.

### SUMMARY OF THE INVENTION

The present invention stems at least in part from the observation that deficient magnesium chelatase activity influences the production of alkaloids in Papaver somniferum and that this can result in an increase in the content of one or more alkaloids and/or alter the alkaloid profile in a plant of this species. This is indicative that the efficiency with which particular alkaloid(s) is synthesised is increased and/or that plant regulatory mechanisms have been modified by the deficiency in magnesium chelatase activity.

In particular, reducing expression or activity of magnesium chelatase activity in P. somniferum as described herein may provide for an increase in absolute alkaloid content of one or more desired alkaloids in poppy straw and/or poppy capsule latex of the plant, and/or the alkaloid profile of the plant may be altered whereby the level of confounding alkaloid(s) (i.e., alkaloid(s) that are present in the poppy straw or latex but which need to be separated from the alkaloid(s) of interest) may be reduced. By reducing the level of level of confounding alkaloids (either by increasing the level of the alkaloid(s) of interest and/or reducing the level of the confounding alkaloid(s) relative to the desired alkaloid(s)), the efficiency of extraction of the desired alkaloids may also be increased.

Advantageously, the invention further provides for transfer of the characteristic of reduced magnesium chelatase activity from a first parent plant of P. somnferum having a particular chemotype by crossing the plant with another parent plant of P. somniferum having a different chemotype to provide new plants of P. somniferum with an altered alkaloid content and/or profile. Likewise, the characteristic of reduced magnesium chelatase activity can be transferred by employing a second parent which produces the same alkaloid as the predominant alkaloid in their poppy straw or latex as the first parent plant but which may contain the alkaloid in different absolute levels, or wherein the parent plants otherwise exhibit a different alkaloid profile to one another and/or have other characteristic difference(s) between them.

Thus, from the above, the present invention in one or more embodiments allows for the provision of new plants of P. somniferum for providing higher yields of a particular alkaloid or combinations of alkaloids, having an altered alkaloid profile, and/or improving extraction of alkaloid(s) from poppy straw, concentrate of poppy straw, latex and opium of the plants.

More particularly, in an aspect of the present invention there is provided a plant of Papaver somniferum modified to have reduced expression or activity of magnesium chelatase relative to a wild-type P. somniferum whereby the plant upon the harvesting of its poppy capsules yields poppy straw having an altered alkaloid profile compared to the wild-type plant.

The alkaloid profile of the plant may comprise or consist of the isoquinoline alkaloids of the plant or one or more subclasses thereof, e.g., the benzylisoquinoline alkaloids, morphinan alkaloids, phthalideisoquinoline alkaloids, benzo[c]phenanthridine alkaloids, bisbenzylisoquinoline alkaloids, alkaloids in the noscapine biosynthesis pathway, alkaloids in the papaverine biosynthesis pathway, alkaloids in the sanguinarine biosynthesis pathway, a particular combination of ones of the foregoing subclasses, and/or acombination of specific alkaloids of the foregoing.

In particularly preferred embodiments, the alkaloid profile may comprise or consist of morphinan alkaloids, alkaloids in the morphine biosynthesis pathway, alkaloids in the noscapine biosynthesis pathway, or a combination of specific alkaloids of the foregoing.

The reduced expression or activity of magnesium chelatase encompasses blocking, supressing, knockdown or silencing expression of one or more subunits of magnesium chelatase resulting in loss of functional magnesium chelatase or loss of activity of the enzyme in a plant embodied by the invention whereby the alkaloid profile of the poppy is thereby altered relative to the wild-type plant. In other embodiments, one or more mutations may be introduced into at least one gene encoding for a magnesium chelatase subunit whereby the gene is expressed but wherein the expressed subunit is either not functional or the function of the subunit is otherwise impaired resulting in loss of magnesium chelatase activity. Hence, it will be further understood from the above that the term “reduced expression” in the context of magnesium chelatase encompasses both reduced expression of magnesium chelatase as a whole and reduced expression of one or more subunits of the enzyme. Further, in embodiments in accordance with the invention the reduction in expression or activity of magnesium chelatase may be partial or complete.

In at least some embodiments, the reduced expression or activity of magnesium chelatase is associated with a single gene of the plant. That is, the expression of the gene may be reduced or the product encoded by the gene although expressed is defective in its function such that expression or activity of magnesium chelatase in the plant is impaired.

Typically, the gene encodes a magnesium chelatase subunit and expression of the gene is reduced in the plant, or the gene is expressed and encodes a mutant form of the magnesium chelatase subunit, whereby magnesium chelatase activity in the plant is thereby reduced.

In another aspect of the invention there is provided a method for providing a plant of P. somniferum having an altered alkaloid profile, comprising:

a) exposing at least one poppy seed of a Papaver somniferum parent plant to a mutagenizing agent;

b) growing the at least one poppy seed exposed to the mutagenizing agent to produce one or more further plants, optionally through one or more self-fertilised generations; and

c) providing a plant from the one or more plants which is identified to have reduced expression or activity of magnesium chelatase activity relative to the parent plant whereby upon the harvesting of poppy capsules of the identified plant that plant yields a poppy straw having an altered alkaloid profile compared to the P. somniferum parent plant.

In another aspect of the invention there is provided a method for identifying a plant of Papaver somniferum having an altered alkaloid profile, comprising screening the plant for reduced expression or activity of magnesium chelatase whereby, upon the harvesting of poppy capsules of the plant, the plant yields a poppy straw having the altered alkaloid profile.

Typically, the identification of a plant as having an altered alkaloid profile as described herein comprises screening the plant for reduced expression or activity of magnesium chelatase. In at least some embodiments, the screening comprises screening for reduced expression of one or more subunits of magnesium chelatase, or for defective magnesium chelatase in the plant. This can involve screening for expression of a defective magnesium chelatase subunit (e.g., a CHLI-A subunit) itself, or for a mutant gene encoding the subunit or for a mutation in the gene wherein the gene or the mutation is associated with reduced expression or activity of magnesium chelatase.

In another aspect of the invention there is provided a method for providing a Papaver somniferum with an altered alkaloid profile, comprising reducing expression or activity of magnesium chelatase in a plant of P. somniferum whereby upon the harvesting of its poppy capsules, the plant yields a poppy straw having an altered alkaloid profile.

Typically, at least one mutation is introduced into a gene encoding for a magnesium chelatase subunit whereby expression of the gene is reduced or a mutant form of the subunit is expressed, whereby the activity of magnesium chelatase in the plant is reduced.

In another aspect of the invention there is provided a method for increasing the content of an alkaloid in a plant of Papaver somniferum, the method comprising modifying the plant to reduce expression or activity of magnesium chelatase in the plant.

In a least some embodiments, the content of the alkaloid in poppy straw and/or latex of the plant is increased relative to one or more other alkaloids in the poppy straw or latex. Various ways for modifying the plant to have reduced expression or activity of magnesium chelatase are available, including such as by random mutagenesis of the plant, by targeted mutagenesis of the plant employing recombinant techniques, or via crossing the plant with another P. somniferum having the characteristic of reduced expression or activity of magnesium chelatase as described herein.

Hence, in another aspect of the invention there is provided a method for providing a plant of Papaver somniferum with an altered alkaloid profile, comprising:

(a) cross-pollinating a first parent plant of Papaver somniferum modified to have reduced expression or activity of magnesium chelatase in the plant with a second parent plant of P. somniferum having the same or a different chemotype compared to the first parent plant, to produce a first generation descendent P. somniferum plant; and

(b) self-pollinating the first generation plant to produce a second generation descendent P. somniferum plant wherein the second generation plant upon the harvesting of its poppy capsules yields poppy straw having an altered alkaloid profile in the straw compared to the second parent plant.

In preferred embodiments, the second parent plant typically has a different chemotype compared to the first parent plant.

In embodiments in which the first and second parent plants have the same chemotype, the second parent plant may, for instance, have one or more different traits or phenotypic characteristics (e.g. other than alkaloid profile) compared to the first parent plant wherein the second generation descendent plant exhibits the altered alkaloid profile and one or more of the different traits. Typically, further generations of descendent plants from the second generation plant will also be produced wherein the altered alkaloid profile is exhibited by the one or more further generations of descendent plants.

Further, the present invention expressly extends to descendants of plants embodied by the invention wherein the descendent plant exhibits the reduced expression or activity of magnesium chelatase, or is heterozygous for a modified gene associated with the reduced expression or activity of magnesium chelatase.

In another aspect there is provided a method for providing a poppy straw, comprising obtaining the poppy straw from poppy capsules harvested from a plant embodied by the invention or from a plant provided by a method embodied by the invention.

In another aspect there is provided a method for providing an opium, comprising collecting latex from immature poppy capsules of a plant embodied by the invention or from immature poppy capsules of a plant provided by a method embodied by the invention, and drying the latex to provide the opium.

In another aspect there is provided a poppy straw from poppy capsules harvested from a plant embodied by the invention or from a plant provided by a method embodied by the invention.

In another aspect there is provided a latex for the extraction of one or more alkaloids, the latex being a latex from immature poppy capsules of a plant embodied by the invention or from immature poppy capsules of a plant provided by a method embodied by the invention.

In another aspect there is provided an opium obtained by drying a latex from immature poppy capsules of a plant embodied by the invention or from immature poppy capsules of a plant provided by a method embodied by the invention.

In another aspect there is provided a concentrate of poppy straw being a concentrate of the poppy straw of a plant embodied by the invention.

In another aspect there is provided an alkaloid extracted from a poppy straw, latex, opium, or concentrate of poppy straw embodied by the invention.

In another aspect there is provided seed from a plant embodied by the invention or from a plant provided by a method embodied by the invention.

In another aspect there is provided a plant cell or plant root from a plant embodied by the invention or from a plant provided by a method embodied by the invention.

There is also provided herein the use of a modified magnesium chelatase of a plant of P. somniferum in the production of one or more alkaloids of P. somniferum, wherein activity of the modified magnesium chelatase is reduced compared to the wild-type form of the enzyme.

In another aspect, there is provided a preparation of P. somniferum cells in the production of one or more alkaloids of P. somniferum, the cells comprising a modified magnesium chelatase wherein the activity of the magnesium chelatase is reduced compared to the wild-type form of the enzyme.

In another aspect, there is provided a composition comprising P. somniferum cells modified so as to exhibit reduced expression or activity of magnesium chelatase.

Typically, in embodiments as described herein, where the reduced expression or activity of magnesium chelatase is associated with a single gene of the plant, the gene is a recessive gene which encodes a mutant form of the magnesium chelatase subunit.

Typically, the subunit is a magnesium chelatase I-A (CHLI-A) subunit.

In at least some embodiments, the CHLI-A subunit is truncated compared to the wild-type form of the subunit e.g., by the introduction of a stop codon into the gene.

In at least some embodiments, the CHLI-A subunit has a nucleic acid sequence as set forth in SEQ ID NO: 2.

Typically, the wild-type form of the CHLI-A subunit has an amino acid sequence as set forth in SEQ ID NO: 3.

Typically, the plant has a further gene encoding a magnesium chelatase I-B (CHLI-B) subunit which whilst expressed in the plant, magnesium chelatase activity in the plant remains deficient.

Typically, the CHLI-B subunit has greater than 99% amino acid sequence identity with the amino acid sequence of the CHLI-A subunit as set forth in SEQ ID NO: 3.

Typically, the CHLI-B subunit has an amino acid sequence as set forth in SEQ ID NO: 8 and 100% amino acid sequence identity with the wild-type form of the CHLI-B subunit.

Typically, a plant embodied by the invention will have an altered morphinan alkaloid profile compared to the wild-type parent plant.

In particularly preferred embodiments, a plant of P. somniferum in accordance with the invention is a field-grown plant.

In at least some embodiments in accordance with the invention, the plant may have a chemotype in which the predominant alkaloid in poppy straw or latex of the plant is selected from, but not limited to, thebaine, morphine, oripavine, codiene and noscapine. Typically, a plant embodied by the invention has a thebaine or morphine chemotype. Most typically, the plant has a thebaine chemotype.

Similarly, the parent plant embodied by the invention used for crossing with a wild-type P. somniferum as described herein will generally have a thebaine or codeine chemotype although the use of a parent plant of the invention having a different chemotype e.g., one in which morphine or noscapine is the predominant alkaloid, is also expressly encompassed.

In other embodiments, a plant in accordance with the invention may have a codeine chemotype. In such embodiments, the plant may be a provided by transfer of the reduced expression or activity of magnesium chelatase characteristic into the plant from a P. somniferum with a non-codeine phenotype (e.g., from a plant with a thebaine, morphine or noscapine chemotype). A plant of P. somniferum with a codeine chemotype and having reduced expression or activity of magnesium chelatase as described herein may also be crossed with a second parent P. somniferum having a different chemo type (e.g., a thebaine chemotype) in accordance with a method embodied by the invention.

Still further, a plant embodied by the invention or provided by a method in accordance with the invention can have an alkaloid profile in poppy straw or latex of the plant which comprises one or more benzylisoquinoline and/or phthalideisoquinoline alkaloids selected from, but not limited to morphine, oripavine, codeine, thebaine, reticuline, papaverine, salutaridine, laudanine, sanguinarine and noscapine, wherein the alkaloid profile differs in absolute content or proportion of one or more of the alkaloids compared to the wild-type plant.

Typically, a plant embodied by the invention has an alkaloid profile in which the absolute content or proportion of alkaloids in an alkaloid combination comprising morphine, oripavine, codeine and thebaine (MOCT) in the poppy straw or latex of the plant is altered compared to the wild-type plant. In other embodiments, the alkaloid combination may further comprise one or more of noscapine, papaverine, reticuline, papaverine, salutaridine, laudanine and sanguinarine.

Typically, the reduction or activity of magnesium chelatase activity in a plant as described herein manifests in light leaf colour phenotype of the plant compared to the wild-type P. somniferum, and it was this lighter leaf colour that was the initial feature which drew the attention of the inventors and led to the present invention. The lighter leaf colour becomes particularly apparent when the plants are grown in the field rather than a plant house (e.g., a plant glass house or polyhouse).

Despite the substantially lighter leaf colour, plants as described herein can nevertheless still exhibit good vigour and so be suitable for use as a field crop. This is surprising as such plants would normally be expected to have low vigour as light leaf colour is suggestive of a serious defect such as low leaf chlorophyll or a severe nutrient deficiency, which would be expected to be reflected in poor growth rates with consequential low capsule number, poor straw and alkaloid content and/or yield. Moreover, generally when seeking to develop Papaver somniferum for the production of alkaloid(s) such plants would be discarded at an early stage of the program once the lightness of the leaf colour had become apparent. However, in contradiction to this, in arriving at the present invention, the inventors cultivated the plants to maturity and further investigated their alkaloid profile leading to the unexpected finding that the plants can nevertheless exhibit excellent alkaloid content despite the light leaf colour of the plants. Thus, in at least some embodiments of the invention, light leaf colour as a result of reduced expression or activity of magnesium chelatase may act as a biomarker for increased alkaloid content and/or altered alkaloid profile in poppy straw and/or poppy capsule latex.

Thus, in another aspect of the invention there is provided herein a plant of Papaver somniferum having an introduced trait for a lightened leaf colour associated with reduced expression or activity of magnesium chelatase in the plant.

In another aspect there is provided herein a plant of Papaver somniferum having an introduced trait for a lightened leaf colour associated with reduced expression or activity of magnesium chelatase in the plant whereby the plant upon the harvesting of its poppy capsules will yield poppy straw having an altered alkaloid profile.

In another aspect there is provided herein a plant of Papaver somniferum having an introduced trait for a lightened leaf colour associated with expression of a magnesium chelatase having deficient activity in the plant whereby the plant upon the harvesting of its poppy capsules yields poppy straw having an altered alkaloid profile.

In another aspect there is provided herein a plant of Papaver somniferum wherein the plant is modified in a gene encoding a magnesium chelatase subunit wherein expression or activity of magnesium chelatase in the plant is reduced compared to wild-type magnesium chelatase expression or activity and the plant upon the harvesting of its poppy capsules will thereby yield a poppy straw having an altered alkaloid profile.

In another aspect there is provided herein a plant of Papaver somniferum wherein the plant expresses a defective magnesium chelatase subunit compared to the wild-type form of the subunit wherein the activity of magnesium chelatase in the plant is reduced and the plant upon the harvesting of its poppy capsules will thereby yield a poppy straw having an altered alkaloid profile.

The invention in particular extends to plants of Papaver somniferum that are stably reproducible in respect of the altered alkaloid profile of the plants embodied by the invention. Likewise, the invention expressly extends to plants of P. somniferum in which the introduced trait or modification associated with the altered alkaloid profile is stably heritable.

One of ordinary skill in the art will understand that the total alkaloid content in the poppy straw, concentrate of poppy straw, latex or opium in embodiments of the present invention will total (but will not exceed) 100%.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers, integers or steps.

Any discussion of documents, acts, materials, devices, articles or the like that has been included in this specification is solely for the purpose of providing a context for the invention. It is not to be taken as an admission that any or all of these matters form part of the prior art base or were common general knowledge in the field relevant to the invention as it existed in Australia or elsewhere before the priority date of this application.

The features and advantages of the invention will become further apparent from the following detailed description of embodiments thereof together with the accompanying drawings.

### BRIEF DESCRIPTION OF THE ACCOMPANYING DRAWINGS

FIG. 1 is a graph showing codeine and thebaine content in the mature capsules of 20 M3 Papaver somniferum mutant plant lines in a field trial (Hagley, Tasmania, Australia) conducted during the 2012/13 poppy growing season. The lines PW08-2308, PW10-0149 and PW11-4063 are ‘Tasman’ Papaver somniferum exhibiting a codeine chemotype.

FIG. 2 is a photograph showing ‘Tasman’ M3 mutant line EM4-0045 (middle foreground) during a disease resistance screening field trial (Hagley, Tasmania, Australia) conducted during the 2012/13 poppy growing season. The EM4-0045 line exhibited pale, light-green leaves compared to the parent ‘Tasman’ line (PW08-2308) from which it was derived and other Papaver somniferum lines in surrounding plots not expressing the lighter leaf colour trait of the EM4-0045 line, and was found to have exceptionally high codeine and low thebaine content in its mature capsules.

FIG. 3 is a further photograph clearly showing the substantially lighter leaf colour of field grown EM4-0045 plants (left plot) compared to the typical darker leaf colour phenotype of commercially grown ‘Tasman’ P. somniferum. In this example, the ‘Tasman’ line EM3-1217 (right plot) shown. Both lines were derived from the same ‘Tasman’ parent line, PW08-2308.

FIG. 4 is a three-dimensional plot of L*a*b* values for each of ten PW08-2308, EM4-0045 and EM3-0006 line leaf samples, as well as for each of nine ‘Ted’ P. somniferum PW07-0355 and ten EM4-019 line leaf samples. All leaves are from planthouse grown plants.

FIG. 5 is a photograph showing two leaves each of EM4-0045 (left) and EM3-006 (right) planthouse grown plants.

FIG. 6A shows a protein sequence alignment of magnesium chelatase subunit I genes. PsCHLI-A (SEQ ID No: 3) and PsCHLI-B (SEQ ID No: 8) are P. somniferum genes (wild-type PH11-0943 line protein sequences), TAIR accession AT4G18480.1 is Arabidopsis thaliana CHlI-1(SEQ ID No: 27) (The Arabidopsis Information Resource (TAIR), Phoenix Bioinformatics, Fremont, Calif., USA; www.arabidopsis.org), TAIR accession AT5G45930.1 is A. thaliana ChlI-2 (SEQ ID No: 28), NCBI accession AET86637 is Pisum sativum (pea) ChlI (SEQ ID No: 29) (National Center for Biotechnology Information (NCBI), 8600 Rockville Pike, Bethesda, Md., USA) and NCBI accession NP_001347251 (SEQ ID No: 30) is Glycine max (soybean) ChlI. The position of the Q328* mutation detected in the light green-yellow line EM4-0045 is indicated by the box-shaped annotation at position 333 in the alignment. The amino acid sequences for each of (a) AT4G18480.1 (SEQ ID No: 27), (b) AT4G18480.1 (SEQ ID No: 28, (c) AET86637 (SEQ ID No: 29) and (d) NP_001347251 (SEQ ID No: 30) are shown in FIG. 6B.

FIG. 7 shows three leaves each of ‘Ted’ P. somniferum PW07-0355 (left) 0355 and EM4-0019 lines (right). The leaves of the EM4-0019 line are paler than the leaves of the PW07-0355 parent line.

FIG. 8 shows the gDNA sequence of wild-type TAgene201937 (PsCHLI-A) (SEQ ID NO: 1; 1,750bp). Coding regions are shaded and underlined. The position of an EMS induced ‘C to T’ mutation in EM4-0045 (position 1,319 in SEQ ID NO: 1) is indicated in enlarged font within the sequence.

FIG. 9 shows the cDNA sequence of wild-type TAgene201937 (PsCHLI-A) (SEQ ID NO: 2; 1,278bp). The position of the EMS ‘C to T’ mutation in EM4-0045 is indicated in enlarged font within the sequence.

FIG. 10 shows the predicted protein sequence of wild-type TAgene201937 (PsCHLI-A) (SEQ ID NO: 3; 425aa). The position of the EM4-0045 mutation (Q328*) is indicated in enlarged font within the sequence.

FIG. 11 shows the gDNA sequence of EM4-0045 TAgene201937 (PsCHLI-A) (SEQ ID NO: 4; 1;715bp). Bases in enlarged font represent SNPs relative to wild-type PsCHLI-A (PH11-0943)

FIG. 12 shows the gDNA sequence for the TAgene201937 (PsCHLI-A) (SEQ ID NO: 5; 1,530bp) of the ‘Ted’ Papaver somniferum line PW13-4611. Bases in enlarged font represent SNPs relative to wild-type PsCHLI-A (PH11-0943).

FIG. 13 shows the gDNA sequence of wild-type TAgene224147 (PsCHLI-B) (SEQ ID NO: 6; 1,641bp). Coding regions are shaded and underlined.

FIG. 14 shows the cDNA sequence of wild-type TAgene224147 (PsCHLI-B) (SEQ ID NO: 7; 1,278bp).

FIG. 15 shows the predicted protein sequence of wild-type TAgene224147 (PsCHLI-B) (SEQ ID NO: 8; 425aa).

FIG. 16 shows the coding gDNA region of TAgene224147 (PsCHLI-B) EM4-0045 allele 2 (SEQ ID NO: 9). A single SNP relative to the wild-type sequence is indicated in enlarged font within the sequence.

FIG. 17 shows the predicted protein sequence of TAgene224147 (PsCHLI-B) EM4-0045 allele 2 (SEQ ID NO: 10). A single amino acid change relative to the wild-type protein sequence is indicated in enlarged font within the sequence (D110G).

FIG. 18 shows the coding gDNA region of TAgene224147 (PsCHLI-B) EM4-0045 allele 3 (SEQ ID NO: 11). Three SNPs relative to the wild-type sequence are indicated in enlarged font within the sequence.

FIG. 19 shows the predicted protein sequence of TAgene224147 (PsCHLI-B) EM4-0045 allele 3 (SEQ ID NO: 12). Three amino acid changes relative to the wild-type protein sequence are indicated in enlarged font within the sequence (V236G, D237E and V238G).

### DETAILED DESCRIPTION OF EXEMPLARY EMBODIMENTS OF THE INVENTION

Chlorophylls are tetrapyrrole structured compounds and one of two major tetrapyrrole-based classes of compounds produced by photosynthetic organisms. Both tetrapyrrole biosynthetic pathways (chlorophyll and heme) share several common intermediates before branching at protoporphyrin IX (Proto). The insertion of magnesium (Mg²⁺) into protoporphyrin IX by magnesium chelatase is the first committed step of chlorophyll biosynthesis. Following this, chlorophyll is synthesized from the subsequent modification of Mg-protoporphyrin IX and the further downstream processing of intermediates by reduction and esterification (Walker CJ and Willows RD (1997) Mechanism and regulation of Mg-chelatase. Biochem. J., 327, 321-333).

Magnesium chelatase is a multicomponent enzyme consisting of at least three separable proteins, or subunits, known as CHLI, CHLD and CHLH (Walker and Willows 1997, vide supra). In the vascular plant Arabidopsis thaliana and in the green alga Chlamydomonas reinhardtii, one gene encoding for the CHLD subunit has been identified whilst two genes each for the CHLH and CHLI subunits have been found to exist (Brzezowski P, Sharifi MN, Dent RM, Morhard MK, Niyogi KK and Grimm B (2016) Mg chelatase in chlorophyll synthesis and retrograde signaling in Chlamydomonas reinhardtii: CHLI2 cannot substitute for CHLI. Journal of Experimental Botany, 67, 3925-3938).

Disrupted magnesium chelatase activity owing to mutations within magnesium chelatase subunit genes have been reported to result in chlorophyll deficient phenotypes, and chlorophyll deficient mutants having either recessive or semi-dominant modes of inheritance have been described in a number of plants species, including Arabidopsis, rice, barley, maize, tobacco and soybean (see Campbell BW, Mani D, Curtin SJ, Slattery RA, Michno J-M, Ort DR, Schaus PJ, Palmer RG, Orf JH and Stupar RM (2015) Identical substitutions in magnesium chelatase paralogs result in chrolophyll-deficient soybean mutants. G3, 5, 123-131. doi: 10.1534/g3.114.015255). In all cases, the magnesium chelatase mutants have a yellow, or much lighter green, color phenotype in comparison to typical wild-type plants of the species.

P. somniferum plants having either light yellow (Singh HP, Tiwari RK, Singh SP and Singh AK (2002) Inheritance of light yellow leaf colour in opium poppy (Papaver somniferum L.) Indian J. Genet. 62(2), 181-182) or yellowish green leaves (Dubey MK, Shasany AK, Dhawan OP, Shukla AK, Khanuja SPS (2009) Genetic variation revealed in the chloroplast-encoded RNA polymerase β′ subunit of downy mildew-resistant genotype of opium poppy. Journal of Heredity, 100(1), 76-85) have also been reported. In both of these cases the P. somniferum plants were reported to respectively contain approximately half the chlorophyll content of normal wild-type varieties though the basis for the reduction in chlorophyll was not identified nor do these reports provide any suggestion or consideration of any possible improved alkaloid profile outcomes as a consequence or reduced chlorophyll content in poppy.

The magnesium chelatase step in chlorophyll biosynthesis is reported to be tightly regulated with both the products and substrates of the reaction suggested to regulate early steps in the tetrapyrrole biosynthetic pathway and be involved in nuclear genome signaling (Gao M, Hu L, Li Y and Weng Y (2016) The chlorophyll-deficient golden leaf mutation in cucumber is due to a single nucleotide substitution in CsChlI for magnesium chelatase I subunit. TAG, 129, 1961-1973. DOI 10.1107/s00122-016-2752-9). Signaling from the chloroplast organelle to the cell nucleus is termed retrograde signaling and functions to communicate and coordinate nuclear-encoded adaptive responses to, for example, perturbations in chloroplast homeostasis (Chan KX, Phua SY, Crisp P, McQuinn R and Pogson BJ (2016) Learning the languages of the chloroplast: Retrograde signaling and beyond. Annual Review of Plant Biology, 67, 25-53; de Souza A, Wang J-W and Dehesh K (2017) Retrograde signals: integrators of interorganellar communication and orchestrators of plant development. Annual Review of Plant Biology, 68, 85-108).

One such adaptive response is to regulate the expression of genes associated with photosynthetic protein complexes and function, so-called photosynthesis-associated nuclear genes or PhANGs. A recent study in pea (Pisum sativum) exemplifies this response, with many PhANG genes being downregulated in yellow-leaf pea plants which had been silenced for CHLD activity through virus-induced gene silencing (VIGS) (Luo T, Luo S, Araujo WL, Schlicke H, Rothbart M, Yu J, Fan T, Fernie AR, Grimm B and Luo M (2013) Virus-induced gene silencing of pea CHLI and CHLD affects tetrapyrrole biosynthesis, chloroplast development and the primary metabolic network. Plant Physiology and Biochemistry, 65, 17-26). Increased levels of 2-oxogluturate (6.3X) and shikimate pathway amino acids (e.g. tryptophan 3.7X and phenylalanine 2.4X) in yellow-leafed CHLI-silenced pea plants were also reported in this study. Benzylisoquinoline biosynthesis begins with the condensation of two L-tyrosine derivatives (an amino acid of the shikimate branch) and 2-oxogluturate serves as an essential co-factor to two key 2-oxogluturate/Fe(II)-dependent dioxygenase enzymes functioning within the morphinan BIA pathway (i.e., thebaine 6-O-demthylase and codeine O-demethylase; Beaudoin GA and Facchini PJ (2014) Benzylisoquinoline alkaloid biosynthesis in opium poppy. Planta, 240(1), 19-32).

Jasmonic acid is additionally produced within the chloroplast. This plant hormone has been implicated in retrograde signaling (de Souza et al. 2017, vide supra), and may have involvement in the transcriptional regulation of benzylisoquinoline alkaloid (BIA) synthesis (Yamada Y and Sato F (2013) Transcription factors in alkaloid biosynthesis. International Review of Cell and Molecular Biology, 305, 339-382).

Without being limited by theory, it is thought that the altered alkaloid profiles in P. somniferum plants embodied by the present invention stem from deficient chloroplast processes of the plants resulting from the introduced disruption in magnesium chelatase activity, which may affect alkaloid synthesis by virtue of retrograde signaling mechanisms that influence substrate/co-factor availability and/or regulate expression of genes encoding for enzymes involved in the synthesis of alkaloids, and particularly BIA gene expression.

Codeine, for example, is an intermediate in the morphine biosynthesis pathway in Papaver somniferum. The morphine biosynthesis pathway is highly complex involving multiple alkaloid intermediates and enzymatic steps, and is but one of a number of pathways present in Papaver somniferum involving the synthesis of benzylisoquinoline alkaloids. In the morphine biosynthesis pathway (S)-reticuline is converted to (R)-reticuline via 1,2-dehydroreticuline. (R)-reticuline leads to the synthesis of the morphine via salutaridine, salutaridinol, salutaridinol-7-O-acetate, and thebaine which is a branch point with one branch leading to morphine via oripavine and morphinone and the other branch leading to morphine via neopinone, codeinone and codeine (see Scheme 1 below). Morphine and the alkaloids leading to its synthesis from (R)-reticuline comprise the morphinan subclass of benzylisoquinoline alkaloids.

The various biosynthesis pathways within P. somniferum are interlinked and alkaloid in one pathway can also be intermediates in another pathway. For example, (S)-reticuline is a branch point between the benzylisoquinoline and phthalideisoquinoline alkaloid pathways and is converted to (S)-scoulerine by the berberine bridge enzyme (BBE), which leads to the production of noscapine via the intermediates (S)-tetrahydrocolumbamine and (S)-canadine. The benzylisoinoline alkaloid laudanine is also derived from (S)-reticuline, whilst (S)-scoulerine is an intermediate in the production of sanguinarine via a number of alkaloids such as protopine. See e.g., Ziegler J and Facchini PJ., (2008) “Alkaloid Biosynthesis: Metabolism and Trafficking”, Annu. Rev. Plant Biol. 59:735-769.

Whilst substantial progress has been made in elucidating the steps and identifying the intermediate alkaloids involved in these pathways, their regulation remains poorly understood as a result of the interplay of branching of the pathways combined with the involvement of as yet unraveled feedback and homeostatic mechanisms. This is further complicated by factors such as the potential for an enzyme to have a role in the synthesis of more than one alkaloid intermediate and, for example, the tissue-specific synthesis of alkaloids. Consequently, the effect of changes introduced into alkaloid synthesis pathways of plants of Papaver somniferum by reducing expression or activity of magnesium chelatase activity as described herein may result in noticeable changes in the alkaloid profiles of the plants as described herein. For instance, the absolute content of one or more alkaloids in latex from immature poppy capsules or in poppy straw may be increased and/or the proportion of one or more alkaloids relative to one or more other alkaloids may be changed compared to the wild-type plant.

In general usage the term “wild-type” refers to the phenotype of the typical form of the species as it occurs in nature, and for a P. somniferum this refers to a plant which accumulates morphine as its predominant alkaloid within the matured capsule(s) (i.e., a morphine chemotype). As used herein, however, the term “wild-type plant” is to be taken to refer to the parent plant (e.g., from a P. somniferum plant line) irrespective of the parent plant's chemotype (e.g., morphine free varieties), from which the plant of the invention is derived and to which the reduced expression or activity of magnesium chelatase activity and/or altered alkaloid profile of the plant of the invention is compared.

Persons skilled in the art will be able to readily grow plants of Papaver somniferum embodied by the invention and reproduce them, collect latex or produce poppy straw from them, and purify alkaloid(s) from the latex (or opium) or the straw in accordance with the invention.

Seed of a plant of Papaver somniferum (EM4-0045) which is homozygous for a defective psCHLI-A subunit found to be associated with reduced magnesium chelatase activity as described herein has been deposited under the Budapest Treaty with the National Collection of Industrial, Food and Marine Bacteria (NCIMB Ltd, Ferguson Building, Craibstone Estate, Bucksburn, Aberdeeen AB21 9YA, Scotland, United Kingdom), on 24 Aug. 2016, under Accession No. NCIMB 42630. The availability of these seeds is not to be construed as a license to practice the present invention in contravention of rights granted under the authority of any government in accordance with its patent or breeder's rights laws.

As one enablement of the invention, and as further described below, plants grown from the seed of Papaver somniferum line EM4-0045 may be crossed with plants of a wild-type second parent P. somniferum having a different chemotype (e.g., plants in which the predominant alkaloid produced is thebaine) to produce progeny plants which are grown and self-pollinated to produce a further generation of plants having the chemotype of the second parent wild-type plant but which has reduced magnesium chelatase activity and an altered alkaloid profile compared to the second parent plant.

In a particularly preferred embodiment, a plant embodied by the invention can be provided by subjecting seed a Papaver somniferum parent plant as described herein to mutagenesis. The production of mutagenized seed is well known in the art. Methods of seed mutagenesis as well as mutagens suitable for use in these methods, such as, ethyl methanesulfonate (EMS), are described in the Manual on Mutation Breeding, 2nd ed., I.A.E.A., Vienna 1977 or in Plant Breeding, Principles and Prospects, Chapman and Hall, London 1993. For mutagenesis of seed by X-ray, hydrated seeds may be treated with 20,000 rads, (30 cm from the source for 45 minutes using a filter). X-ray mutagenesis is described and compared to EMS mutagenesis by Filippetti, A. et al., “Improvement of Seed Yield in Vicia Faba L. By Using Experimental Mutagenesis II Comparison of Gamma-Radiation and Ethyl-Methane-Sulphonate (EMS) in Production of Morphological Mutants”, Euphytica 35 (1986) 49-59. DEB (diepoxybutane) mutagenized seeds may, for example, be obtained by soaking the seeds in water overnight, then soaking in 22 mM DEB for 4 hours, followed by extensive washing. Other mutagens which may be utilized include ethyl-2-chloroethyl sulphide, 2-chloroethyl-dimethylamine, ethylene oxide, ethyleneimine, dimethyl sulphonate, diethyl sulphonate, propane sulphone, beta-propiolactone, diazomethane, N-methyl-N-nitrosourethane, acridine orange and sodium azide.

Mutagenesis utilizing EMS is well described in the literature. The Manual on Mutation Breeding, supra, reports a preferred EMS mutagenesis process for barley seeds as practiced by K. Mikaelson. In this preferred process, the seeds are prepared, pre-soaked, treated with the mutagen and post-washed.

U.S. Pat. No. 6,067,749, incorporated by reference herein in its entirety, describes the use of EMS for the preparation of a Papaver somniferum strain with a high concentration of thebaine and oripavine.

Irradiation methods such as fast neutron mutagenesis (“FNM”) may also be used to produce mutagenized seed (see e.g., Li, X. et al., A fast neutron deletion mutagenesis-based reverse genetics system for plants, The Plant Journal 27(3), 235-242 (2001)). Fast neutron mutagenesis is, for instance, described by Kodym and Afza (2003), Physical and Chemical Mutagenesis, pp 189-203, in Methods in Molecular Biology, Vol. 236: Plant Functional Genomics: Methods and Protocols (Ed. E. Grotewold), Humana Press Inc, Totowa, N.J.

Gamma (γ) rays are electromagnetic waves of very short wavelengths and are obtained by disintegration of radioisotopes Co or Cs. γ sources can be installed in a γ cell, a γ room, or γ field. These are shielded by lead or concrete. Most γ sources are suitable for seed irradiation, as long as the size of irradiation space is sufficient and the dose rate allows practical irradiation times. In contrast, fast neutrons are uncharged particles of high kinetic energy and are generated in nuclear reactors or in accelerators. The skilled person should assess the feasibility for seed irradiation with the operators, since not all facilities are suitably equipped and can produce fast neutrons at a low degree of contamination with other radiation.

The two radiation types differ in their physical properties and hence, in their mutagenic activity. γ rays have a lower relative biological effectiveness (RBE) than fast neutrons, which implies that in order to obtain the same biological effect, a higher dose of γ radiation must be given. RBE is mainly a function of the linear energy transfer (LET), which is the transfer of energy along the ionizing track. γ rays produce a few ionizations per micron of path (low LET) and belong to the category of sparsely ionizing radiation. Fast neutrons (high LET, densely ionizing radiation) impart some of their high kinetic energy via collisions, largely with protons within the material. When radiation passes through tissue, physical events such as ionizations (ejection of electrons from molecules) and excitations (process of raising electrons to a higher energy state) occur and lead to effects in DNA, membranes, lipids, enzymes, etc. Secondly, chemical events are induced that start with the formation of activated molecules, so-called free radicals (OH. and H.) that arise from OH− and H+. If oxygen is present, it reacts readily with radiation-induced free radicals to form peroxyradicals. In the case of low LET radiation, the formation of peroxyradicals is favoured. In high LET radiation, the formation of hydrogen peroxide (H₂O₂) by recombination of free radicals is favoured. All radicals and hydrogen peroxide can react with biological molecules. Primary damage caused by radiation occurs randomly and is both physiological and genetic. Physiological recovery and repair of DNA are possible to some extent, as non-damaged molecules may take over metabolic processes and DNA repair mechanisms are activated.

Before starting any mutation induction studies, it is most crucial to select suitable doses. For mutation induction, it is advisable to use two to three doses along with a control. The applicable doses will depend on the breeding or research objective, the radiation type and the particular plant material. It is known that plant genera and species and, to a lesser extent, cultivars differ in their radiosensitivity. Radiosensitivity (radiation sensitivity) is a relative measure that gives an indication of the quantity of recognizable effects of the radiation exposure on the irradiated object. The radiosensitivity is influenced by biological factors (such as genetic differences, nuclear and interphase chromosome vol) and by environmental modifying factors (oxygen, water content, post-irradiation storage, and temperature).

Modifying factors greatly affect mutagenic efficiency and reproducibility of results. Oxygen is the major modifying factor, while moisture content, temperature, and storage appear to be secondary, interacting with the oxygen effect. Oxygen shows synergistic action with sparsely ionizing radiation, but oxygen effects during irradiation and post-irradiation storage can easily be prevented by adjustment of seed water content to 12-14% in cereals and most other seeds. In oilseeds such as poppies, the seed water content should be lower, around 7-8%. The critical region is the embryo, but it can be assumed that the water content of the seed and the embryo of most species will be similar. Environmental factors are less important with densely ionizing radiation; thus, for fast neutron radiation, no seed moisture adjustment is necessary.

Unless data on the radiosensitivity of a given plant are already published or known from experience, the mutation induction program should be preceded by a radiosensitivity test. This is done by irradiating the seeds with a range of doses and by growing out the plants under greenhouse conditions. Radiosensitivity is assessed based on criteria such as reduced seedling height, fertility, and survival in the M1 generation. A seedling height reduction of 30-40% is generally assumed to give a high mutation yield. The usefulness of radiation can be judged by mutagenic efficiency, which is the production of desirable changes free from association with undesirable changes. A high dose will increase mutation frequency (the frequency at which a specific kind of mutation or mutant is found in a population of cells or individuals), but will be accompanied by negative features, such as sterility. When selecting the doses, it will be necessary to find a treatment regime providing high mutagenic efficiency.

For fast neutron radiation, dosimetric measurements have to be done during each radiation treatment, e.g., by performing the sulphur threshold detector method, since the neutron flux in the seed irradiation unit is not constant.

The Gray (symbol Gy), the SI (Systéme Internationale) unit used to quantify the absorbed dose of radiation (1 Gy=1 J/kg) replaced the old unit rad; 1 Gy=100 rads or 1 krad=10 Gy. The absorbed dose rate (Gy/s or Gy/min) indicates how much energy the irradiated material absorbs during a given unit of time. The length of exposure and the dose rate determines the radiation dose. Exposure during short times (seconds to a few hours) at a high dose rate is referred to as acute and is most applied in irradiation programs.

Fast neutrons have been shown to be a very effective mutagen. Koornneef et al. (1982) found that about 2500 lines treated with fast neutron at a dose of 60 Gy are required to inactivate a gene once on average (Koornneef, M., Dellaert, L. W. M. and van der Veen, J. H. (1982) EMS- and radiation-induced mutation frequencies at individual loci in Arabidopsis thaliana (L.) Heynh. Mutat. Res. 93, 109-123). If the plant genome contains about 25000 genes, it is estimated that about 10 genes are randomly deleted in each line.

FNM offers a number of advantages over using chemical treatment such as EMS. Notably, the treatment is applied to the dried seed, which can be sown at a later date, while with EMS the seed needs either to be sown immediately after treatment, or carefully re-dried for sowing later. However, chemical mutagenesis is particularly useful and methods embodied by the invention are exemplified herein by treatment of seeds with EMS.

After exposing seeds to a mutagen in accordance with a method embodied by the invention, the seeds are typically grown to maturity in controlled conditions and then self-pollinated. The seeds from the mature plant are taken and at least one seed is planted to grow an M2 generation. The M2 generation is screened for alkaloid production. Of course, it is possible to screen the M1 generation, but there are several advantages to screening the M2 generation. Firstly, screening the M2 generation insures that the trait resulting from mutagenesis can be inherited. Secondly, by growing the M2 generation, the basic hardiness of the plant is proven before screening. Thirdly, traits resulting from mutagenesis are generally inherited as recessive genes. Typically, the mutated gene will be in the heterozygous state in the M1 generation, and thus the mutation will be masked by the dominant (non-mutated) form of the gene. In the M2 generation, however, in a proportion of the plants the gene will be in the homozygous state, and the effect of the mutation apparent.

The M2 plants can be grown to produce an immature capsule, but it is possible to save time and labor if the plants are screened at an earlier stage of growth.

It is recommended that the plants be screened at a point beginning at the 6 leaf stage, up to the 10 leaf stage. Screening at this early stage advantageously allows many plants to be managed in a small space.

Plants embodied by the invention may also be provided by modifying a plant of P. somniferum to have reduced expression of magnesium chelatase activity as described herein via other methods known to the persons in this field of this invention, such as by directed mutation of any of the magnesium chelatase subunit genes through use of gene editing methods including zinc finger nucleases (ZFNs), transcription activator-like effector nucleases (TALENs), and clustered regularly interspaced short palindromic repeats/Cas9 (CRISPR/Cas9) (Kamburova VS et al. (2017) Genome Editing in Plants: An Overview of Tools and Applications. International Journal of Agronomy, https://doi.org/10.1155/2017/7315351). It will also be recognized that by combining methods of conventional mutagenesis which introduce random mutations (e.g., EMS) with next-generation sequencing (NGS) technologies to screen gene regions of interest, it is possible to rapidly screen large numbers of mutated individuals for mutations within gene(s) of interest (Sikora P et al (2011) Mutagenesis as a Tool in Plant Genetics, Functional Genomics, and Breeding. International Journal of Plant Genomics, doi:10.1155/2011/314829), and that such strategies could be employed to identify P. somniferum plants having mutations within the magnesium chelatase gene sequences described herein. Further, RNA-induced gene silencing (RNAi) in which RNA molecules inhibit gene expression or translation by neutralizing targeted messenger RNA (mRNA) molecules has been shown to be effective in silencing one or multiple genes in plants (McGinnis KM (2010) RNAi for functional genomics in plants. Briefings in Functional Genomics, 9(2), 111-117; doi:10.1093/bfgp/elp052), and so represents another method available to the skilled addressee for reducing magnesium chelatase activity in P. somniferum as described herein.

The screening of plants is generally labor intensive and as such, to improve return on labor, generally only plants that appear healthy have conventionally been screened. However, the present inventors in arriving at the present invention and contrary to conventional practice, screened plants as part of a mutagenesis study which had substantially lighter colour leaves than the parent plant and other plant lines generated as described above. As the lighter colour could indicate a serious defect such as a nutrient deficiency or a reduction in chlorophyll content in their leaves, these plants would have been expected to have reduced vigour, which would adversely impact on commercially important characteristics of the plants such as one or more of rate of maturation, capsule number, poppy straw and/or latex yield and thereby, overall codeine yield. Nevertheless, and contrary to expectations, the present inventors found that such plants could have an altered alkaloid profile in which the absolute content of alkaloid of interest was increased compared to the wild-type parent plant, and so be useful as a commercial field crop.

In at least some embodiments, in the screening process, each plant is measured for content of the alkaloid or combination of alkaloids of interest optionally relative to one or more confounding alkaloids. For example, the content of thebaine can be measured relative to morphine, codeine and/or oripavine content. Additional confounding alkaloids such as e.g., papaverine and noscapine can also be measured.

This can be accomplished by extracting, for example, poppy straw into a liquid buffer or by dissolving a latex sample into a buffer. The buffer solutions are placed onto 96 well trays and fed mechanically through any of the high-throughput HPLCs available on the market. In a preferred embodiment, latex can be very rapidly screened utilizing isocratic ultra-high performance liquid chromatography (UPLC).

A very rapid and efficient screening method is desirable to test sufficient plants for finding an advantageous mutation. Suitable alkaloid screening methods are for instance described in WO 2009/143574 and WO 2009/109012. Furthermore, by using UPLC apparatus with a very sensitive UV detector (e.g., a Waters Acquity UPLC) it is possible to quantify very low levels of alkaloid, meaning that even very small plants can be tested. Additionally, very rapid screening (e.g., 0.8 minute) of each plant can allow over 1000 samples to be analysed daily. As a result, the entire screening process may be conducted quickly.

Plants identified by the screening process to have an altered alkaloid profile of interest are grown further and examined in more detail. According to a preferred procedure herein, a second sample is taken from about 3% of plants to clarify or confirm the results of the initial screen. A more precise gradient UPLC method can then be used to obtain more accurate peak identification and quantification. Plants confirmed to have the desired alkaloid profile are transplanted to 200 mm (approx. 8 inch) pots for growing to maturity.

As used herein, the term “poppy straw” or “straw” is to be taken to mean the straw material obtained when the mature poppy capsules of a Papaver somniferum plant are collected, and threshed to remove the seeds to form a straw.

As used herein, the term “opium” is to be taken to mean the air-dried, milky exudation (i.e., the latex) from incised, unripe poppy capsules of a Papaver somniferum plant.

As used herein, the term “concentrate of poppy straw” or “CPS” is to be taken to mean the material arising when poppy straw has entered into a process for the concentration of its alkaloids in either liquid, solid or powder form which contains the phenanthrene alkaloids of the opium poppy.

As used herein, the phrase “stand of Papaver somniferum” or “stand of stably reproducing Papaver somniferum” or the like, refers to a group of two or more Papaver somniferum plants or stably reproducing Papaver somniferum plants located together. Typically, the stand will comprise at least 3, 4, 5, 6, 7, 8, 9, 10, 11, 12, 13, 14, 15, 16, 17, 18, 19, or 20 or more Papaver somniferum plants located together e.g., 30, 40, 50, 60, 70, 80, 90 or 100 or more of the plants. Typically, the plants are grown or growing in a field exposed to ambient environmental conditions.

As used herein, the term “alkaloid combination” is to be taken to refer to a particular combination of alkaloids wherein one or more of the alkaloids differs in content or proportion in the poppy straw of latex of a plant of P. somniferum of the invention relative to one more other of the alkaloids of the combination compared to the wild-type or parent P. somniferum from which the plant is derived. In particularly preferred embodiments, the alkaloid combination can comprise morphine, codeine and thebaine, or morphine, oripavine, codeine and thebaine. In further embodiments of the present invention, the alkaloid combination may comprise one or more additional alkaloids as may be selected from the group consisting of, codeinone, neopinone, protopine, laudanine, laudanosine, salutaridine, reticuline, papaverine and noscapine, in addition to morphine, codeine, thebaine and when included, oripavine.

A “stably reproducing” Papaver somniferum poppy plant as described herein refers to a poppy plant that is stably reproducing as required to plant and harvest seed from poppy crops over multiple generations where each generation would be suitable, without seed selection, for commercial planting of a field crop or stand of plants exhibiting the desired alkaloid characteristic(s) (e.g., an altered alkaloid profile as described herein). Further, a stably reproducing poppy plant in accordance with the invention has the desired alkaloid characteristics as described herein, and when self-pollinated, or cross pollinated by a plant with the same genes controlling alkaloid content, produces a subsequent generation of plants which all have the same genetic potential to substantially have the same desired alkaloid characteristics as the parent plant. Moreover, in the absence of pollination with pollen from other chemotypes (e.g., conventional morphine accumulating plants), the line will continue to produce similar plants over multiple generations, without the need for selection to maintain the desired alkaloid characteristic.

As above, the term “stably heritable” as used herein is to be taken to mean the Papaver somniferum produces a subsequent generation of plants which all have the same genetic potential to substantially have the same alkaloid characteristics as the parent plant, when the plant is self-pollinated, or cross pollinated by a plant with the same genes controlling alkaloid content as described above. As above, in the absence of pollination with pollen from other chemotypes (e.g., conventional morphine accumulating plants), the line will continue to produce similar plants over multiple generations, without the need for selection to maintain the specified alkaloid characteristic.

As used herein the term “trait” is to be taken to mean a distinct heritable phenotypic characteristic. The desired trait(s), once established are consistently inherited by substantially all the progeny. To maintain the desired traits, care should be taken to prevent cross-pollination with normal plants unless such cross-pollination is part of a controlled breeding program.

Examples of desired trait(s) and/or alkaloid characteristics of Papaver somniferum plants embodied by the invention which can be passed on to future generations (e.g., progeny and further descendent plants thereof) include a) a high content of an alkaloid of interest (e.g., thebaine, codeine or morphine) as described herein in poppy straw, latex and/or opium, b) a high content of the alkaloid of interest relative to one or more confounding alkaloids in the poppy straw, latex and/or opium, c) a decrease in one or more confounding alkaloids relative to the alkaloid of interest, and d) a lighter leaf colour as described herein in combination with a), b) or c).

Typically, a plant embodied by the invention has a trait for lighter leaf colour as described herein that is controlled by a single recessive gene, wherein the trait is associated with at least one of an increase in content of the alkaloid of interest and a reduction in content of at least one confounding alkaloid relative to the content of the alkaloid of interest, in the poppy straw, latex or or opium of the plant.

For example, a plant of P. somniferum embodied by the invention with a thebaine chemotype may have an increased absolute content of thebaine in poppy straw, latex or opium of the plant as well as an increase in the proportion of thebaine relative to one or more confounding alkaloids selected from e.g., morphine, codeine and oripavine. The relative increase in the proportion of the thebaine may be associated with a lesser increase in the content of the confounding alkaloid(s) or as a result of a decrease in the content of the confounding alkaloid(s) in the poppy straw, latex or opium compared to the wild-type plant.

The absolute content of a desired alkaloid (e.g., morphine, thebaine etc) in poppy straw or latex of a plant in accordance with the invention may be increased by up to 10% or 15% or more on a w/w basis compared to the content of the alkaloid in the poppy straw or latex of the wild-type plant. That is, the alkaloid may be increased by 1%, 2%, 3%, 4%, 5%, 6%, 7%, 8%, 9%, 10%, 11%, 12%, 13%, 14% or 15% w/w or more.

Similarly, the proportion of an alkaloid relative to another alkaloid or combination of alkaloids in the alkaloid profile of a plant of P. somniferum as described herein may be altered (i.e., increased or decreased) by up to 10% or more on a w/w basis compared to the wild-type plant. That is, the proportion of the alkaloid may be altered by 1%, 2%, 3%, 4%, 5%, 6%, 7%, 8%, 9%, 10% w/w or more

In at least some embodiments of a plant in accordance with the invention having a codeine chemotype, the plant will have one or both of an increased absolute codeine content and an increased proportion of codeine relative to one or more of morphine, oripavine and codeine in its poppy straw or latex compared to the wild-type plant.

In at least some embodiments of a plant in accordance with the invention having a thebaine chemotype, the plant will have one or both of an increased absolute thebaine content and an increased proportion of thebaine relative to one or more of morphine, oripavine and codeine in its poppy straw or latex compared to the wild-type plant. In at least some embodiments, the poppy straw or latex of the plant of the invention may contain essentially no morphine, oripavine and/or codeine.

In at least some embodiments of a plant embodied by the invention having a morphine chemotype, the plant will have one or both of an increased absolute morphine content and an increased proportion of morphine relative to one or more of oripavine, codeine and thebaine in its poppy straw or latex compared to the wild-type plant.

In at least some embodiments, the plant screened may have a noscapine chemotype. In such plants, the proportion of noscapine may be evaluated relative to one or more other phthalideisoquinoline alkaloids (e.g., those in the noscapine biosynthesis pathway) and/or benzylisoquinoline alkaloids such as (S)-reticuline, laudanine, morphine, codeine and/or thebaine.

In at least some embodiments, the screening of a P. somniferum plant to determine whether or not the plant has an altered alkaloid profile as described herein may comprise evaluating the leaf colour of the plant as described herein and/or analysing dry poppy capsule or poppy straw material, or latex of the plant (e.g., latex collected from a leaf or immature poppy capsule) as described above for the content or proportion of one or more alkaloids relative to one or more other alkaloids compared to the parent or wild-type plant, such as by ultra-performance liquid chromatography (UPLC) as described herein. In preferred embodiments, the alkaloid(s) screened may be selected from the group consisting of benzylisoquinoline alkaloids and phthalideisoquinoline alkaloids, although other isoquinoline alkaloids of P. somniferum are not excluded.

Typically, the benzylisoquinoline alkaloids screened will be selected from the group consisting of morphine, codeine, oripavine, thebaine and noscapine.

Most typically, the benylisoquinoline alkaloids screened will be selected from morphine, codeine, oripavine and thebaine.

Phthalideisoquinoline alkaloids screened in accordance with methods as described herein will be selected from noscapine, (S)-tetrahydrocolumbamine, (S)-canadine, sanguinarine, and intermediate alkaloids in the sanguinarine biosynthesis pathway from (S)-scoulerine, such as (S)-cheilanthifoline. Most typically, of the P. somniferum alkaloids in this class, the one or more alkaloids screened in accordance with a method of the invention will comprise noscapine.

Screening for a plant embodied by the invention may also comprise screening for reduced expression or activity of magnesium chelatase activity of the enzyme itself such as via a suitable enzyme activity or other assay.

Still further, screening for reduced expression or activity of magnesium chelatase or for the identification of a plant embodied by the invention may comprise analysing nucleic acid of the plant for the presence of a nucleic acid molecule associated with reduced expression or activity of magnesium chelatase in the plant, the presence of the nucleic acid molecule being indicative that the plant exhibits the altered alkaloid profile. The nucleic acid molecule will typically comprise a nucleotide sequence comprising at least one mutation or polymorphism in a gene associated with the reduced expression or activity of the magnesium chelatase in the plant. The gene may, for example, encode a mutant subunit of the enzyme (e.g., the CHLI-A or other subunit) as described above.

In at least some embodiments in accordance with the invention, one or more mutations may be introduced into an untranslated regulatory region of the gene (e.g., in the 5′ untranslated region of the gene such as the promoter), whereby expression of the gene is thereby reduced. Alternatively, the mutation(s) may be introduced into a coding region of the gene whereby although the gene is expressed, the function of the encoded polypeptide product is deficient whereby the activity of the magnesium chelatase enzyme is thereby reduced. In particularly preferred embodiments, the mutation is a single nucleotide polymorphism (SNP) although any mutation or combination of mutations resulting in reduced expression or activity of magnesium chelatase resulting as described herein is expressly encompassed. Various such possible gene modifications will be apparent to persons in the field of endeavour to which the present invention relates.

The analysis of the nucleic acid associated with reduced expression or activity of magnesium chelatase may comprise sequencing nucleic acid isolated from the plant utilising any appropriate sequencing method. Such protocols may involve DNA isolation followed by polymerase chain reaction (PCR) amplification of the target nucleic acid gene sequence and subsequent sequencing of the amplified product using Sanger or next-generation sequencing (NGS). Genetic polymorphisms within the genetic sequence can then be identified through bioinformatic analyses of the resulting gene sequencing data. Other PCR-based protocols can also be used to identify mutations (e.g., polymorphisms) in plant individuals and or be used for high-throughput genotyping. For example, high resolution melt (HRM) analysis is a new generation of mutation scanning and genotyping technology. It utilises intercalating fluorescent dyes to bind to the double-stranded DNA fragments created during PCR. Following PCR, the amplicon DNA is then heated so that the double-stranded DNA separates (i.e., ‘melts’) and the intercalating fluorescent dye is released, thereby resulting in a loss of fluorescence. As the melting temperature of an amplicon is dependent on its' DNA base composition, polymorphisms result in different melting temperatures and therefore measurable differences in fluorescence between individuals having different sequences when quantitively measured in real-time throughout the PCR-based heating process can be detected (Li, Y-D et al (2010) A cost-effective high-resolution melting approach using the EvaGreen Dye for DNA polymorphism detection and genotyping in plants. Journal of Integrative Plant Biology, 52(12), 1036-1042; doi: 10.1111/j.1744-7909.2010.01001.x). Genotyping assays such as TaqMan SNP assays can be further used to analyse genetic polymorphisms through utilising fluorescent probes in quantitative PCR (Holland PM et al (1991) Detection of specific polymerase chain reaction product by utilizing the 5 ′—3′ exonuclease activity of Thermus aquaticus DNA polymerase. PNAS, 88(16), 7276-7280) and provides a further example by which nucleic acids associated with reduced expression or activity of magnesium chelatase as described herein may be evaluated.

As used herein, the “M1 population” is the seeds and resulting plants exposed to a mutagenic agent, while “M2 population” is the progeny of self-pollinated M1 plants, “M3 population” is the progeny of self-pollinated M2 plants, “M4 population” is the the progeny of self-pollinated M3 plants, and generally “Mn population” is the progeny of self-pollinated Mn-1 plants.

The trait(s) of reduced expression or activity of magnesium chelatase activity and lighter leaf colour phenotype as described herein can be transferred into Papaver somniferum lines having other characteristics (e.g., a different chemotype or different alkaloid profile in its poppy straw or latex, different height, early or late maturity, or disease resistance etc.) by cross pollinating a plant embodied by the invention with the second parent plant, collecting F1 seed, growing a F1 plant which is allowed to self-pollinate, and collecting the F2 seed. The F2 seed may then be grown, and individual plants that have the lighter leaf colour and altered alkaloid profile or other alkaloid phenotypic characteristic(s) as described herein along with other of the desired characteristic(s) e.g., disease resistance, may be selected according to methods described herein. Further selection can then be undertaken if desired in the F3 and/or subsequent generations in order to produce highly uniform plant lines. A skilled operator will be able to apply variations to this method as well known in conventional plant breeding.

Conducting test crosses with plants of known genotype can provide information regarding the genetic changes introduced through mutation. The characteristics of the F1 generation produced by crossing to a normal parent will indicate whether a trait inherits as a recessive or dominant gene. Self-pollinating the F1 plants and determining the phenotypes of the subsequent F2 population of plants will provide information regarding the numbers of genes responsible for particular characteristics.

As used herein, the term “descendent” plant is meant a Papaver somniferum plant which is the progeny of a plant embodied by the invention or is derived from a plant embodied by the invention such as a granddaughter plant, great granddaughter plant and the like, or a plant as may be obtained by cross-pollinating a plant embodied by the invention (or e.g., a progeny plant thereof) with another Papaver somniferum poppy line having desirable trait(s) of interest, testing the progeny at the F2 or F3 or subsequent generations, and selecting progeny on the basis of one or both of exhibiting a lighter leaf colour phenotype and an altered alkaloid profile

In preferred embodiments of the invention, seed from Papaver somniferum which upon harvesting of their capsules produce a poppy straw containing thebaine as the predominant alkaloid in the alkaloid combination of morphine, codeine, thebaine and oripavine, or alternatively, which upon the drying of latex from their immature poppy capsules will yield an opium containing thebaine as the predominant alkaloid of the alkaloid combination, is used to provide a plant embodied by the present invention.

Typically, the poppy straw of the Papaver somniferum parent plant will contain thebaine and oripavine constituting about 50% by weight or greater of the alkaloid combination comprising morphine, codeine, thebaine and oripavine. Preferably, thebaine and oripavine will constitute about 50%, 55%, 60%, 65%, 70%, 75%, 80%, 85%, 90%, or 95%, by weight or greater of the alkaloid combination.

In preferred embodiments in which the plant embodied by the invention has a thebaine chemotype, the plant typically will contain substantially no morphine or codeine, and/or substantially no oripavine, in the alkaloid combination of morphine, codeine, thebaine and oripavine in poppy straw, opium or latex of the plant.

The term “substantially no”, when referring to morphine, codeine, thebaine, or oripavine means that each specified alkaloid respectively constitutes less than 1% by weight, preferably, less than 0.5% by weight, more preferably, less than 0.3% by weight, and most preferably, from 0% to 0.2% by weight of the alkaloid combination comprising morphine, codeine and thebaine, and most preferably, the alkaloid combination comprising morphine, codeine, thebaine and oripavine, of the poppy straw, concentrate of poppy straw, latex or opium.

Most preferably, the term “substantially no” in the context of oripavine means oripavine constitutes less than 0.6% by weight, preferably less than 0.5% by weight, more preferably less than 0.4% by weight and most preferably, between 0% and 0.2% by weight of the alkaloid combination of morphine, codeine, oripavine and thebaine.

Stably reproducing Papaver somniferum parent plants suitable for use in providing a plant having a stably heritable chemotype in accordance with a method of the present invention are, for example, described in WO 98/0202033 and WO 2009/109012 both in the name of Tasmanian Alkaloids Pty Ltd, the entire contents of which are incorporated herein in their entirety by cross-reference. The high thebaine plants described in WO 2009/109012 are believed to be the result of two independent genetic changes in the plants, one genetic change controlling the accumulation of thebaine and oripavine compared with morphine and codeine, and the second genetic change controlling the accumulation of thebaine compared with oripavine. More particularly, the two independent genetic changes were provided by mutation of a first gene blocking thebaine from being converted to neopinone, and oripavine from being converted to morphinone (as exemplified by the TOP 1 mutation; Millgate et al., Morphine-pathway block in top1 poppies. Nature, Vol. 431, 413-414, 2004), and a further mutation blocking a pathway between thebaine and oripavine, see the metabolic pathway set out in Scheme 1 below (modified from Beaudoin GAW and Facchini PJ (2014), Benzylisoquinoline alkaloid biosynthesis in opium poppy. Planta, 240,19-32; DOI 10.1007/s00425-014-2056-8). As shown in Scheme 1, P. somniferum is postulated to have two biosynthetic pathways from thebaine to morphine. Pathway A via neopinone, codeinone and codeine was proposed by Parker, H. I., J. Am. Chem. Soc., 94, 1276-1282 (1972). Pathway B via oripavine and morphinone was proposed by Brochmann-Hanssen, E., Planta Med., 50, 343-345 (1984).

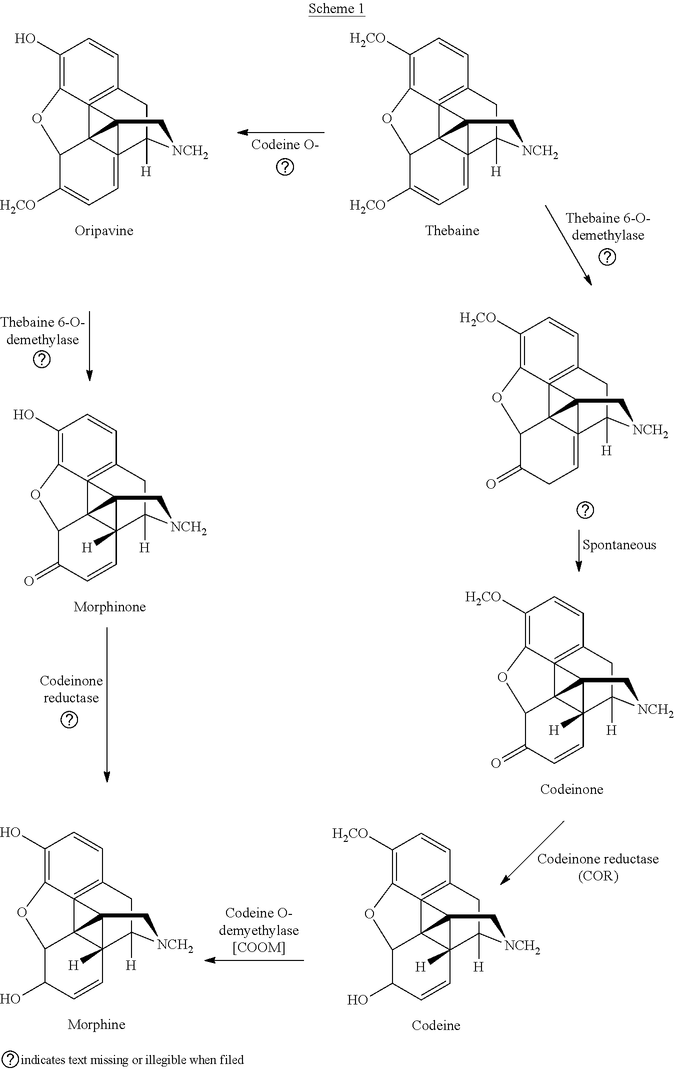

Seed of a P. somniferum (‘Ted’) plant line having a thebaine chemotype as described in WO 2009/109012 and which is useful as a second parent plant for crossing with a plant embodied by the invention for generating new plants in accordance with the invention has been deposited under the provisions of the Budapest Treaty with the American Type Culture Collection, 10801 University Boulevard, Manassas, Va. 20110-2209, on 20 Mar.2008 under ATCC® Accession No. PTA-9109.

The term “% w/w” or “% w/w basis” or the like as used herein in the context of the content of a specified alkaloid relative to poppy straw is meant the content of the alkaloid in poppy straw obtained from mature, field dried Papaver somniferum.

Plants contain a variety of pigments that contribute to both prominent visual features (e.g., flower colour) and important physiological processes. One of the most well-known classes of plant pigments are the chlorophylls (e.g., chlorophyll a and b). These pigments play essential roles in photosynthesis including the capture and harvesting of light energy from the sun. Humans recognize pigment colour by perceiving the visible light (i.e., wavelengths between ˜390 to ˜700 nm) which is reflected or transmitted by the pigment. For example, the characteristic green colour of chlorophylls can be explained by the fact that chlorophylls absorb light in the violet-to-blue and red light regions, leaving a considerably wide gap in the absorption spectrum known as the ‘green window’ (Chen, M. (2014) Chlorophyll modifications and their spectral extension in oxygenic photosynthesis. Annual Review of Biochemistry, 83, 317-340). The reflectance of visible light in this so-called green window give chlorophylls their green colour.

The importance and prevalence of chlorophylls explains the abundance of green-coloured tissues in plants. However, many other non-green plant pigments also occur and similarly provide vital physiological roles. A group of pigments called carotenoids confer yellow-to-red coloration to flowers and fruits. Along with chlorophylls, carotenoid pigments constitute an essential component of the photosystem light-harvesting complexes involved in photosynthesis (Tanaka, Y., Sasaki, N. and Ohmiya, A. (2008) Biosynthesis of plant pigments: anthocyanins, betalains and carotenoids. The Plant Journal, 54, 733-749). Carotenes are a class of the carotenoid pigment family and include β-carotene; a major carotenoid pigment in higher plants (Hopkins, W. G. and Winer (2004) Introduction to Plant Physiology (3^{rd} ed.) Wiley and Sons (MA, USA)). Whilst carotenoid pigments including β-carotene can serve as accessory pigments in the capture of light energy, their principal function is that of an anti-oxidant, preventing photooxidative damage to the chlorophyll molecules within the chloroplast (Raven, P. H., Evert, R. F. and Eichhorn, S. E. (1999) Biology of Plants (6^{th} ed.) W. H. Freeman and Company (NY, USA)). Further, the carotenoid pigments violaxanthin, antheraxanthin and zeaxanthin, also present in chloroplasts, function in a process known as the xanthophyll cycle which serves to dissipate excess energy and thereby provide photoprotection (Hopkins, W. G. and Winer (2004), vide supra). Although present in leaf tissues, the colours of carotenoid pigments are generally masked by the more abundant chlorophylls. However, carotenoids and other plant pigments may become visible in plant leaves under certain conditions. For example, during autumn chlorophyll pigments are degraded in leaves of deciduous plant species. During this leaf senescence process the more stable carotenoid pigments are revealed, resulting in the characteristic orange and yellow foliage colours of autumn (Hopkins and Hüner 2004, vide supra). Alternatively, other species may produce brilliant red foliage during autumn (e.g., Quercus rubra; red oak) due to the accumulation of anthocyanin pigments in their leaves (Lee, D. W. and Gould, K. S. (2002) Why leaves turn red. American Scientist, 90, 524-531). Anthocyanins belong to another major class of plant pigments called flavonoids. These phenylpropanoid secondary metabolites have a wide colour range, ranging from pale-yellow to blue. Notably, the anthocyanins are responsible for the orange to blue colours found in many flowers, leaves, fruits, seeds and other tissues (Tanaka et al. 2008, vide supra). Other flavonoids, such as the flavonols, are commonly found in leaves and along with flavones are very pale-yellow. Whilst these pigments can be mostly invisible to the human eye, their ultra-violet (UV) absorbing properties provide colour and patterns that serve to attract insect pollinators in addition to protecting against UV damage (Winkel-Shirley, B. (2002) Biosynthesis of flavonoids and effects of stress. Current Opinion in Plant Biology, 5, 218-223; Tanaka et al. 2008, vida supra).

As described herein, in at least some embodiments of the invention, plants having a noticeably lighter leaf colour than the parent plant from which they were derived were found to nevertheless have an altered alkaloid profile compared to the unmodified parent plant, the colour ranging from a visually lighter green-yellow to very light green-yellow coloured leaves. To evaluate the colour of the leaves, spectroscopic measurements were taken to obtain three-dimensional (3D) colour coordinates. Chromaticity coordinates were then calculated from the spectrophotometer results in order to reduce the dimensionality of the data and to obtain dominant wavelength values for each plant line evaluated. The dominant wavelength, as measured in nanometers (nm), is a measure of the hue of an object's colour and is used herein to describe leaf colour. Methods of colour measurement and colour description as described herein are well known in the art and are described in colourimetry texts including, for example, Wyszecki, G., & Stiles, W. S. (1982), Color Science: concepts and methods, quantitative data and formulae (2nd Ed.; New York: Wiley).

The dominant wavelength values of the measured leaves of plants described herein corresponded to the green-yellow spectral wavelength region. Thus, the respective plants can be described as all having green-yellow leaves, and plants having lighter coloured leaves than the parent plant may be described as having e.g., light green-yellow leaves, lime green, or for instance, very light green-yellow leaves, compared to the parent plant. Hence, the term “green-yellow” as used herein in the context of the leaf colour of a plant embodied by the invention and/or parent plant, is to be taken to mean green-yellow in the context of the green-yellow colour spectrum.

Plants embodied by the invention which are grown in the field (and so are exposed to ambient weather conditions during their development) typically have leaves that are markedly lighter in colour than if those plants were grown in a planthouse. As such, plants of the invention that are field grown will typically have a dominant wavelength value that is greater than that if the plants were planthouse grown plants. The term “planthouse” is used herein herein to refer to either a plastic covered “polyhouse” or to a greenhouse.

Typically, a plant of Papaver somniferum having a lighter leaf colour embodied by the invention as described above will have green-yellow leaves exhibiting a dominant wavelength value in a range of from about 561 nm to 568 nm or greater, but not exceeding 570 nm. In other embodiments, plants embodied by the invention, or identified in accordance with embodiments of the invention, predominantly have leaves exhibiting a dominant wavelength that is different to the dominant wavelength of the leaves of the parent plant. Typically, the dominant wavelength of at least a majority of the leaves of a plant of the invention is different to the dominant wavelength of at least the majority of the leaves of the parent plant.

The dominant wavelength value of a plant embodied by the invention may, for instance, be in a range of from about 561 nm to about 568 nm e.g., a dominant wavelength of about 562 nm, 563 nm, 564 nm, 565 nm, 566 nm, 567 nm, or 568 nm. It will also be understood that all ranges with the dominant wavelength identified above are expressly encompassed. For instance, a plant embodied by the invention having a lighter leaf colour may have a green-yellow leaf colour exhibiting a dominant wavelength in a range of from about 561 nm to about 570 nm, from about 561 nm to about 569 nm, from about 561 nm to about 568 nm, from about 562 nm to about 568 nm, from 562 nm to about 567 nm, or from 563 nm to about or 566 nm. A method for the measurement of the dominant wavelength is exemplified further below.

Typically, the dominant wavelength is determined by reflective spectrophotometry on the adaxial surface of healthy, leaves using D65 illumination, wherein healthy leaves are characterised as leaves being free from visible signs of disease, senescence, nutrient deficiency/toxicity, and other forms of stress (e.g., temperature-, water, or herbivory-related stress).

The leaf colour can be evaluated at any stage up until maturity of a plant embodied by the invention and compared with the leaf colour of the parent plant at the same stage of development, such as running up, late running up, bud in apex, early hook, hook, mid-hook, upright bud and first flower stages. In particularly preferred embodiments, leaf colour is assessed in the early hook or hook stages and more preferably in the early to mid-hook growth stages. As the leaf colour difference in light-leaf colour plants embodied by the invention is more pronounced in field grown plants compared to field grown parent plants, it is desirable that the evaluation of leaf colour is undertaken on field grown plants. Likewise, it is preferable that leaf colour comparisons be made between plants grown in the same location and/or under the same conditions. The determination of conditions for growing plants for purposes of leaf colour comparisons are well within the expertise of a person of ordinary skill in the art. Conditions suitable for growing plants in a planthouse for the purpose of leaf colour comparisons are, for instance, further described below.

Whilst dominant wavelength is exemplified herein as a measure of the leaf colour, other parameters may be used to evaluate leaf colour of a plant embodied by the invention relative to a parent plant as described herein. For example, other methods for measuring leaf colour may include evaluating the content of one or more pigments in leaves responsible for the leaf colour a plant, such as cholorophyll, (e.g., cholorophyll a and/or chlorophyll b), accessory pigments such as one or more carotenoid(s), anthocyanins, and mixtures of the foregoing, and all suitable alternative methods are expressly encompassed.

Typically, a plant embodied by the invention has a reduced leaf pigment content comprising a reduced level of at least one of chlorophylls and carotenoids in the leaves of the plant.

Most typically, the reduced level of chlorophylls comprises a reduced level of both chlorophyll a and chlorophyll b. In at least some embodiments, the level of chlorophylls may be reduced by least 10% by weight. In some embodiments, the level of chlorophylls may be reduced by up to 20% by weight or even more (e.g., by 10%, 11%, 12%, 13%, 14%, 15%, 16%, 17%, 18%, 19%, 20% or even by 25%, 30%, 35%, 40%, 45%, or 50% by weight or more).

The reduced level of carotenoids will typically comprise a reduced level of at least one of lutein and β-carotenoid.

Despite having a lighter leaf colour on the adaxial surface of leaves wherein the lighter leaf colour is associated with a modified alkaloid profile in poppy straw or opium, plants in accordance with the invention can nevertheless be healthy, viable plants exhibiting good vigour with no symptoms of disease or nutrient deficiency.

As used herein, the term “field grown” is to be taken to mean plants grown in situ from seed sown in the field and plants that are grown to maturity in the field from transplanted seedlings raised from seed e.g., in a planthouse.

In at least some embodiments, a plant of the present invention may be asexually reproduced, including via methods such as tissue culture.

Recovering the thebaine, codeine other alkaloid of interest from dried poppy straw, opium or concentrate of poppy straw is a process well established in the art. Thebaine, for instance, has been extracted from P. somniferum either as a part of the process of extracting morphine and codeine, or more recently as part of the process of extracting thebaine and oripavine.

For example, the poppy straw can be treated with a small amount of lime and water to soften the capsules and to form a free base of the alkaloids. Countercurrent extraction of the softened straw with methanol, ethanol or other suitable solvent forms a solvent/water extract or “miscella” containing the alkaloids, with morphine at a concentration of about 1 g/L where the straw is from standard Papaver somniferum. The volume of the miscella is reduced about 30× under vacuum to produce an aqueous concentrate. Any thebaine can be extracted from the aqueous concentrate using one or more liquid/liquid partitioning extraction steps using suitable solvent(s) (e.g., toluene or xylene), adjusting pH for the best separation of thebaine. Codeine remains in the aqueous phase and Codeine CPS can be precipitated from the aqueous phase by pH adjustment.

An alternative means of producing alkaloids is to grow plant cells or plant organs such as shoots or roots in culture, and all such methods are expressly encompassed herein. Cell culture or organ culture are means of producing alkaloids without being subject to the vagaries of climate and other uncertainties associated with crop production. The general methods of establishing cell cultures, root cultures and shoot cultures for the purpose of alkaloid production are provided by M. F. Roberts, Production of alkaloids in plant cell culture. In Alkaloids, Biochemistry, Ecology, and Medicinal Applications, Edited by Roberts and Wink, Plenum Press, New York 1998, pages 159-197, the contents of which is hereby incorporated by reference in its entirety. The first step in producing cell cultures is to establish growth of callus. One way of achieving this for Papaver somniferum is provided by Chitty et al. (2003)., Genetic transformation in commercial Tasmanian cultivars of opium poppy, Papaver somniferum L., and movement of transgenic pollen in the field. Functional Plant Biology 30: 1045-1058. In this method, seeds are surface sterilized by washing for 30-60 seconds in 70% ethanol, then in 1% (w/v) sodium hypochlorite solution plus 1-2 drops of autoclaved Tween 20 for 20 minutes with agitation. Seeds are then rinsed three to four times in sterile distilled water, or until no smell of bleach remains, and placed on B50 agar medium (Gamborg et al. 1968 Nutrient requirements of suspension cultures of soybean root cells. Experimental Cell Research 50, 151-158). Dishes are sealed with parafilm and imbibed at 4° C. for 24 to 48 hours. Seeds are germinated at 24° C. in a 16 hour light-8 hour dark cycle. Hypocotyls are excised from seedlings after 7-8 days of culture, cut into 3-6 mm pieces (usually 1-3 explants per seedling) and placed onto callusing media. Culture media consists of B50 macronutrients, micronutrients, iron salts and vitamins (Gamborg et al. 1968) and sucrose at 20 g/L. pH can be adjusted with 1M KOH to pH 5.6 and 0.8% Sigma agar (A1296) can be used as a gelling agent.

All media should be autoclaved at 121° C. for 20 minutes. B50 medium contains no growth regulators and is used to germinate seeds aseptically, maintain embryogenic callus, and regenerate shoots and plantlets. Callusing Medium (CM) is B50 medium plus 2,4-dichlorophenoxy acetic acid (2,4-D) at 1 mg/l, added prior to the medium being autoclaved.

To generate a cell suspension culture (method from Staba et al. 1982, Alkaloid production from Papaver tissue cultures, Journal of Natural Products, 43,256-262), callus cultures can be transferred into 125 mL Erlenmeyer flasks containing 25 mL of liquid RT medium (Khanna and Khanna 1976, Ind J Exp Biol 14,628) supplemented with either 5 ppm BA for the growth of shoots or 0.1 ppm 2,4-D for the development of cell suspensions. Cultures can be grown at 28° C. on an orbital shaker (78 rpm) with 15 hours of light per day. In particular, cell cultures can be grown as a batch culture where the cells multiply in a liquid medium which is being continuously agitated to maintain the cells as small aggregates and to maintain oxygen levels. Typically, after the initial inoculation there is a lag phase, followed by an exponential growth phase, which is then followed by a stationary phase where the growth becomes limited by lack of some components of the medium. Often, secondary plant products such as alkaloids are accumulated while the culture is in the stationary phase. For some products, alkaloid production can be induced by adding elicitors such as fungal cell extracts. There are also systems of continuous or semi-continuous culture where fresh medium is added either continuously or semi-continuously while cells or media are likewise removed for alkaloid recovery. Critical to the success of any cell culture system is the establishment of high yielding cell lines. Generally, selection is required to select individual plants, or individual cell cultures that produce the required alkaloid. For the production of codeine, a rapid HPLC or UPLC method such as those described in this application could be modified to test cell lines for codeine production.

Techniques such as root culture including hairy root culture where roots are transformed with Agrobacterium rhizogenes may also be a viable means of producing codeine in culture. A method for transformation of Papaver somniferum cultures with A. rhizogenes is, for instance, described in Yoshimatsu and Shimomura (1992), Transformation of opium poppy (Papaver somniferum L.) with Agrobacterium rhizogenes MAFF 03-01724. Plant Cell Reports 11,132-136. A person skilled in the art of cell and organ culture would also be able to envisage other means of growing plant cells derived from plants embodied by the present invention in order to produce codeine.

Methods for sampling leaf latex, and measuring the content of codeine, thebaine, morphine, oripavine and other alkaloids as described herein in poppy straw, latex, opium and concentrate or of poppy straw are well known to the skilled person, see for instance WO 2009/143574 and WO 2009/109012.

The invention is further described below with reference to a number of Examples. The Examples are not intended and should not be construed as limiting the invention in any way.

### EXAMPLE 1

### EMS Mutagenesis Treatment of Seed of a Papaver Somniferum Line Commercially Grown For The Production of Codeine

The seed of a stably reproducing Papaver somniferum “Tasman” parent line (PW08-2308) which produces codeine as the predominant alkaloid and relatively low levels of thebaine and essentially no morphine or oripavine as described in WO 2009/143574 was subjected to EMS mutagenesis treatment.

In brief, 2×2 g lots of seed from the commercially grown Papaver somniferum “Tasman” line (PW08-2308) were utilised in the present study. Each 2 g seed lot was placed in a 15 cm×15 cm square of porous mesh curtain material and the corners of the material were tied together to form a pouch. The bags of Tasman seed were placed in separate 250 mL flasks filled with chilled (4° C.) 100 mM phosphate buffer (pH 7). The flasks were sealed with a rubber stopper and placed in a refrigerator at 4° C.) where they were left overnight.

The next day the bags of seed were removed from the phosphate buffer solution, and a new solution of 0.7% EMS (ethyl methanesulfonate) in phosphate buffer was prepared by adding 1.75 mL of EMS to 250 mL phosphate buffer. The bags of seed were added to the EMS solution, the flask was capped and the solution stirred on a magnetic stirrer (without heat). Seed was treated in this way for 5 hours.

At the end of each treatment period the bags of seed were removed from the EMS solution and rinsed under running water for 30 minutes. The seed was then left overnight in 200 mL distilled H₂O at 4° C. The next day seeds were again rinsed under running water for 30 minutes, spread thinly on a layer of tissue paper and left to air-dry for 2 hours to facilitate ease of handling when sowing. The air-dried mutagenized M1 seed was immediately sown in 14 cm diameter pots filled with potting soil (a 50:50 mix of coarse and composted pine bark, with Osmocote™ slow release fertiliser added). Seeds were sown at the rate of about 10 per pot and 96 pots of each line/EMS treatment combination were sown. After sowing the seed was covered with a fine layer of vermiculite. Watering was via overhead sprinklers for the first 14 days followed by drip irrigation through to maturity. Plants were fertilised by weekly application of a liquid fertiliser. The M1 generation was grown in an enclosed (plastic covered) planthouse under natural day lengths in Westbury, Tasmania, Australia. Individual M1 plants were self-pollinated through the use of a paper bag placed and secured over individual flower buds prior to anthesis.

At maturity, M2 seed was harvested from each M1 plant and placed in a separate labelled seed packet. An equal amount (0.08 g) of M2 seed was then taken from each packet to produce four bulk M2 seed samples.

Bulked M2 seed was sown in a field breeding nursery (Weetah, Tasmania, Australia) on 22 Sep. 2011. The seed was sown in 5 m long×1.6 wide plots, with 6 rows per plot, using a custom-built-trial-seed drill. Seventy plots of the Tasman/5 hr treatments were sown, with approximately 0.24 g of seed used to sow each plot. Basal fertilizer was incorporated into the soil at the time of sowing.

Plants were inspected regularly throughout development to screen for interesting and potentially useful phenotypes. At early stages of development plants with phenotypes of interest were marked with a flag to ensure they were examined further. After ‘running up’ all of these plants were tagged with a label that described their phenotype. All labelled phenotypic mutants were self-pollinated by placing and securing a paper bag over the primary flower bud prior to anthesis. In addition, 1200 ‘Tasman’ M2 plants, with normal phenotypes, were selected at random and self-pollinated by securing a paper bag over the primary flower bud prior to anthesis.

M3 seed from each of the M2 plants was harvested separately when plants were fully mature and dry (February 2012) and were assigned line numbers starting with EM3 or EM4.

M3 seed from ‘Tasman’ phenotypic mutant lines were grown in the planthouse during April 2012 to further examine their phenotype and bulk M4 seed for future field trials. Plants were grown 6-per-pot in 14 cm diameter pots in commercial potting soil, under a 16 hr photoperiod.

### EXAMPLE 2

### M3 Field Trials

### 1. M3 Alkaloid/Phenotype Screening Trial

A number of M2 mutant plants with interesting phenoytpes were identified in the M2 populations grown (Weetah, Tasmania, Australia) during the 2011/12 season as outlined in Example 1. Some of the phenotypes observed in the M2 field grown plants were not evident when the M3 generation was grown in the planthouse, and these M3 lines were excluded from further investigations. Overall, 982 M3 lines that appeared worthy of further investigation were advanced to field trials (Hagley, Tasmania, Australia) during the 2012/13 poppy growing season.

M3 ‘Tasman’ mutant lines produced in Example 1 were sown in one or more of three different field trials, to assess, various aspects of their alkaloid content, alkaloid profiles and/or phenotypes.

The first of these trials, the M3 alkaloid screening trial, was conducted in a paddock (Hagley, Tasmania, Australia) during the 2012/13 poppy growing season. Each M3 line was sown in a single, 5 m long row within a 5 m×1.8 m plot that contained six such rows. Multiple replicates of the ‘Tasman’ parent line were grown throughout the trial. A visual assessment of plant phenotype was conducted prior to flowering. Plants were allowed to mature and dry under field conditions, and all capsules from each row (each of the parent and M3 lines) were harvested for analysis of alkaloid content and profile. Capsules were weighed and, threshed to remove seed to produce poppy straw. The straw was then weighed and ground using a Retsch Grindomix GM 200 in preparation for alkaloid extraction. 2 g of the ground straw was placed in a plastic tube and suspended in 40 mL of extraction solution (consisting of 2% acetic acid and 10% ethanol in distilled water), then shaken on an orbital shaker for 90 minutes. 240 μL of extractant solution from each sample was filtered through a 0.45 μM Pall filter prior to the analysis of alkaloid content using a Waters Acquity ULPC® (Ultra High Performance Liquid Chromotography) system. Sample components were separated on a Waters Acquity UPLC BEH C18 Column (Part No. 186002352) by a gradient method using A. 2% acetic acid in water and B. acetonitrile (HPLC grade). The column temperature was maintained at 44° C. and the detector wavelength was set at 284 nm. Chromatographic peak areas were analysed and alkaloid content (% w/w) calculated using Empower software.

The results for each single replicate M3 line were compared to a mean value for the parent line (PW08-2308) included in the trial. Two other Papaver somniferum lines with a codeine chemotype, namely PW11-4063 and PW10-0149 derived from a breeding program, were included in the trial for comparison with the mutant M3 lines.

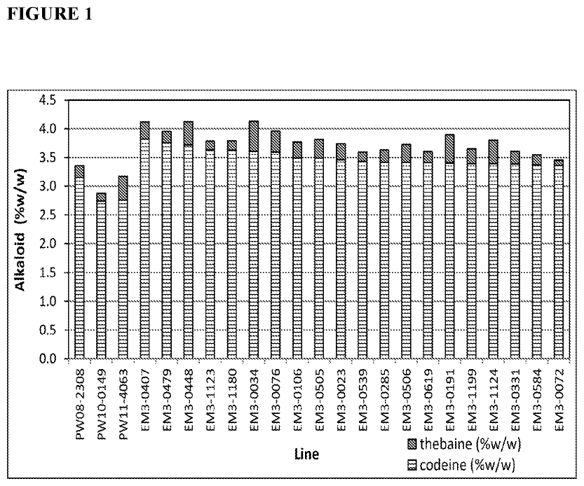

Of the 982 M3 lines screened, 80 M3 lines exhibited increased codeine content relative to the parent line (PW08-2308), and 139 M3 lines exhibited a thebaine/codeine ratio (T/C) lower than the parent line PW08-2308 (T/C of 0.06, that is a reduced amount of codeine relative to thebaine, the best lines being (EM3-0281, EM3-0538, EM3-0832 and EM3-0515, with a T/C of 0.01). No data was obtained for 68 M3 lines as a result of poor establishment in one section of the trial. Due to the high-vigour phenotype of the PW08-2308 parent line, M3 lines having mutations that affected plant health and vigour were very obvious. A total of 16 M3 lines were identified as showing very low vigour. Determined levels of codeine, thebaine, morphine and oripavine in selected M3 lines are shown in Table 1. A graph illustrating the codeine and thebaine content of the poppy straw from the best 20 codeine producing M3 lines is shown in FIG. 1

TABLE 1
Alkaloid content of poppy straw of selected P. somniferum M3 lines

| | | | | | | Thebaine/ | | | Traits |
|---|---|---|---|---|---|---|---|---|---|
| | | | | | TOTAL | Codeine | | TOTAL | improved |
| | codeine | morphine | oripavine | thebaine | MOCT | ratio | Codeine % | MOCT % | relative to |
| Line | (% w/w) | (% w/w) | (% w/w) | (% w/w) | (% w/w) | (T/C) | of PW08-2308 | of PW08-2308 | PW08-2308 |
| PW08-2308 | 3.15 | 0.00 | 0.00 | 0.20 | 3.36 | 0.06 | 100 | 100 | |
| PW10-0149 | 2.74 | 0.00 | 0.01 | 0.14 | 2.89 | 0.05 | 87 | 86 | |
| PW11-4063 | 2.76 | 0.00 | 0.00 | 0.42 | 3.17 | 0.15 | 87 | 94 | |
| EM3-0407 | 3.82 | 0.00 | 0.00 | 0.30 | 4.12 | 0.08 | 121 | 123 | codeine |
| EM3-0479 | 3.76 | 0.00 | 0.00 | 0.20 | 3.96 | 0.05 | 119 | 118 | codeine, T/C |
| EM3-0448 | 3.72 | 0.00 | 0.00 | 0.40 | 4.12 | 0.11 | 118 | 123 | codeine |
| EM3-1123 | 3.64 | 0.00 | 0.00 | 0.15 | 3.79 | 0.04 | 115 | 113 | codeine, T/C |
| EM3-1180 | 3.63 | 0.00 | 0.00 | 0.16 | 3.79 | 0.04 | 115 | 113 | codeine, T/C |
| EM3-0034 | 3.60 | 0.00 | 0.00 | 0.53 | 4.13 | 0.15 | 114 | 123 | codeine |
| EM3-0076 | 3.59 | 0.00 | 0.00 | 0.37 | 3.96 | 0.10 | 114 | 118 | codeine |
| EM3-0106 | 3.50 | 0.00 | 0.00 | 0.28 | 3.77 | 0.08 | 111 | 112 | codeine |
| EM3-0505 | 3.49 | 0.00 | 0.00 | 0.33 | 3.82 | 0.09 | 111 | 114 | codeine |
| EM3-0023 | 3.46 | 0.00 | 0.00 | 0.28 | 3.74 | 0.08 | 110 | 111 | codeine |
| EM3-0539 | 3.43 | 0.00 | 0.00 | 0.17 | 3.59 | 0.05 | 109 | 107 | codeine, T/C |
| EM3-0285 | 3.42 | 0.00 | 0.00 | 0.22 | 3.63 | 0.06 | 108 | 108 | codeine |
| EM3-0506 | 3.41 | 0.00 | 0.00 | 0.31 | 3.73 | 0.09 | 108 | 111 | codeine |
| EM3-0619 | 3.41 | 0.00 | 0.00 | 0.19 | 3.60 | 0.06 | 108 | 107 | codeine |
| EM3-0191 | 3.40 | 0.00 | 0.00 | 0.49 | 3.90 | 0.15 | 108 | 116 | codeine |
| EM3-1199 | 3.40 | 0.00 | 0.00 | 0.26 | 3.65 | 0.08 | 108 | 109 | codeine |
| EM3-1124 | 3.39 | 0.00 | 0.00 | 0.40 | 3.80 | 0.12 | 108 | 113 | codeine |
| EM3-0331 | 3.39 | 0.00 | 0.00 | 0.22 | 3.61 | 0.06 | 108 | 107 | codeine |
| EM3-0584 | 3.37 | 0.00 | 0.00 | 0.18 | 3.55 | 0.05 | 107 | 106 | codeine, T/C |
| EM3-0072 | 3.36 | 0.00 | 0.00 | 0.09 | 3.46 | 0.03 | 107 | 103 | codeine, T/C |
| EM3-1196 | 3.36 | 0.00 | 0.00 | 0.20 | 3.55 | 0.06 | 107 | 106 | codeine |
| EM3-0390 | 3.35 | 0.00 | 0.00 | 0.06 | 3.41 | 0.02 | 106 | 102 | codeine, T/C |
| EM3-0476 | 3.35 | 0.00 | 0.00 | 0.19 | 3.54 | 0.06 | 106 | 105 | codeine |
| EM3-0095 | 3.34 | 0.00 | 0.00 | 0.27 | 3.62 | 0.08 | 106 | 108 | codeine |
| EM3-1147 | 3.34 | 0.00 | 0.00 | 0.19 | 3.53 | 0.06 | 106 | 105 | codeine |
| EM3-1166 | 3.34 | 0.00 | 0.00 | 0.26 | 3.59 | 0.08 | 106 | 107 | codeine |
| EM3-0552 | 3.34 | 0.00 | 0.00 | 0.39 | 3.72 | 0.12 | 106 | 111 | codeine |
| EM3-0674 | 3.33 | 0.00 | 0.00 | 0.29 | 3.62 | 0.09 | 106 | 108 | codeine |
| EM3-0551 | 3.32 | 0.00 | 0.00 | 0.20 | 3.52 | 0.06 | 105 | 105 | codeine |
| EM3-1026 | 3.32 | 0.00 | 0.00 | 0.20 | 3.52 | 0.06 | 105 | 105 | codeine |
| EM3-1138 | 3.32 | 0.00 | 0.00 | 0.24 | 3.56 | 0.07 | 105 | 106 | codeine |
| EM3-0205 | 3.31 | 0.00 | 0.00 | 0.24 | 3.55 | 0.07 | 105 | 106 | codeine |
| EM3-0408 | 3.31 | 0.00 | 0.00 | 0.14 | 3.45 | 0.04 | 105 | 103 | codeine, T/C |
| EM3-1195 | 3.30 | 0.00 | 0.03 | 0.49 | 3.82 | 0.15 | 105 | 114 | codeine |
| EM3-0361 | 3.30 | 0.00 | 0.00 | 0.14 | 3.44 | 0.04 | 105 | 102 | codeine, T/C |
| EM3-1011 | 3.30 | 0.00 | 0.00 | 0.38 | 3.67 | 0.12 | 105 | 109 | codeine |
| EM3-0307 | 3.29 | 0.00 | 0.00 | 0.31 | 3.60 | 0.09 | 105 | 107 | codeine |
| EM3-1112 | 3.29 | 0.00 | 0.00 | 0.30 | 3.58 | 0.09 | 104 | 107 | codeine |
| EM3-0585 | 3.29 | 0.00 | 0.00 | 0.13 | 3.42 | 0.04 | 104 | 102 | codeine, T/C |
| EM3-1125 | 3.28 | 0.00 | 0.00 | 0.39 | 3.67 | 0.12 | 104 | 109 | codeine |
| EM3-1171 | 3.28 | 0.00 | 0.00 | 0.16 | 3.44 | 0.05 | 104 | 102 | codeine, T/C |
| EM3-0618 | 3.27 | 0.12 | 0.00 | 0.46 | 3.85 | 0.14 | 104 | 115 | codeine |
| EM3-0111 | 3.27 | 0.00 | 0.00 | 0.18 | 3.45 | 0.06 | 104 | 103 | codeine |
| EM3-0426 | 3.27 | 0.00 | 0.00 | 0.23 | 3.49 | 0.07 | 104 | 104 | codeine |
| EM3-1020 | 3.26 | 0.00 | 0.00 | 0.15 | 3.42 | 0.05 | 104 | 102 | codeine, T/C |
| EM3-1122 | 3.26 | 0.00 | 0.00 | 0.12 | 3.38 | 0.04 | 104 | 101 | codeine, T/C |
| EM3-1179 | 3.26 | 0.00 | 0.00 | 0.15 | 3.40 | 0.05 | 103 | 101 | codeine, T/C |
| EM3-1136 | 3.25 | 0.00 | 0.00 | 0.14 | 3.40 | 0.04 | 103 | 101 | codeine, T/C |
| EM3-0519 | 3.25 | 0.00 | 0.00 | 0.10 | 3.35 | 0.03 | 103 | 100 | codeine, T/C |
| EM3-0637 | 3.25 | 0.00 | 0.00 | 0.50 | 3.75 | 0.16 | 103 | 112 | codeine |
| EM3-0352 | 3.24 | 0.00 | 0.00 | 0.34 | 3.58 | 0.10 | 103 | 107 | codeine |
| EM3-0438 | 3.24 | 0.00 | 0.00 | 0.19 | 3.42 | 0.06 | 103 | 102 | codeine |
| EM3-0058 | 3.24 | 0.00 | 0.00 | 0.27 | 3.50 | 0.08 | 103 | 104 | codeine |
| EM3-0532 | 3.24 | 0.00 | 0.00 | 0.27 | 3.50 | 0.08 | 103 | 104 | codeine |
| EM3-0733 | 3.23 | 0.00 | 0.00 | 0.52 | 3.76 | 0.16 | 103 | 112 | codeine |
| EM3-0449 | 3.23 | 0.00 | 0.00 | 0.44 | 3.66 | 0.14 | 102 | 109 | codeine |
| EM3-0320 | 3.22 | 0.00 | 0.00 | 0.40 | 3.62 | 0.12 | 102 | 108 | codeine |
| EM3-0943 | 3.22 | 0.00 | 0.00 | 0.26 | 3.47 | 0.08 | 102 | 103 | codeine |
| EM3-0826 | 3.21 | 0.00 | 0.00 | 0.22 | 3.43 | 0.07 | 102 | 102 | codeine |
| EM3-1132 | 3.21 | 0.00 | 0.00 | 0.28 | 3.49 | 0.09 | 102 | 104 | codeine |
| EM3-0350 | 3.21 | 0.00 | 0.00 | 0.27 | 3.48 | 0.08 | 102 | 103 | codeine |
| EM3-0592 | 3.21 | 0.00 | 0.00 | 0.19 | 3.40 | 0.06 | 102 | 101 | codeine |
| EM3-1164 | 3.21 | 0.00 | 0.02 | 0.42 | 3.64 | 0.13 | 102 | 108 | codeine |
| EM3-0607 | 3.21 | 0.00 | 0.00 | 0.26 | 3.47 | 0.08 | 102 | 103 | codeine |
| EM3-1191 | 3.20 | 0.00 | 0.00 | 0.51 | 3.71 | 0.16 | 102 | 111 | codeine |
| EM3-0587 | 3.20 | 0.00 | 0.00 | 0.71 | 3.91 | 0.22 | 102 | 116 | codeine |
| EM3-1007 | 3.19 | 0.00 | 0.00 | 0.31 | 3.50 | 0.10 | 101 | 104 | codeine |
| EM3-0536 | 3.19 | 0.00 | 0.00 | 0.33 | 3.53 | 0.10 | 101 | 105 | codeine |
| EM3-0455 | 3.19 | 0.00 | 0.00 | 0.34 | 3.53 | 0.11 | 101 | 105 | codeine |
| EM3-0241 | 3.19 | 0.00 | 0.00 | 0.27 | 3.46 | 0.09 | 101 | 103 | codeine |
| EM3-0411 | 3.19 | 0.00 | 0.00 | 0.47 | 3.65 | 0.15 | 101 | 109 | codeine |
| EM3-0392 | 3.18 | 0.00 | 0.00 | 0.22 | 3.39 | 0.07 | 101 | 101 | codeine |
| EM3-1131 | 3.18 | 0.00 | 0.00 | 0.21 | 3.39 | 0.07 | 101 | 101 | codeine |
| EM3-0139 | 3.18 | 0.00 | 0.00 | 0.33 | 3.50 | 0.10 | 101 | 104 | codeine |
| EM3-0311 | 3.18 | 0.00 | 0.00 | 0.26 | 3.44 | 0.08 | 101 | 102 | codeine |
| EM3-0084 | 3.18 | 0.00 | 0.00 | 0.21 | 3.38 | 0.07 | 101 | 101 | codeine |
| EM3-1079 | 3.17 | 0.00 | 0.00 | 0.22 | 3.39 | 0.07 | 101 | 101 | codeine |
| EM3-0158 | 3.17 | 0.00 | 0.00 | 0.25 | 3.42 | 0.08 | 101 | 102 | codeine |
| EM3-0200 | 3.17 | 0.09 | 0.00 | 0.34 | 3.60 | 0.11 | 101 | 107 | codeine |
| EM3-1163 | 3.17 | 0.00 | 0.00 | 0.33 | 3.50 | 0.10 | 101 | 104 | codeine |
| EM3-0646 | 3.16 | 0.00 | 0.00 | 0.16 | 3.32 | 0.05 | 100 | 99 | T/C |
| EM3-1161 | 3.15 | 0.00 | 0.00 | 0.16 | 3.31 | 0.05 | 100 | 98 | T/C |
| EM3-0484 | 3.12 | 0.00 | 0.00 | 0.17 | 3.28 | 0.05 | 99 | 98 | T/C |
| EM3-1039 | 3.12 | 0.00 | 0.00 | 0.08 | 3.20 | 0.03 | 99 | 95 | T/C |
| EM3-0698 | 3.09 | 0.00 | 0.00 | 0.15 | 3.24 | 0.05 | 98 | 96 | T/C |
| EM3-1113 | 3.09 | 0.00 | 0.00 | 0.17 | 3.25 | 0.05 | 98 | 97 | T/C |
| EM3-1116 | 3.07 | 0.00 | 0.00 | 0.12 | 3.20 | 0.04 | 98 | 95 | T/C |
| EM3-1159 | 3.07 | 0.00 | 0.00 | 0.15 | 3.22 | 0.05 | 98 | 96 | T/C |
| EM3-0151 | 3.07 | 0.00 | 0.00 | 0.08 | 3.15 | 0.03 | 98 | 94 | T/C |
| EM3-1022 | 3.07 | 0.00 | 0.00 | 0.13 | 3.20 | 0.04 | 97 | 95 | T/C |
| EM3-1110 | 3.05 | 0.00 | 0.00 | 0.06 | 3.10 | 0.02 | 97 | 92 | T/C |
| EM3-1183 | 3.05 | 0.00 | 0.00 | 0.08 | 3.13 | 0.02 | 97 | 93 | T/C |
| EM3-0501 | 3.04 | 0.00 | 0.00 | 0.10 | 3.14 | 0.03 | 96 | 93 | T/C |
| EM3-0273 | 3.02 | 0.00 | 0.00 | 0.07 | 3.09 | 0.02 | 96 | 92 | T/C |
| EM3-0498 | 3.02 | 0.00 | 0.00 | 0.10 | 3.12 | 0.03 | 96 | 93 | T/C |
| EM3-0843 | 3.01 | 0.00 | 0.00 | 0.06 | 3.07 | 0.02 | 96 | 91 | T/C |
| EM3-1066 | 3.01 | 0.00 | 0.01 | 0.16 | 3.18 | 0.05 | 96 | 95 | T/C |
| EM3-0183 | 3.01 | 0.00 | 0.00 | 0.08 | 3.09 | 0.03 | 96 | 92 | T/C |
| EM3-0664 | 3.01 | 0.00 | 0.00 | 0.14 | 3.14 | 0.05 | 95 | 94 | T/C |
| EM3-0617 | 3.00 | 0.00 | 0.00 | 0.11 | 3.11 | 0.04 | 95 | 93 | T/C |
| EM3-0208 | 2.99 | 0.00 | 0.00 | 0.12 | 3.11 | 0.04 | 95 | 93 | T/C |
| EM3-0284 | 2.98 | 0.00 | 0.00 | 0.14 | 3.12 | 0.05 | 95 | 93 | T/C |
| EM3-0071 | 2.98 | 0.00 | 0.00 | 0.16 | 3.14 | 0.05 | 95 | 94 | T/C |
| EM3-0653 | 2.98 | 0.00 | 0.00 | 0.16 | 3.14 | 0.05 | 95 | 93 | T/C |
| EM3-0577 | 2.98 | 0.00 | 0.00 | 0.16 | 3.13 | 0.05 | 94 | 93 | T/C |
| EM3-1117 | 2.96 | 0.00 | 0.00 | 0.16 | 3.13 | 0.05 | 94 | 93 | T/C |
| EM3-0573 | 2.96 | 0.00 | 0.00 | 0.12 | 3.08 | 0.04 | 94 | 92 | T/C |
| EM3-0730 | 2.96 | 0.00 | 0.06 | 0.16 | 3.17 | 0.05 | 94 | 94 | T/C |
| EM3-1184 | 2.96 | 0.00 | 0.00 | 0.12 | 3.07 | 0.04 | 94 | 91 | T/C |
| EM3-0515 | 2.95 | 0.00 | 0.00 | 0.04 | 3.00 | 0.01 | 94 | 89 | T/C |
| EM3-0638 | 2.95 | 0.09 | 0.00 | 0.08 | 3.11 | 0.03 | 94 | 93 | T/C |
| EM3-0990 | 2.94 | 0.00 | 0.00 | 0.06 | 3.00 | 0.02 | 93 | 89 | T/C |
| EM3-0301 | 2.94 | 0.00 | 0.00 | 0.15 | 3.09 | 0.05 | 93 | 92 | T/C |
| EM3-0333 | 2.94 | 0.00 | 0.00 | 0.13 | 3.07 | 0.05 | 93 | 91 | T/C |
| EM3-0132 | 2.93 | 0.00 | 0.00 | 0.14 | 3.07 | 0.05 | 93 | 91 | T/C |
| EM3-0467 | 2.93 | 0.00 | 0.00 | 0.10 | 3.03 | 0.03 | 93 | 90 | T/C |
| EM3-0424 | 2.92 | 0.00 | 0.00 | 0.08 | 2.99 | 0.03 | 93 | 89 | T/C |
| EM3-0611 | 2.92 | 0.00 | 0.00 | 0.09 | 3.00 | 0.03 | 93 | 89 | T/C |
| EM3-0633 | 2.91 | 0.00 | 0.00 | 0.14 | 3.05 | 0.05 | 93 | 91 | T/C |
| EM3-1069 | 2.91 | 0.00 | 0.00 | 0.15 | 3.06 | 0.05 | 92 | 91 | T/C |
| EM3-0477 | 2.91 | 0.00 | 0.00 | 0.15 | 3.06 | 0.05 | 92 | 91 | T/C |
| EM3-0094 | 2.91 | 0.00 | 0.00 | 0.14 | 3.05 | 0.05 | 92 | 91 | T/C |
| EM3-1162 | 2.91 | 0.00 | 0.00 | 0.12 | 3.02 | 0.04 | 92 | 90 | T/C |
| EM3-0635 | 2.91 | 0.00 | 0.00 | 0.12 | 3.02 | 0.04 | 92 | 90 | T/C |
| EM3-0898 | 2.90 | 0.00 | 0.00 | 0.15 | 3.05 | 0.05 | 92 | 91 | T/C |
| EM3-0170 | 2.90 | 0.00 | 0.00 | 0.13 | 3.02 | 0.04 | 92 | 90 | T/C |
| EM3-0737 | 2.90 | 0.00 | 0.00 | 0.16 | 3.05 | 0.05 | 92 | 91 | T/C |
| EM3-0414 | 2.90 | 0.00 | 0.01 | 0.16 | 3.06 | 0.05 | 92 | 91 | T/C |
| EM3-0555 | 2.89 | 0.00 | 0.00 | 0.13 | 3.02 | 0.04 | 92 | 90 | T/C |
| EM3-0871 | 2.89 | 0.00 | 0.00 | 0.16 | 3.05 | 0.05 | 92 | 91 | T/C |
| EM3-1009 | 2.89 | 0.00 | 0.00 | 0.11 | 2.99 | 0.04 | 92 | 89 | T/C |
| EM3-0868 | 2.88 | 0.00 | 0.01 | 0.14 | 3.04 | 0.05 | 92 | 90 | T/C |
| EM3-0928 | 2.88 | 0.00 | 0.00 | 0.13 | 3.01 | 0.04 | 91 | 90 | T/C |
| EM3-0558 | 2.87 | 0.00 | 0.00 | 0.15 | 3.02 | 0.05 | 91 | 90 | T/C |
| EM3-0383 | 2.87 | 0.00 | 0.00 | 0.07 | 2.93 | 0.02 | 91 | 87 | T/C |
| EM3-0726 | 2.86 | 0.00 | 0.00 | 0.10 | 2.96 | 0.03 | 91 | 88 | T/C |
| EM3-0832 | 2.86 | 0.00 | 0.00 | 0.04 | 2.89 | 0.01 | 91 | 86 | T/C |
| EM3-0670 | 2.85 | 0.00 | 0.00 | 0.13 | 2.99 | 0.05 | 91 | 89 | T/C |
| EM3-0434 | 2.85 | 0.00 | 0.00 | 0.14 | 2.99 | 0.05 | 91 | 89 | T/C |
| EM3-0066 | 2.85 | 0.00 | 0.00 | 0.08 | 2.92 | 0.03 | 90 | 87 | T/C |
| EM3-0925 | 2.85 | 0.00 | 0.00 | 0.10 | 2.95 | 0.03 | 90 | 88 | T/C |
| EM3-1182 | 2.85 | 0.00 | 0.00 | 0.13 | 2.98 | 0.05 | 90 | 89 | T/C |
| EM3-1032 | 2.85 | 0.00 | 0.00 | 0.06 | 2.91 | 0.02 | 90 | 87 | T/C |
| EM3-1108 | 2.85 | 0.00 | 0.00 | 0.11 | 2.95 | 0.04 | 90 | 88 | T/C |
| EM3-1076 | 2.84 | 0.00 | 0.00 | 0.16 | 2.99 | 0.05 | 90 | 89 | T/C |
| EM3-0496 | 2.84 | 0.00 | 0.00 | 0.09 | 2.93 | 0.03 | 90 | 87 | T/C |
| EM3-0181 | 2.83 | 0.00 | 0.00 | 0.12 | 2.95 | 0.04 | 90 | 88 | T/C |
| EM3-1002 | 2.82 | 0.00 | 0.00 | 0.13 | 2.95 | 0.04 | 90 | 88 | T/C |
| EM3-0428 | 2.82 | 0.00 | 0.00 | 0.12 | 2.93 | 0.04 | 89 | 87 | T/C |
| EM3-0304 | 2.81 | 0.00 | 0.00 | 0.14 | 2.95 | 0.05 | 89 | 88 | T/C |
| EM3-1148 | 2.81 | 0.00 | 0.00 | 0.09 | 2.90 | 0.03 | 89 | 86 | T/C |
| EM3-0480 | 2.81 | 0.00 | 0.00 | 0.10 | 2.90 | 0.03 | 89 | 86 | T/C |
| EM3-0533 | 2.80 | 0.12 | 0.00 | 0.14 | 3.06 | 0.05 | 89 | 91 | T/C |
| EM3-0365 | 2.79 | 0.00 | 0.00 | 0.05 | 2.84 | 0.02 | 89 | 85 | T/C |
| EM3-0238 | 2.78 | 0.00 | 0.00 | 0.12 | 2.91 | 0.04 | 88 | 86 | T/C |
| EM3-1197 | 2.78 | 0.00 | 0.00 | 0.09 | 2.87 | 0.03 | 88 | 85 | T/C |
| EM3-0435 | 2.78 | 0.00 | 0.00 | 0.12 | 2.90 | 0.04 | 88 | 86 | T/C |
| EM3-0788 | 2.77 | 0.00 | 0.00 | 0.09 | 2.85 | 0.03 | 88 | 85 | T/C |
| EM3-0731 | 2.76 | 0.00 | 0.00 | 0.14 | 2.91 | 0.05 | 88 | 86 | T/C |
| EM3-0984 | 2.76 | 0.00 | 0.00 | 0.08 | 2.84 | 0.03 | 88 | 84 | T/C |
| EM3-1001 | 2.74 | 0.00 | 0.00 | 0.10 | 2.85 | 0.04 | 87 | 85 | T/C |
| EM3-0729 | 2.73 | 0.00 | 0.00 | 0.13 | 2.87 | 0.05 | 87 | 85 | T/C |
| EM3-0549 | 2.73 | 0.00 | 0.00 | 0.11 | 2.84 | 0.04 | 87 | 84 | T/C |
| EM3-0371 | 2.73 | 0.00 | 0.00 | 0.13 | 2.86 | 0.05 | 87 | 85 | T/C |
| EM3-0791 | 2.72 | 0.00 | 0.00 | 0.09 | 2.81 | 0.03 | 86 | 84 | T/C |
| EM3-0739 | 2.71 | 0.00 | 0.00 | 0.10 | 2.81 | 0.04 | 86 | 84 | T/C |
| EM3-0283 | 2.70 | 0.00 | 0.00 | 0.14 | 2.84 | 0.05 | 86 | 85 | T/C |
| EM3-1114 | 2.70 | 0.00 | 0.00 | 0.14 | 2.84 | 0.05 | 86 | 85 | T/C |
| EM3-0651 | 2.70 | 0.00 | 0.00 | 0.12 | 2.82 | 0.04 | 86 | 84 | T/C |
| EM3-0566 | 2.70 | 0.00 | 0.00 | 0.12 | 2.81 | 0.04 | 86 | 84 | T/C |
| EM3-0131 | 2.69 | 0.00 | 0.00 | 0.15 | 2.84 | 0.05 | 86 | 85 | T/C |
| EM3-0358 | 2.68 | 0.00 | 0.00 | 0.06 | 2.74 | 0.02 | 85 | 82 | T/C |
| EM3-0382 | 2.68 | 0.00 | 0.00 | 0.05 | 2.73 | 0.02 | 85 | 81 | T/C |
| EM3-1151 | 2.67 | 0.00 | 0.00 | 0.13 | 2.81 | 0.05 | 85 | 84 | T/C |
| EM3-0275 | 2.67 | 0.00 | 0.00 | 0.10 | 2.78 | 0.04 | 85 | 83 | T/C |
| EM3-0713 | 2.66 | 0.00 | 0.00 | 0.10 | 2.76 | 0.04 | 84 | 82 | T/C |
| EM3-1036 | 2.66 | 0.00 | 0.00 | 0.05 | 2.71 | 0.02 | 84 | 81 | T/C |
| EM3-0728 | 2.65 | 0.00 | 0.00 | 0.11 | 2.77 | 0.04 | 84 | 82 | T/C |
| EM3-0834 | 2.65 | 0.00 | 0.00 | 0.13 | 2.78 | 0.05 | 84 | 83 | T/C |
| EM3-0281 | 2.64 | 0.00 | 0.00 | 0.02 | 2.67 | 0.01 | 84 | 79 | T/C |
| EM3-0376 | 2.64 | 0.00 | 0.00 | 0.09 | 2.73 | 0.04 | 84 | 81 | T/C |
| EM3-0615 | 2.64 | 0.00 | 0.00 | 0.14 | 2.78 | 0.05 | 84 | 83 | T/C |
| EM3-0528 | 2.63 | 0.00 | 0.00 | 0.12 | 2.75 | 0.05 | 84 | 82 | T/C |
| EM3-0305 | 2.63 | 0.00 | 0.00 | 0.10 | 2.73 | 0.04 | 84 | 81 | T/C |
| EM3-0322 | 2.61 | 0.00 | 0.00 | 0.12 | 2.74 | 0.05 | 83 | 81 | T/C |
| EM3-0560 | 2.60 | 0.09 | 0.00 | 0.12 | 2.81 | 0.05 | 83 | 84 | T/C |
| EM3-0492 | 2.60 | 0.00 | 0.00 | 0.10 | 2.70 | 0.04 | 82 | 80 | T/C |
| EM3-0293 | 2.59 | 0.00 | 0.00 | 0.11 | 2.70 | 0.04 | 82 | 80 | T/C |
| EM3-0794 | 2.58 | 0.00 | 0.00 | 0.08 | 2.67 | 0.03 | 82 | 79 | T/C |
| EM3-0894 | 2.57 | 0.00 | 0.00 | 0.10 | 2.66 | 0.04 | 81 | 79 | T/C |
| EM3-0608 | 2.56 | 0.17 | 0.00 | 0.12 | 2.85 | 0.05 | 81 | 85 | T/C |
| EM3-0767 | 2.55 | 0.00 | 0.00 | 0.10 | 2.65 | 0.04 | 81 | 79 | T/C |
| EM3-1118 | 2.55 | 0.00 | 0.00 | 0.13 | 2.68 | 0.05 | 81 | 80 | T/C |
| EM3-0538 | 2.55 | 0.00 | 0.00 | 0.02 | 2.57 | 0.01 | 81 | 76 | T/C |
| EM3-0892 | 2.53 | 0.00 | 0.00 | 0.13 | 2.65 | 0.05 | 80 | 79 | T/C |
| EM3-0857 | 2.49 | 0.09 | 0.00 | 0.13 | 2.71 | 0.05 | 79 | 81 | T/C |
| EM3-0246 | 2.48 | 0.00 | 0.00 | 0.07 | 2.56 | 0.03 | 79 | 76 | T/C |
| EM3-0389 | 2.42 | 0.10 | 0.00 | 0.05 | 2.57 | 0.02 | 77 | 76 | T/C |
| EM3-0298 | 2.36 | 0.00 | 0.00 | 0.06 | 2.42 | 0.03 | 75 | 72 | T/C |
| EM3-0825 | 2.32 | 0.00 | 0.00 | 0.09 | 2.42 | 0.04 | 74 | 72 | T/C |
| EM3-0297 | 2.30 | 0.00 | 0.00 | 0.10 | 2.40 | 0.04 | 73 | 71 | T/C |
| EM3-0644 | 2.25 | 0.00 | 0.00 | 0.07 | 2.31 | 0.03 | 71 | 69 | T/C |
| EM3-1075 | 2.12 | 0.00 | 0.00 | 0.07 | 2.19 | 0.03 | 67 | 65 | T/C |

MOCT is the alkaloid combination morphine, oripavine, codeine and thebaine.

### 2. M3 Disease Resistance/Phenotype Screening Trial

A disease resistance screening field trial (Hagley, Tasmania, Australia) was also conducted during the 2012/13 poppy growing season. A subset of 11 mutant lines identified on the basis of phenotype in the M2 (field) and M3 (planthouse) generations described above were selected for inclusion in this trial, including the ‘Tasman’ line EM4-0045 derived from parent line PW08-2308 by mutagenesis treatment with EMS as described in Example 1. The primary aim of this trial was to determine if the altered phenotypes resulted in any improvement in resistance to, or tolerance of, downy mildew (Peronospora meconosidis, previously known as Peronospora arborescens) infection. This trial was also an additional opportunity to further study plant phenotypes under field conditions. M4 seed, bulked from multiple M3 plants of each line grown in the planthouse over the winter of 2013, was used to sow this trial. The trial was a randomized-complete-block-design with 3 blocks/replicates per line, each replicate was sown a 1.8 m wide×2.0 m long plot. The trial crop was not sprayed with any preventative or curative fungicides. Plants were allowed to mature and dry under field conditions, and 30 capsules were then harvested from each plot and combined for each individual replicate assay. Each 30-capsule-sample was threshed to remove seed to produce poppy straw, the straw was then weighed and ground, and the ground straw was extracted in 2% acetic acid and 10% ethanol in distilled water as described above. An aliquot of the extract was filtered prior to the UPLC analysis of alkaloid content and alkaloid profile as also described above. Data derived from the three replicate plots was analysed in Agrobase Generation II (Agronomix Software) using GLM (General Linear Model) analysis to produce an overall trial mean for each alkaloid in each M3 line.

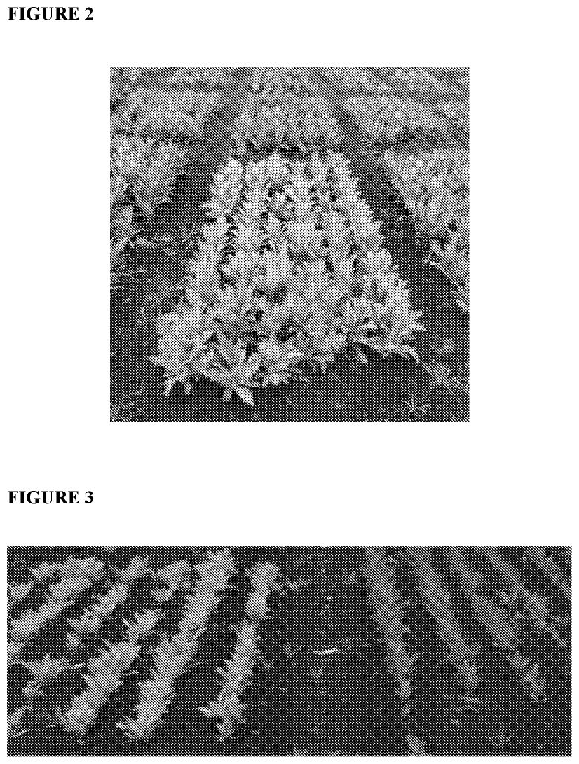

There was no expectation that altered phenotypes of the mutant lines would result in an increase in alkaloid content, so the actual mutagenesis parent line (PW08-2308) was not been entered in the trial for comparison. However, ‘Tasman’ mutant line EM4-0045 surprisingly exhibited high-total alkaloid content and an altered leaf phenotype characterized by light green-yellow leaves (see FIG. 2).

In a subsequent field trial (data not shown), the EM4-0045 light green-yellow ‘Tasman’ mutant line exhibited a 5% increase in codeine relative to the parent line PW08-2308. Apart from the light green-yellow leaf colour, the phenotype of this line appears to be unchanged relative to the parent line, PW08-2308. As such, there appears to be no reason why the EM4-0045 mutant cannot be grown commercially.

### 3. Discussion

Various M2 plants were selected on the basis of altered phenotypes as outlined in Example 1. Some of these phenotypes were also present when M3 plants, derived from these M2 plants, were grown in various M3 screening trials. This demonstrates that these phenotypes are the result of stable, heritable, genetic changes, as the altered phenotypes were consistent across generations and environments.

An unexpected result from these trials was the identification of mutant lines that exhibited lighter coloured leaves (i.e., lighter green-yellow or very light green-yellow) and which contained high alkaloid content, suggesting a possible relationship between the altered leaf colour in these lines and alkaloid accumulation. These plants were not initially considered as being of interest in the M2 screen, and were harvested simply as a curiosity. Growing M3 seed from these lines in the greenhouse highlighted that these mutant phenotypes were both stable and heritable. Mutant line EM4-0045 in particular showed remarkably good vigour and its phenotype was largely unchanged relative to the parent line PW08-2308 other than the obvious difference in leaf colour. This line was included in the 2012/2013 disease resistance screening field trial to assess whether their phenotypic differences altered their level of resistance to downy mildew infection (Peronospora meconosidis). Whilst this proved to not to be the case and no apparent enhancement in protection against downy mildew infection was observed, the EM4-0045 (light green-yellow leaf) ‘Tasman’ mutant exhibited very high codeine content with low thebaine contamination, a most striking and unexpected result. As a consequence, the EM4-0045 line was advanced to additional field trials for further assessment.

Overall, M3 Papaver somniferum mutant ‘Tasman’ lines were identified which showed an increase in codeine content relative to the parent line from which they were derived, as well as mutant ‘Tasman’ lines which also exhibited a reduction in thebaine content relative to codeine content. Given the nature of mutagenesis, the complex nature of alkaloid biosynthesis pathways in Papaver somniferum and the involvement of feedback mechanisms and the like as described above, it could not be predicted at the outset if any desirable changes in alkaloid content or profile were possible or if they could even be achieved via mutagenesis, which typically results in point mutations that either knock out or have a detrimental effect on gene function. Further, it could not be ascertained or predicted in advance as to whether any plants having altered phenotypes could be useful commercially.

### EXAMPLE 3

### Breeding Material Field Trial (Hagley, Tasmania, Australia)

A total of 82 mutant ‘Tasman’ M3 lines produced in Example 1 and their parent line, PW08-2308, were included in this field trial. The trial was conducted in a paddock (Hagley, Tasmania, Australia) during the 2013/14 poppy growing season, and was sown on 6 Sep. 2013. The trial was a randomized-complete-block design consisting of 2 blocks/replicates of each M3 line. Each replicate was sown in a single, 5 m long row within a 5 m×1.8 m plot that contained five individual rows. A visual assessment of plant phenotype was conducted prior to flowering. Plants were allowed to mature and dry under field conditions, and all of the capsules from plants in each row were then harvested (on 5 Feb. 2014) and pooled for analysis of seed and straw weight, and alkaloid content and profile. Capsules were weighed and threshed to remove seed to produce straw. The straw was weighed and ground, and the ground straw was extracted in 2% acetic acid and 10% ethanol in distilled water as described above. An aliquot of the extract was filtered prior to UPLC analysis of alkaloid content and alkaloid profile as also described above. The results for each M3 line were analysed in Agrobase Generation II (Agronomix Software) using GLM (General Linear Model) analysis and compared to the parent line, PW08-2308, and are set out below in Table 2.

TABLE 2
Alkaloid content of poppy straw of selected P. somniferum M3 lines

| | Codeine | Thebaine | % C of | Total | | Codeine |
|---|---|---|---|---|---|---|
| | (C) | (T) | PW08- | T + C | T/C | yield |
| Line | (% w/w) | (% w/w) | 2308 | (% w/w) | ratio | (kg/Ha) |
| PW08-2308 | 3.5 | 0.4 | 100 | 3.9 | 0.11 | 29.0 |
| EM3-0352 | 4.2 | 0.5 | 120 | 4.6 | 0.11 | 43.9 |
| EM3-0006 | 4.1 | 0.1 | 119 | 4.2 | 0.02 | 49.8 |
| EM3-0106 | 4.1 | 0.3 | 118 | 4.4 | 0.07 | 34.5 |
| EM3-0587 | 4.1 | 0.4 | 116 | 4.5 | 0.11 | 39.4 |
| EM3-0455 | 4.0 | 0.3 | 116 | 4.3 | 0.07 | 39.8 |
| EM3-1026 | 4.0 | 0.2 | 116 | 4.2 | 0.04 | 70.6 |
| EM3-0426 | 4.0 | 0.1 | 116 | 4.2 | 0.04 | 26.3 |
| EM3-1123 | 4.0 | 0.3 | 115 | 4.3 | 0.07 | 41.3 |
| EM3-0505 | 4.0 | 0.4 | 115 | 4.4 | 0.09 | 58.2 |
| EM3-0350 | 4.0 | 0.3 | 114 | 4.3 | 0.07 | 60.3 |
| EM3-0766 | 3.9 | 0.5 | 113 | 4.4 | 0.13 | 21.7 |
| EM3-1125 | 3.9 | 0.3 | 113 | 4.2 | 0.04 | 37.3 |
| EM3-1136 | 3.9 | 0.2 | 112 | 4.1 | 0.06 | 46.9 |
| EM3-0479 | 3.9 | 0.2 | 112 | 4.1 | 0.05 | 56.7 |
| EM3-1164 | 3.9 | 0.2 | 112 | 4.1 | 0.05 | 49.4 |
| EM3-1179 | 3.9 | 0.1 | 112 | 4.1 | 0.02 | 39.0 |
| EM3-0733 | 3.9 | 0.3 | 111 | 4.2 | 0.08 | 69.6 |
| EM3-0408 | 3.9 | 0.1 | 111 | 4.0 | 0.04 | 47.3 |
| EM3-1045 | 3.9 | 0.8 | 111 | 4.6 | 0.20 | 34.2 |
| EM3-0407 | 3.8 | 0.3 | 110 | 4.2 | 0.08 | 49.6 |
| EM3-0686 | 3.8 | 0.3 | 109 | 4.2 | 0.09 | 61.5 |
| EM3-1124 | 3.8 | 0.1 | 109 | 3.9 | 0.04 | 2.9 |
| EM3-1138 | 3.8 | 0.1 | 109 | 3.9 | 0.04 | 17.3 |
| EM3-0056 | 3.8 | 0.2 | 108 | 4.1 | 0.06 | 30.3 |
| EM3-0826 | 3.8 | 0.2 | 108 | 4.0 | 0.05 | 43.2 |
| EM3-0285 | 3.8 | 0.2 | 108 | 4.0 | 0.07 | 58.4 |
| EM3-0331 | 3.8 | 0.4 | 108 | 4.3 | 0.10 | 57.3 |
| EM3-0023 | 3.8 | 0.2 | 108 | 4.1 | 0.06 | 51.2 |
| EM3-0411 | 3.8 | 0.4 | 108 | 4.1 | 0.10 | 41.3 |
| EM3-0191 | 3.7 | 0.3 | 107 | 4.3 | 0.05 | 29.0 |
| EM3-1196 | 3.7 | 0.3 | 107 | 4.1 | 0.09 | 21.5 |
| EM3-0592 | 3.7 | 0.4 | 107 | 4.1 | 0.10 | 46.3 |
| EM3-0072 | 3.7 | 0.3 | 107 | 4.1 | 0.08 | 45.1 |
| EM3-0111 | 3.7 | 0.3 | 107 | 4.0 | 0.07 | 28.2 |
| EM3-1122 | 3.7 | 0.2 | 107 | 3.9 | 0.05 | 30.1 |
| EM3-0018 | 3.7 | 0.3 | 107 | 4.0 | 0.04 | 45.0 |
| EM3-1112 | 3.7 | 0.4 | 106 | 4.1 | 0.12 | 25.1 |
| EM3-0448 | 3.7 | 0.5 | 106 | 4.3 | 0.13 | 35.0 |
| EM3-0607 | 3.7 | 0.3 | 106 | 4.0 | 0.09 | 72.0 |
| EM3-0674 | 3.7 | 0.4 | 106 | 4.1 | 0.10 | 43.2 |
| EM3-0637 | 3.7 | 0.2 | 106 | 3.9 | 0.05 | 24.7 |
| EM3-0076 | 3.7 | 0.4 | 105 | 4.1 | 0.11 | 40.6 |
| EM3-0519 | 3.7 | 0.3 | 105 | 4.0 | 0.08 | 64.2 |
| EM3-0476 | 3.7 | 0.1 | 105 | 3.8 | 0.02 | 47.9 |
| EM3-1011 | 3.7 | 0.4 | 105 | 4.1 | 0.11 | 57.7 |
| EM3-1007 | 3.7 | 0.2 | 105 | 3.9 | 0.06 | 22.0 |
| EM3-0619 | 3.6 | 0.3 | 104 | 4.0 | 0.09 | 42.4 |
| EM3-1132 | 3.6 | 0.4 | 104 | 4.1 | 0.12 | 37.6 |
| EM3-1166 | 3.6 | 0.3 | 103 | 3.9 | 0.09 | 16.2 |
| EM3-0585 | 3.6 | 0.4 | 103 | 4.0 | 0.11 | 22.0 |
| EM3-0390 | 3.6 | 0.2 | 103 | 3.8 | 0.06 | 45.3 |
| EM3-0241 | 3.6 | 0.3 | 103 | 4.0 | 0.09 | 53.4 |
| EM3-0584 | 3.6 | 0.3 | 103 | 3.9 | 0.09 | 34.0 |
| EM3-0058 | 3.6 | 0.5 | 102 | 4.1 | 0.08 | 60.6 |
| EM3-1020 | 3.5 | 0.2 | 102 | 3.8 | 0.07 | 46.8 |
| EM3-0552 | 3.5 | 0.3 | 101 | 4.0 | 0.09 | 30.0 |
| EM3-1180 | 3.5 | 0.2 | 101 | 3.9 | 0.07 | 37.3 |
| EM3-0539 | 3.5 | 0.2 | 101 | 3.8 | 0.07 | 23.7 |
| EM3-0764 | 3.5 | 0.9 | 101 | 4.4 | 0.13 | 58.7 |
| EM3-0034 | 3.5 | 0.5 | 101 | 4.0 | 0.16 | 36.0 |
| EM3-0551 | 3.5 | 0.4 | 101 | 3.9 | 0.06 | 47.7 |
| EM3-0307 | 3.5 | 0.2 | 100 | 3.8 | 0.06 | 34.0 |
| EM3-1131 | 3.5 | 0.3 | 100 | 3.7 | 0.07 | 71.7 |
| EM3-0205 | 3.5 | 0.3 | 100 | 3.8 | 0.08 | 39.4 |
| EM3-0801 | 3.5 | 0.5 | 99 | 4.0 | 0.14 | 32.9 |
| EM3-0361 | 3.4 | 0.2 | 99 | 3.9 | 0.04 | 58.4 |
| EM3-0506 | 3.4 | 0.4 | 99 | 3.9 | 0.13 | 49.0 |
| EM3-1147 | 3.4 | 0.4 | 98 | 3.8 | 0.06 | 46.5 |
| EM3-1198 | 3.4 | 0.9 | 97 | 4.3 | 0.27 | 30.4 |
| EM3-1171 | 3.3 | 0.4 | 95 | 3.7 | 0.11 | 28.5 |
| EM3-0095 | 3.3 | 0.5 | 94 | 3.9 | 0.14 | 27.7 |
| EM3-1199 | 3.3 | 0.8 | 94 | 4.1 | 0.28 | 50.1 |
| EM3-0320 | 3.3 | 0.7 | 94 | 4.0 | 0.23 | 43.9 |
| EM3-1191 | 3.2 | 0.5 | 92 | 3.9 | 0.19 | 27.2 |
| EM3-0449 | 3.2 | 0.7 | 91 | 3.8 | 0.22 | 23.3 |
| EM3-0532 | 3.1 | 0.4 | 90 | 3.6 | 0.12 | 21.5 |
| EM3-0934 | 3.1 | 0.4 | 88 | 3.7 | 0.14 | 33.5 |
| EM3-0643 | 2.9 | 0.5 | 84 | 3.8 | 0.19 | 21.0 |
| EM3-1195 | 2.9 | 0.3 | 84 | 3.8 | 0.11 | 25.1 |
| EM3-0438 | 2.8 | 0.2 | 81 | 3.8 | 0.06 | 25.8 |
| EM3-0618 | 2.8 | 0.6 | 81 | 3.6 | 0.10 | 38.7 |
| EM3-0536 | 2.5 | 0.6 | 71 | 3.1 | 0.32 | 30.7 |

Of the 82 lines tested, 61 showed improvement in codeine content relative to the PW08-2308 parent line. The best of these M3 lines, EM3-0352, exhibited a 20% improvement in codeine content. Interestingly, the next best M3 line, EM3-0006, which exhibited a 19% improvement in codeine content, also exhibited a very light green-yellow leaf phenotype. This result is consistent with the study described in Example 2 above which show high-alkaloid content in other M3 lines that exhibit a similar light leaf colour phenotype compared to the parent line PW08-2308. The co-occurrence of the lighter (light green-yellow and very light green-yellow) leaf colour traits in several independent mutant lines suggests mutations in genes that can either directly, or indirectly, influence leaf colour as well as alkaloid content and alkaloid profile.

In addition to those lines that exhibited increases in codeine content, 56 M3 lines also exhibited lower thebaine content relative to codeine (T/C), than PW08-2308, which exhibited a T/C value of 0.11. The best of these M3 lines were EM3-0006 (very light green-yellow leaf phenotype), EM3-1179 and EM3-0476, which all exhibited a T/C of 0.02. This suggests that, in the current trial location and season these lines were more efficient at converting thebaine through to codeine. When coupled with a high codeine content this low T/C trait is particularly valuable as it maximizes the amount of codeine available for extraction whilst reducing the level of thebaine that needs to be removed during factory processing.

### EXAMPLE 4

### Multi-Location Trials

Six field trials were sown in the 2014/15 poppy growing season with the purpose of identifying mutant Tasman lines with wide adaptability to different growing regions. The trials contained 18 entries including 3 commercially grown high codeine ‘Tasman’ Papaver somniferum producing lines, namely PW08-2308, PW11-4027 and PW11-4118. The trials were sown across a wide geographical distribution (Tunbridge, Cressy, Perth, Hagley, Latrobe, and North Motton), representing a diverse range of poppy growing regions in Tasmania, Australia. Each trial was sown within a poppy paddock and was treated the same as the surrounding crop in regards to herbicide, fertilizer, fungicide and irrigation treatments. Trial plants were not sprayed with any plant growth regulators.

Plants were allowed to mature and dry under field conditions. Immediately prior to the harvest of the surrounding commercial crop, all capsules from within a designated quadrat were hand-harvested from each trial plot. The harvested straw samples therefore consisted only of plant capsules and seed and contained no plant stems. All harvested material was stored for up to one month before being threshed to remove seed. These straw samples were ground, and the ground straw was extracted in 2% acetic acid and 10% ethanol in distilled water as described above. An aliquot of the extract was filtered prior to UPLC analysis of alkaloid content and alkaloid profile as also described above.

For each individual trial site, the alkaloid (% w/w) results for the three replicates of each line were analysed in Agrobase Generation II (Agronomix Software) using “Alpha” analysis to produce a mean value for that line at that trial site. An overall trial mean for each alkaloid (% w/w), from across the 6 trial sites, was then determined in Agrobase Generation II using the GxE Analysis function “ANOVA-combined RCBD: ENV. X ENTRY model. These overall trial means for codeine and thebaine content and related traits for these trials are shown in Table 3.

TABLE 3
Codeine and thebaine content of poppy straw from Papaver somniferum lines in 2014/2015 field trials

| | | | Total | | | | | Codeine |
|---|---|---|---|---|---|---|---|---|
| | Codeine | Thebaine | MOCT | Thebaine/ | Codeine % | Thebaine % | Total % | yield % |
| Line | (% w/w) | (% w/w) | (% w/w) | codeine | of PW08-2308 | of PW08-2308 | of PW08-2308 | of PW08-2308 |
| PW08-2308 | 3.50 | 0.35 | 3.89 | 0.09 | 100 | 100 | 100 | 100 |
| PW11-4027 | 3.52 | 0.29 | 3.85 | 0.09 | 101 | 83 | 99 | 96 |
| PW11-4118 | 3.57 | 0.29 | 3.90 | 0.10 | 102 | 81 | 100 | 94 |
| EM4-0045 | 4.01 | 0.20 | 4.21 | 0.06 | 115 | 56 | 108 | 103 |
| EM3-1204 | 3.88 | 0.21 | 4.07 | 0.06 | 111 | 61 | 105 | 102 |
| EM3-1217 | 3.83 | 0.31 | 4.17 | 0.09 | 109 | 90 | 107 | 103 |
| EM3-0056 | 3.73 | 0.41 | 4.16 | 0.12 | 107 | 116 | 107 | 93 |
| EM4-0270 | 3.64 | 0.30 | 3.95 | 0.08 | 104 | 85 | 102 | 89 |
| EM3-1203 | 3.44 | 0.56 | 4.02 | 0.18 | 98 | 159 | 103 | 93 |
| EM3-1213 | 3.39 | 0.33 | 3.77 | 0.07 | 97 | 94 | 97 | 78 |

Of the 7 mutant ‘Tasman’ lines evaluated in the present trials, 5 of the lines showed an improvement in codeine content (relative to the predominant commercial variety, PW08-2308) of between 4 and 15% on a % w/w basis. The best of these new lines was the light green-yellow leaf mutant line EM4-0045, which exhibited the highest codeine content at 4 of the 6 trial sites.

Mutant lines EM3-1204 and EM3-1217 both showed high vigour and exhibited improvements in codeine content of 11% and 9% w/w, respectively, compared to PW08-2308.

Although thebaine is the precursor to codeine in the alkaloid synthesis pathway it is considered to be an undesirable “impurity” in poppy straw and latex for the production of codeine as it has negative implications for codeine extraction efficiency and thereby yield. A key criterion is, therefore, to minimize the amount of thebaine in poppy straw harvested for codeine production. The relevant measure of this trait, “tc”, is the amount of thebaine relative to the main alkaloid, codeine (tc=thebaine/codeine). In the 2014/15 season, the tc was 0.09 in PW08-2308, 0.10 in PW11-4118 and 0.09 in PW11-4027. In the present field trials, the mutant ‘Tasman’ lines with the highest codeine content (EM4-0045 and EM3-1204) both had a tc value of 0.06 in the trials. Moreover, both the EM4-0045 light green-yellow leaf mutant line and the EM3-1204 line displayed a substantially reduced level of thebaine relative to codeine compared to the parent line PW08-2308 from which they were derived.

Whilst of the 3 Papaver somniferum commercial lines both PW11-4118 and PW11-4027 exhibited lower thebaine to codeine levels than the PW08-2308, the 3 lines otherwise displayed very similar impurity profiles in the present trials. Other than the altered codeine and thebaine content of the poppy straw of the mutant ‘Tasman’ lines, those lines also showed very similar levels of impurity alkaloids the parent line PW08-2308 from which they were derived.

Papaver somniferum ‘Tasman’ lines generally exhibit even development, high vigour, and early flowering, a near absence of the twisted stem trait, and moderate resistance to down mildew (DM) infection. In the present field trials all but 1 (EM3-0056), exhibited very suitable phenotypes for being grown commercially.

In particular, of the 7 mutant lines included in these trials, EM3-1204, EM3-1217, and EM3-1203 all showed exceptional vigour. Clearly these lines do not contain mutations that have a negative visible impact on plant growth and development. The EM4-0045 (light green-yellow leaf mutant) line in particular showed very good vigour across all trials, and a growth habit that was consistent with the commercially grown parent ‘Tasman’ line (PW08-2308) from which it was derived. This was unexpected given the light-green coloration of the leaves of these plants particularly during the vegetative stages of plant development. This colour difference between the mutant and the parent line was less obvious closer to flowering although it was still readily apparent in flower stems and capsules.

The observed increased codeine content and decreased t/c ratio of the EM4-0045 line relative to the parent line PW08-2308 is consistent with results for the EM4-0045 line from other field trials.

Given the above results it appears the mutation that results in the light green-yellow leaves in the EM4-0045 line does not cause negative pleiotropic effects on plant vigour and further, that this line does not contain other unrelated EMS-induced mutations that visibly affect plant growth, development and/or plant vigour.

### EXAMPLE 5

### Evaluation of Leaf Colour of Papaver Somniferum Line EM4-0045

In addition to alkaloid differences, the EM4-0045 line exhibits a substantially lighter leaf and stem colour in comparison to the parental line, PW08-2308, and other typical commercially-grown P. somniferum ‘Tasman’ lines. This colour difference is highly marked in field grown plants as further illustrated in FIG. 3, which shows a field plot of EM4-0045 (left) growing next to a ‘Tasman’ line (right plot) exhibiting the typical darker green-yellow leaf phenotype in a field trial at Forest, Tasmania, in late 2016. To quantify this colour difference, spectrophotometer analysis was conducted on leaf tissues obtained from planthouse-grown plants. Also included in this analysis was a second high codeine line (EM3-0006) which has a very light green-yellow leaf colour phenotype (see FIG. 5).

### 1. Plant Growing Conditions

Plants were grown in a planthouse at Tasmanian Alkaloids Pty Ltd, Westbury, Tasmania during the 2016 winter season. Seeds were sown on 23 Jun. 2016 in 20 cm diameter pots which contained a potting mix consisting of equal parts peat moss and composted pine bark. Once sown, seeds were covered with a thin (˜0.5 cm) layer of vermiculite and grown under an 18 hour light/8 hour dark photoperiod through use of supplemental lighting (high pressure sodium lamps; Horti Master greenPower, 600 W 400 v, E40). Automated irrigation and climate control systems were used to maintain pot moisture content at 30-40% volume and planthouse day and night temperatures at ˜20° C. and ˜15° C., respectively.

Following germination, seedlings were thinned to 5 plants per pot. A granular slow-release fertiliser was added to each pot on 9 Aug. 2016 (˜1 g of Basacote Plus 3M; COMPO GmbH & Co. KG).

### 2. Tissue Sampling

A single leaf from each of ten individual EM4-0045, EM3-0006 and PW08-2308 plants was analysed by spectrophotometry. The plants from which the leaves were obtained were all healthy and viable with no symptoms of disease or nutrient deficiency. Leaves were abscised at the stem and immediately transferred to the Chemical Research and Development laboratory at Tasmanian Alkaloids Pty Ltd, Westbury, Tasmania for analysis. As Papaver somniferum plants grow, older leaves towards the base of the plant begin to show visible signs of senescence. Therefore, younger leaves occurring towards the top of the stem which showed no visible signs of senescence and which were of a suitable size for spectrophotometer analysis (≥10 cm long×≥5 cm wide) were selected for analysis. For 19 out of 20 EM4-0045 and PW08-2308 plants, the fifth youngest leaf was sampled from plants (e.g., the leaf at the fifth leaf node down from the apical meristem). One PW08-2308 plant was sampled at the fourth youngest leaf due to the fifth leaf being damaged. In comparison to EM4-0045 and PW08-2308 lines, plants of the EM3-0006 line exhibit reduced vigour and delayed plant development. All plants were sampled on 18 Aug. 2016, 57 days after sowing. At this time, plants of EM4-0045 and PW08-2308 lines were 70-90 cm tall and in the early- to mid-hook developmental stage. In contrast, EM3-0006 line plants were shorter and in the running up stage.

The plants sampled for spectrophotometer analysis had been grown as part of a larger study involving two additional lines. In total, the experimental population comprised nine pots of each of five lines sown in a complete randomised block design. To obtain the 10 plants used for spectrophotometer analysis, a single plant was sampled from each of the nine PW08-2308, EM4-0045 and EM3-0006 pots, respectively, with each plant being randomly selected from within each pot. A tenth plant for each line was then obtained by sampling a second plant from one randomly selected pot.

### 3. Spectrophotometer Analysis

Spectrophotometry measurements were performed using a HunterLab UltraScan PRO spectrophotometer (Hunter Associates Laboratory, Virginia, USA). Reflectance (specular included) was measured on the upper-third region of each leaf (adaxial surface) using D65 illumination. Leaves were backed by a white tile and held against the 0.390″ port by the instrument's spring loaded clamp arm. Using reflectance measurements, CIE 1976 L*a*b* and tristmulus XYZ values were calculated using EasyMatch QC software (Hunter Associates Laboratory, Virginia, USA). To obtain the dominant wavelength values for each line, chromaticity x,y coordinates were firstly obtained by calculating CIE xyY values from XYZ values; where x=X/(X+Y+Z) and y=Y/(X+Y+Z). Each of these sample coordinates, in addition to the x,y coordinates of the D65 illuminant (x=0.31382, y=0.33100; CIE 1964 10°), were then plotted on a chromaticity graph drawn in Microsoft Excel 2010 using CIE 1964 10° chromaticity coordinates (0.1 nm interval values; downloaded from http://cvrl.ioo.ucl.ac.uk/). A straight line was then drawn between the x,y coordinates of each line and the illuminant, respectively, with the line extrapolated out so as to intersect with the spectral locus (i.e., the dominant wavelength value).

### 4. Results

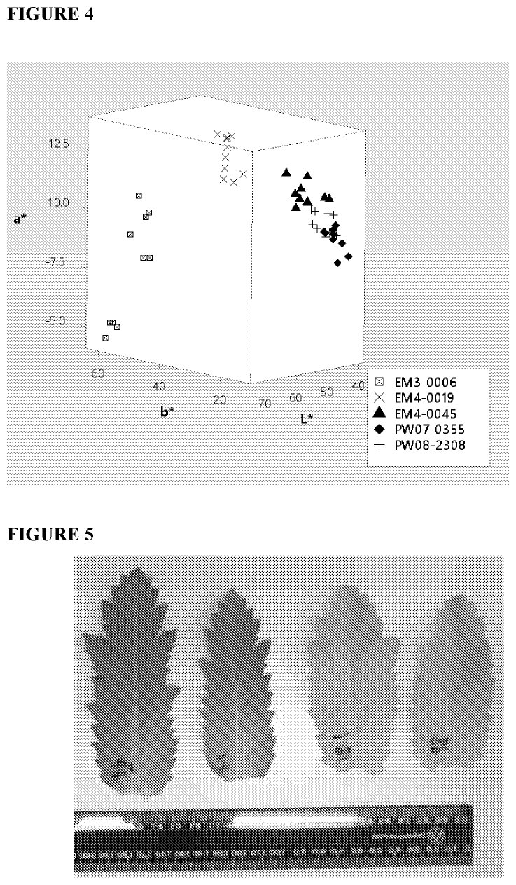

Individual spectrophotometer results (L*a*b* values) and mean dominant wavelength values for each line are shown in FIG. 4 and Table 4, respectively. As seen in the 3-dimensiona plot shown in FIG. 4, all samples of each line grouped within their respective clusters, and spectrophotometry analysis clearly differentiated the EM3-0006 line from both the EM4-0045 and PW08-2308 lines. As expected, this very light green-yellow leaf line (EM3-0006) was detected as being substantially lighter in colour (L*=66.92 vs. 43.82 to 45.25; where L*=0 is black and 100=‘brightest white’) and more yellow (b*=57.698 vs. 18.20 to 22.69; where greater positive values on the b* axis represent higher yellow colour values) in comparison to both PW08-2308 and EM4-0045 lines. These differences were reflected in dominant wavelength values, with the EM3-0006 line having a higher dominant wavelength of 568 nm (Table 4).

The spectrophotometer analysis of these planthouse-grown plants indicates that the leaves of the PW08-2308 and EM4-0045 lines are more similar in colour than when each is compared to the EM3-0006 line. Despite this, the PW08-2308 and EM4-0045 lines were separated on both the a* (range: PW08-2308 −9.36 to −10.38; EM4-0045 −10.48 to −11.88) and b* (range: EM4-0045 19.88 to 26.26; PW08-2308 15.78 to 21.21) axes (see FIG. 4). Further, the PW08-2308 and EM4-0045 lines were detected as having dominant wavelengths of 560 nm and 561 nm, respectively (Table 4).

TABLE 4
Spectrophotometry results (mean values for each line) and dominant wavelength values

| | | | | | | | | | Dominant |
|---|---|---|---|---|---|---|---|---|---|
| | CIE 1976 L*a*b* | | | CIE 1931 XYZ colour space | | | CIE xyY colour space | | wavelength^{a} |
| Line | L* | a* | b* | X | Y | Z | x | y | (nm) |
| PW08-2308 | 43.822 | −9.858 | 18.200 | 11.5824 | 13.7306 | 8.2301 | 0.3453 | 0.4093 | 560 |
| EM4-0045 | 45.250 | −11.040 | 22.690 | 12.3415 | 14.7912 | 7.6749 | 0.3546 | 0.4249 | 561 |
| EM3-0006 | 66.920 | −7.449 | 57.698 | 32.6997 | 36.6827 | 8.3704 | 0.4206 | 0.4718 | 568 |

^{a}Calculated using CIE 1964 10° standard observer and illuminant D65

The lighter leaf colour of EM3-006 plants compared to EM4-0045 plants when these plant lines are grown in a planthouse is shown in FIG. 5.

### 5. Discussion

When grown under common field conditions, the leaves of plants of the EM4-0045 line have a distinct light green-yellow appearance when compared to typical green Papaver somniferum poppy plants such as PW08-2308. Although the colour distinction between the planthouse-grown EM4-0045 and PW08-2308 line plants examined in this study was less pronounced than what is typically observed under field conditions, spectrophotometry analysis identified colour differences and detected the EM4-0045 line as being lighter and more yellow in colour, as evidenced by a dominant wavelength value of 561 nm for EM4-0045 in comparison to 560 nm for PW08-2308 (see Table 4).

The dominant wavelength values indicate that all three lines had leaf colours within the green-yellow colour spectrum. Thus, the three lines can be described as having green-yellow (PW08-2308), light green-yellow (EM4-045) and very light green-yellow (EM3-0006) leaf colours.

### EXAMPLE 6

### Light Green-Yellow Leaf Colour of Papaver Somniferum line EM4-0045 is Associated with Reduced Leaf Chlorophyll and Carotenoid Pigment Content

The colour of planthouse-grown PW08-2308, EM4-0045 and EM3-0006 lines was quantified by spectrophotometry in Example 5 above. The physiological basis of the detected colour differences was examined in the present study by quantification of chlorophyll and carotenoid leaf pigments.

### 1. Tissue Sampling

The same populations of plants as described in above Example 5 were sampled for pigment analysis. The plants were sown in a complete randomized block design with each of three blocks containing one pot per line. Four plants within each pot were selected for sampling, resulting in a total of 12 plants per block, the tissues of which were combined to create ‘pooled’ samples. The EM3-0006 line had low establishment; eight plants in each of Blocks 1 and 2 and seven plants in Block 3, totaling 23 plants in all.

Briefly, the preparation of leaf tissue samples for pigment analysis involved the grinding of fresh tissues in a mortar and pestle under liquid nitrogen followed by freeze-drying and subsequent pigment extraction for HPLC analysis. The sixth youngest leaf was sampled from each plant with tissues being pooled within blocks for each line. Once harvested, pooled leaf tissues were segmented roughly (˜8 pieces per leaf), mixed, and then randomly selected until the desired fresh tissue weight for freeze-drying was obtained (˜11 g).

All plants were sampled when in the early-hook to mid-hook stage. For PW08-2308 and EM4-0045 lines sampling occurred 55-58 days after sowing. The slower development of the EM3-0006 line resulted in plants of this line being sampled approximately one week later (64 days after sowing). Leaves from all 23 sampled plants of the EM3-0006 line were combined into a single sample to obtain the required tissue weight for freeze-drying.

Once ground in liquid nitrogen, tissue samples were freeze-dried on a Christ Alpha 2-4-LD Plus freeze-drier. Four out of seven samples were degraded during freeze-drying and so were excluded from pigment analysis; the excluded samples comprised two EM4-0045 and two PW08-2308 replicates. A second set of leaves were therefore harvested from EM4-0045 and PW08-2308 plants post flowering (70 days after sowing). Here, one randomly selected plant from each of nine EM4-0045 and PW08-2308 pots were sampled and combined within lines, respectively. On this occasion, the fifth youngest leaf was sampled from each plant. Both samples were successfully freeze-dried.

### 2. HPLC Pigment Analysis

Following freeze-drying, 50 mg of ground freeze-dried leaf tissue was moistened with water (100-200 μl) and then extracted in an acetone:methanol (7:3) solution as described in Albert NW, Lewis DH, Zhang H, Irving U, Jameson PE, Davies KM. (2009), Light-induced vegetative anthocyanin pigmentation in Petunia. J Exp Bot 60 (7):2191-2202. doi:10.1093/jxb/erp097. Samples were analysed using a Dionex Ultimate 3000 HPLC system with an Accucore RP C30 column. Absorbance was monitored using a photodiode array detector. Carotenoids and chlorophyll b were detected at 450 nm, while chlorophyll a and other chlorophyll derivatives were monitored at 430 nm. The levels of carotenoids were determined as β-carotene equivalents per gram of dry-weight (DW) of tissue. Chlorophyll a and b, respectively, were determined using chlorophyll a and b standard curves derived from a spinach extract. β-carotene and lutein were identified in the extracts by comparison of retention times and on-line spectral data with standard samples. Trans-β-carotene was purchased from Sigma Chemicals (St Louis, Mo., U.S.A.). Other carotenoids (neochrome, α-carotene, violaxanthin and neoxanthin) were putatively identified by comparison with published retention times and spectral data for carotenoids present in the spinach extract.

### 3. Results

The number of samples in this study was reduced due to the degradation of samples during freeze-drying. Ultimately, a single sample for each of EM3-006, EM4-0045 and PW08-2308 was obtained at the hook timepoint. Furthermore, single samples were obtained for EM4-0045 and PW08-2308 at post flowering. Despite this, a marked decrease in leaf plant pigment content was clearly seen in the EM3-0006 line. This line contained less than 10% of the total chlorophyll present in the green-yellow progenitor line PW08-2308. A substantial reduction in carotenoid content was also observed in EM3-0006. Of interest, the lutein content of EM3-006 (341.6 μg^{-g} DW) exceeded the total chlorophyll a and chlorophyll b content of this line (317.1 μg^{-g} DW; see Table 5 below). The severe reduction of chlorophyll in EM3-0006 leaves may ‘unmask’ the yellow-coloured lutein pigment in the leaves of this line; thereby contributing to the very light green-yellow leaf colour observed.

The light green-yellow EM4-0045 line was also found to contain reduced levels of leaf chlorophyll; represented by ˜10-18% reductions in total chlorophyll content at both early hook and post flowering timepoints. A slight decrease in carotenoid pigments (˜4-5%) relative to the green-yellow progenitor line PW08-2308 was also observed for the EM4-0045 line.

The results are shown in Table 5. Values are presented in the table on a μg^{-g} dry-weight (DW) basis. Numbers in parentheses represent the percentage of total pigments (within each pigment class), and percentages relative to PW08-2308 values are calculated for lines within timepoints. H=hook, PF=post flowering and μ-car=μ-carotene.

TABLE 5
Chlorophyll and carotenoid content of planthouse-grown P. somniferum

| | | | Chlorophylls | | | | | Carotenoids | | | | |
|---|---|---|---|---|---|---|---|---|---|---|---|---|
| | | | | | | | % of | | | | | % of |
| Line and | | Pool | | | a/b | | PW08- | | | | | PW08- |
| timepoint | Leaf colour | size | CHL a | CHL b | ratio | Total | 2308 | Lutein | β-car | Other | Total | 2308 |

Hook

| | | | | | | | | | | | | |
|---|---|---|---|---|---|---|---|---|---|---|---|---|
| EM3-0006 | Very Light | 23 | 255.0 | 62.1 | 4.1 | 317.1 | 7.7 | 341.6 | 171.5 | 64.0 | 577.1 | 63.0 |
| | Green-yellow | | (80.4) | (19.6) | | | | (59.2) | (29.7) | (11.1) | | |
| EM4-0045 | Light Green- | 10 | 2854.6 | 836.3 | 3.4 | 3690.9 | 89.7 | 592.9 | 199.9 | 92.8 | 885.6 | 96.7 |
| | yellow | | (77.3) | (22.7) | | | | (66.9) | (22.6) | (10.5) | | |
| PW08-2308 | Green-yellow | 12 | 3192.0 | 924.8 | 3.5 | 4116.8 | | 611.4 | 209.3 | 95.5 | 916.2 | |
| | | | (77.5) | (22.5) | | | | (66.7) | (22.8) | (10.4) | | |

Post flowering

| | | | | | | | | | | | | |
|---|---|---|---|---|---|---|---|---|---|---|---|---|
| EM4-0045 | Light Green- | 9 | 3296.9 | 963.6 | 3.4 | 4260.5 | 81.6 | 621.2 | 296.3 | 91.0 | 1008.5 | 94.6 |
| | yellow | | (77.4) | (22.6) | | | | (61.6) | (29.4) | (9.0) | | |
| PW08-2308 | Green-yellow | 9 | 4025.2 | 1199.2 | 3.4 | 5224.4 | | 716.3 | 247.6 | 102.2 | 1066.1 | |
| | | | (77.0) | (23.0) | | | | (67.2) | (23.2) | (9.6) | | |

### EXAMPLE 7

### Lime-Green Leaf ‘Ted’ Papaver Somniferum line EM4-0019 Having a Thebaine Chemotype

### 1. Mutagenesis of ‘Ted’ P. Somniferm and Field Trials

The seed of a stably reproducing P. somniferum ‘Ted’ high-thebaine parent line, PW07-0355, which produces thebaine as the predominant alkaloid as described in WO 2009/109012 was subjected to EMS mutagenesis treatment and the resultant bulked M2 seed was sown in a field screening trial as described in Example 1 for the ‘Tasman’ P. somniferum plants.

As in Example 1, M2 Ted plants were inspected regularly throughout development to screen for interesting and potentially useful phenotypes. At early stages of development plants with phenotypes of interest were marked with a flag to ensure they were examined further. After ‘running up’, all of these plants were tagged with a label that described their phenotype. All labelled phenotypic mutants were self-pollinated by placing and securing a paper bag over the primary flower bud prior to anthesis. M3 seed from each of the M2 plants was harvested separately when plants were fully mature and dry (February 2012) and were assigned line numbers starting with EM3 or EM4.

EM4-0019 was the line number assigned to M3 seed harvested from a plant that displayed a lime-green leaf colour rather than the typical green-yellow seen in the wild-type parent line. This line, amongst others, was entered into a M3 Disease resistance/phenotype screening trial (described above in Example 3 “M3 Field trials”) in the 2012/13 field season to determine if the altered phenotypes resulted in any improvement in resistance to, or tolerance of, downy mildew (Peronospora meconosidis, previously known as Peronospora arborescens) infection. There was no expectation that altered phenotypes of the mutant lines would result in an increase in alkaloid content, so the TED parent line (PW07-0355) was not entered in the trial for comparison. However, a high-thebaine sibling of PW07-0355, PW07-0358, was included, along with a high-thebaine line, PH10-1561, that was grown commercially in that season. In addition to its lime-green leaf colour, EM4-0019 surprisingly exhibited an elevated thebaine content relative to the other Ted lines include in this trial.

TABLE 6
Thebaine content of poppy straw from P. somniferum Ted lines
in the 2012/2013 disease resistance/phenotype screening trial

| | | Thebaine |
|---|---|---|
| Line | Leaf colour | (% w/w) |
| PW07-0358 | Green | 3.29 |
| PH10-1561 | Green | 3.68 |
| EM4-0019 | Lime-green | 4.31 |

The results obtained in the M3 disease resistance/phenotype screening trial, warranted additional follow-up and EM4-0019 was entered in the 2013/14 season multi-location variety trials, which were aimed at identifying high-thebaine TED lines with wide adaptability to different growing regions. These trials contained 30 entries including 4 high thebaine ‘TED’ P. somniferum lines that were commercially grown at that time, namely PW08-0417, PH10-1561, PH10-1575 and PW11-5603. The trials were sown across a wide geographical distribution (Tunbridge, Ross, Penguin, Bracknell, and Moriarty), representing a diverse range of poppy growing regions in Tasmania, Australia. Each trial was sown within a poppy paddock and was treated the same as the surrounding poppy crop in regards to herbicide, fertilizer, fungicide and irrigation treatments. Trial plants were not sprayed with any plant growth regulators.

Plants were allowed to mature and dry under field conditions. Immediately prior to the harvest of the surrounding commercial crop, all capsules from within a designated quadrat were hand-harvested from each trial plot. The harvested straw samples therefore consisted only of plant capsules and seed and contained no plant stems. All harvested material was stored for up to one month before being threshed to remove seed. These poppy straw samples were ground, and the ground straw was extracted in 2% acetic acid and 10% ethanol in distilled water as described above. An aliquot of the extract was filtered prior to UPLC analysis of alkaloid content and alkaloid profile as also described above.

For each individual trial site, the alkaloid (% w/w) results for the three replicates of each M3 line were analysed in Agrobase Generation II (Agronomix Software) using “GLM” analysis to produce a mean value for each line at that trial site. An overall trial mean for each alkaloid (% w/w), from across the 5 trial sites, was then determined in Agrobase Generation II using the GxE Analysis function “ANOVA-combined RCBD: ENV. X ENTRY model. The overall trial means for thebaine content in these trials is shown in Table 7. As seen, the lime-green line EM4-0019 produced substantially more thebaine in comparison to the four commercial Ted lines; all of which had the wild-type green-yellow leaf colour. The improved thebaine content of EM4-0019 represented a 9.67-12.09% increase over the four commercial lines being grown at that time.

TABLE 7
Thebaine content of poppy straw from P. somniferum ‘Ted’
lines in 2013/2014 multi-location variety trials

| | Thebaine |
|---|---|
| Line | (% w/w) |
| PW08-0417 | 3.623 |
| PH10-1561 | 3.596 |
| PH10-1575 | 3.589 |
| PW11-5603 | 3.668 |
| EM4-0019 | 4.023 |

2. Capsule Alkaloids Obtained from Planthouse Grown Plants

The Ted lime-green line (EM4-0019) and its' progenitor green-yellow line (PW07-0355) were sown on 20 May 2015 at Tasmanian Alkaloids, Westbury, Tasmania and grown in a planthouse. Plant growing conditions were as described in Example 5. Eleven pots of each line were grown with pots thinned to 6 plants per pot on 9 Jun. 2015.

For each line, 54 plants were randomly selected and randomly assigned to one of 9 replicates (each containing 6 plants). Capsules were harvested at the dry-capsule stage following plant maturation and desiccation. Following the removal of seeds, the six capsules of each replicate were combined and ground using a coffee/spice grinder. Two grams of the ground capsule tissue was then measured and alkaloids extracted in 40 ml extractant solution (2% acetic acid 10% ethanol). The extraction and UPLC methods used were as described above.

### 3. Results

Mean values were calculated for each line with results showing that plants of the lime-green line EM4-0019 accumulated a greater amount of thebaine on average (2.997% DW) relative to the green-yellow coloured progenitor line PW07-0355 (2.875% DW). This represented a 4.2% increase in capsule thebaine content.

### 4. ‘Ted’ Spectrophotometer Data

Spectrophotometry analysis was similarly conducted for Ted EM4-0019 and PW07-0355 lines as described above for the Tasman lines in Example 5. The Ted data was collected at the same time as the ‘Tasman’ spectrophotometer data with Ted plant growing conditions and analysis also as per Example 5. A sampling error resulted in only nine PW07-0355 leaf samples being analysed (ten leaf samples were analysed for all other Ted and Tasman lines).

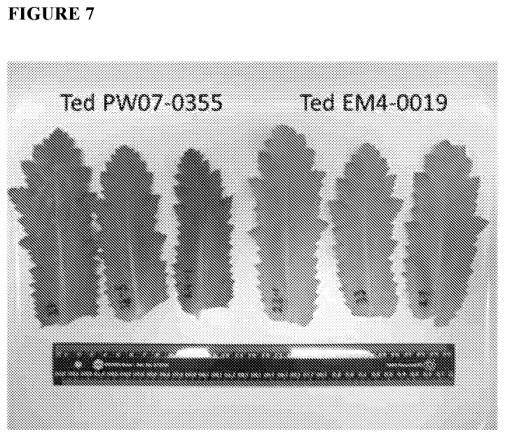

The spectrophotometer results for Ted lines are set out in Table 8 below and a three-dimensional plot of L*a*b* values for the nine PW07-0355 leaf samples and each of ten Ted EM4-0019 line leaf samples from planthouse grown plants are shown in FIG. 7. For comparison purposes this figure also contains the L*a*b* values of the individual leaf samples examined for each of the three Tasman lines (PW08-2308, EM4-0045 and EM3-0006). FIG. 7 shows three leaves each of the Ted PW07-0355 and EM4-0019 lines.

The Ted progenitor line PW07-0355 was found to have an equivalent dominant wavelength to the similarly coloured Tasman green-yellow line PW08-2308 (both 560 nm; see Table 8 and Example 5). Thus, the PW07-0355 line can also be described as being green-yellow in colour. As expected, the Ted line EM4-0019 was found to have to have a higher dominant wavelength (564 nm; Table 8) indicating that the leaf colour of this line is further towards the yellow end of the green-yellow colour spectrum. ‘Lime green’ is a pure spectral colour at approximately 564 nm, and thus, the leaves of the Ted EM4-0019 line are described herein as being lime-green in colour.

TABLE 8
Spectrophotometry results (mean values for each line) and dominant wavelength values

| | | | | | | | | | | Dominant |
|---|---|---|---|---|---|---|---|---|---|---|
| | | CIE 1976 L*a*b* | | | CIE 1931 XYZ colour space | | | CIE xyY colour space | | wavelength^{a} |
| Line | n | L* | a* | b* | X | Y | Z | x | y | (nm) |

Ted

| | | | | | | | | | | |
|---|---|---|---|---|---|---|---|---|---|---|
| PW07-0355 | 9 | 42.588 | −9.244 | 15.642 | 10.936 | 12.900 | 8.354 | 0.340 | 0.4007 | 560 |
| EM4-0019 | 10 | 56.172 | −12.544 | 36.968 | 20.203 | 24.104 | 9.001 | 0.379 | 0.4522 | 564 |

^{a}Calculated using CIE 1964 10° standard observer and illuminant D65

### 5. Chlorophyll and Carotenoid Pigment Content of P. Somniferum ‘Ted’ Lines EM4-0019 and PW07-0355

Chlorophyll and carotenoid pigment analysis was also performed for Ted lime-green EM4-0019 and Ted green-yellow PW07-0355. This analysis was undertaken at the same time as described for Tasman lines in Example 6. Ted plant growth conditions, tissue sampling and tissue processing, and pigments analyses were also as reported in Example 6. Two replicates were obtained for PW07-0355 at the ‘hook’ timepoint and the mean values calculated for these two replicates are presented in Table 9. All other data presented in this table are based on single replicates.

The pigment profile of the green-yellow Ted line PW07-0355 was remarkably similar to that of the Tasman green-yellow line PW08-2308. For example, both green-yellow lines were found to have similar total chlorophyll content (Tasman 4116-5224 μg^{-g} DW, Ted 4541-5305 μg^{-g} DW) and chlorophyll a/b ratios (Tasman 3.4-3.5, Ted 3.6-3.9), and near-identical total carotenoid contents (Tasman 916-1066 μg^{-g} DW, Ted 922-1106; ranges taken from Table 5 and Table 9, respectively). The lime-green Ted line (EM4-0019) had substantially reduced pigment content. Reductions of ˜50-60% in total chlorophyll content and ˜35-55% in total carotenoid content were observed when compared to the parent line PW07-0355 across the two examined timepoints (Table 9).

TABLE 9
Chlorophyll and carotenoid content (μg^{-g} DW) of planthouse-grown ‘Ted’ P. somniferum

| | | | Chlorophylls | | | | | | Carotenoids | | | |
|---|---|---|---|---|---|---|---|---|---|---|---|---|
| | | | | | | | % of | | | | | % of |
| Line and | | Pool | | | a/b | | PW07- | | | | | PW07- |
| time point | Leaf colour | size | CHL a | CHL b | ratio | Total | 0355 | Lutein | β-car | Other | Total | 0355 |

Hook

| | | | | | | | | | | | | |
|---|---|---|---|---|---|---|---|---|---|---|---|---|
| EM4-0019 | Lime-green | 12 | 1840.7 | 351.4 | 5.2 | 2192.1 | 48.3 | 328.7 | 9.0 | 66.1 | 403.8 | 43.8 |
| | | | (84.0) | (16.0) | | | | (81.4) | (2.2) | (16.4) | | |
| PW07-0355 | Green-yellow | 12, 12* | 3612.6 | 928.7 | 3.9 | 4541.3 | | 636.4 | 186.8 | 99.3 | 922.5 | |
| | | | (79.5) | (20.5) | | | | (69.0) | (20.2) | (10.8) | | |

Post flowering

| | | | | | | | | | | | | |
|---|---|---|---|---|---|---|---|---|---|---|---|---|
| EM4-0019 | Lime-green | 9 | 1681.3 | 464.6 | 3.6 | 2145.9 | 40.4 | 407.7 | 239.8 | 66.0 | 713.5 | 64.5 |
| | | | (78.3) | (21.7) | | | | (57.1) | (33.6) | (9.2) | | |
| PW07-0355 | Green-yellow | 9 | 4155.0 | 1150.7 | 3.6 | 5305.8 | | 757.8 | 247.9 | 100.3 | 1106.0 | |
| | | | (78.3) | (21.7) | | | | (68.5) | (22.4) | (9.1) | | |

*Two replicates processed for PW07-0355 ‘hook’ timepoint

### EXAMPLE 8

### Mutation of a Magnesium Chelatase Subunit I (MgChlI) Gene in Light Green-Yellow Papaver Somniferum Line EM4-0045

To investigate the genetic and physiological basis of the light green-yellow phenotype exhibited by EM4-0045, a comparative transcriptome study was undertaken to examine differential gene expression between EM4-0045 and its' green-yellow progenitor line PW08-2308.

### 1. Genetic Investigation of EM4-0045 Line

Seed of both EM4-0045 and PW08-2308 lines were sown in a greenhouse at Tasmanian Alkaloids, Westbury, Tasmania on 30 May 2015. Twenty-two pots were sown per line with all pots thinned to six plants per pot following seedling emergence. Plants of both lines were randomly selected for RNA sampling with plant tissues being sampled at three timepoints: 4-leaf stage (23 days after sowing; DAS), 8-leaf stage (26 DAS) and Early Hook (55 DAS). On each occasion, the youngest, fully-expanded leaf was sampled; with ‘leaf 1’ being the first true leaf after the dicotyledonous leaves. Each RNA replicate contained leaf tissues sampled from six individual plants. Thus, 18 individual plants were sampled at each time-point for each line (6).

TABLE 10
Transcriptome study design and date of tissue
sampling (DAS = days after sowing)

| | | | Date of sampling | Number of |
|---|---|---|---|---|
| | Time- | Developmental | (DAS) | replicates |
| Line | point | stage | (dd/mm/yy) | (n) |
| EM4-0045 | 1 | 4 leaf | 22/6/15 (22) | 3 |
| EM4-0045 | 2 | 8 leaf | 25/6/15 (25) | 3 |
| EM4-0045 | 3 | Early hook | 24/7/15 (55) | 3v |
| PW08-2308 | 1 | 4 leaf | 22/6/15 (22) | 3 |
| PW08-2308 | 2 | 8 leaf | 25/6/15 (25) | 3 |
| PW08-2308 | 3 | Early hook | 24/7/15 (55) | 3 |

RNA isolation involved the grinding of plant tissues in a mortar and pestle under liquid nitrogen followed by isolation using a RNeasy Plant Mini Kit as per the manufacturer's protocol (Qiagen, Hilden, Germany). RNA quality was quantified on an Agilent 2100 Bioanalyzer (Agilent Technologies, Santa Clara, USA) with the RNA integrity number (RIN) of all samples found to exceed the acceptable quality threshold for sequencing (RINs ranged between 7.5 to 8.4). For each sample, 5 μg RNA was shipped to the Australian Genome Research Facility Ltd (AGRF) for sequencing. Following the preparation of individual mRNA-seq libraries (100bp paired-end) sequencing was undertaken on an Illumina HiSeq 2000 instrument. Five sequencing lanes generated a total of 619 and 660 million sequencing reads for PW08-2308 and EM4-0045 lines, respectively.

The raw sequencing reads were firstly screened for adapters and quality trimmed (cut-off score of 28) using fastq-mcf (ea-utils.1.1.2-806; Aronesty E (2011) ea-utils: command-line tools for processing biological sequencing data. https://github.com/ExpressionAnalysis/ea-utils). Thereafter an in-house perl script was used to trim 15 bases off the 5′ end of sequences and remove reads with Ns and mononucleotides. Once this pre-processing was complete, reads were mapped to a proprietary Papaver somniferum genome sequence assembly using bowtie2 (version 2.2.5; Langmead, B, Trapnell, C, Pop, M and Salzberg, SL (2009) Ultrafast and memory-efficient alignment of short DNA sequences to the human genome. Genome Biology, 10:R25, https://doi.org/10.1186/gb-2009-10-3-r25). The genotype used for the sequencing and assembly of the P. somniferum genome sequence was a morphine-producing (i.e, wild-type) cultivar (line PH11-0943). The resulting SAM files obtained from the mapping of RNA sequence data to the genome sequence were converted to BAM files and sorted using SAMtools (version 0.1.18; Li, H et al. (2009) The sequence alignment/map format and SAMtools. Bioinformatics, 25(16), 2078-79. DOI:10.1093/bioinformatics/btp352). The BAM files were then used to generate raw read counts for P. somniferum gene models using HTseq (version 0.6.1; Anders S, Pyl PT and Huber W (2015) HTSeq—a Python framework to work with high-throughput sequencing data. Bioinformatics, 31(2), 166-169. https://doi.org/10.1093/bioinformatics/btu638); with ‘strandedness’ set to ‘reverse’ given the RNASeq data was stranded data. Thereafter, differentially expressed genes (DEGs) were identified using DESeq2 (version 1.12.2; Love, MI, Huber, W and Anders, S (2014) Moderated estimation of fold change and dispersion for RNA-Seq data with DESeq2. bioRxiv doi:10.1101/002832) for each of the three timepoint comparisons; these analyses being conducted in R (3.3.0; R Core Team (2013). R: a language and environment for statistical computing. R Foundation for Statistical Computing, Vienna, Austria. http://www.R-project.org/).

Gene model descriptions were subsequently merged with DESeq2 results (csv files) using a custom perl script. The descriptions for P. somniferum gene models had been previously assigned based on results (e.g., BLAST metrics) obtained from a series of BLAST searches of the models against gene databases; including SwissProt (https://www.ebi.ac.uk/uniprot; Bairoch A and Apweiler R (2000) The Swiss-PROT protein sequence database and its supplement TrEMBL in 2000. Nucleic Acids Research, 28(1), 45-48), Uniref90 (http://www.uniprot.org/; Suzek, BE, Huang, H, McGarvey, P, Mazumder, R and Wu, CH (2007) UniRef: comprehensive and non-redundant UniProt reference clusters. Bioinformatics, 23, 1282-1288), RefSeq (https://www.ncbi.nlm.nih.gov/refseq/; Pruitt KD, Tatusova T, Klimke W, Maglott DR (2009) NCBI Reference Sequences: current status, policy and new initiatives. Nucleic Acids Research, 37: D32-D36), Arabidopsis thaliana (Arabidopsis) proteins (TAIR10 http://www.arabidopsis.org/; Lamesch P et al. (2011) The Arabidopsis Information Resource (TAIR): improved gene annotation and new tools. Nucleic Acids Research, 40, doi: 10.1093/nar/gkr1090) and the NCBI non-redundant (NR) DNA database (https://www.ncbi.nlm.nih.gov/home/download/).

In the analysis of DESeq2 results, the main parameters considered were DEG significance values (p-values adjusted for multiple testing; ‘padj’), the magnitude of differential expression (expressed as ‘log2-fold change’), and the read counts of individual genes (average of three replicates) for EM4-0045 and PW08-2308 genotypes. Applying a padj value of ≤1.0e-5, the most significant DEGs at 4 leaf, 8 leaf and Running Up timepoints comprised 39, 16 and 27 genes, respectively. Only eight genes were detected within the ‘top DEGs’ (padj ≤1.0e-5 threshold) at all three timepoints. Within this group, TAgene201937 annotated as a ‘Magnesium chelatase subunit ChlI gene’ was identified as being of high interest. This gene was the most significantly differentiated gene in all timepoint comparisons and was significantly down-regulated in EM4-0045 relative to PW08-2308 at all three developmental stages (log2fold change value range: −1.44 to −1.77). A BLASTp search of the non-corrected TAgene201937 gene model protein against TAIR10 returned a best hit (1.0e-144) for the Arabidopsis magnesium chelatase subunit I gene AT5G45930.1; thereby supporting the functional annotation of the P. somniferum gene model.

As described above, the RNA sequencing data of each genotype and timepoint replicate was mapped to the P. somniferum genome sequence. The mapped reads were displayed as individual tracks on a genome sequence browser, allowing for a comparison between both the RNA sequence of EM4-0045 and PW08-2308 genotypes and the genomic sequence of the wild-type genome sequence line PH11-0943. A manual inspection of RNA-seq mapped to the TAgene201937 gene model identified an EMS-type single nucleotide polymorphism (SNP) in the EM4-0045 genotype. The progenitor PW08-2308 genotype contained the wild-type base at this same position. The putative EMS SNP (cytosine (C) to thymine (T) mutation) identified in the EM4-0045 genotype was predicted to create a premature STOP codon within the predicted protein sequence of TAgene201937. Based on this finding, the known role of magnesium chelatase in chlorophyll biosynthesis and the similar light-green to yellow phenotypes exhibited by magnesium chelatase subunit I knockout/mutants (e.g., Campbell et al. (2015) Identical substitutions in magnesium chelatase paralogs result in chlorophyll-deficient soybean mutants. doi: 10.1534/g3.114.015255; Huang Y-S and Li H-M (2009) Arabidopsis CHLI2 can substitute for CHLI1. Plant Physiology, 150, 636-645; Luo T et al. (2013) Virus-induced gene silencing of pea CHLI and CHLD affects tetrapyrrole biosynthesis, chloroplast development and the primary metabolic network. Plant Physiology and Biochemistry, 65, 17-26) sequencing of TAgene201937 was undertaken to confirm the putative SNP.

### 2. Sequencing of P. Somniferum Magnesium Chelatase Subunit ChlI Genes

Having identified the TAgene201937 gene as a mutational candidate for the light green-yellow phenotype of EM4-0045, the poppy genome sequence was investigated for the presence of other magnesium chelatase subunit I genes (hereafter designated CHLI). The I subunit of magnesium chelatase is encoded by two genes in Arabidopsis (ChlI1 AT4G18480 and ChlI2 AT5G45930; Huang and Li 2009). These 424aa (AT4G18480) and 418aa (AT5G45930) protein sequences share 83% similarity at the protein level and were TBLASTN searched against the P. somniferum genome sequence. Two CHLI-like genes were identified, the TAgene201937 mutational candidate (hereafter referred to as PsCHLI-A) and a second CHLI gene designated TAgene224147 (hereafter referred to as PsCHLI-B). In the transcriptome study discussed above, PsCHLI-B was similarly expressed in EM4-0045 and PW08-2308 genotypes at all timepoints, and was thus, not detected as being differentially expressed.

Sequencing of genomic DNA (gDNA) and complementary DNA (cDNA) was undertaken for both PsCHLI-A and PsCHLI-B genes. Four lines were examined including the wild-type genotype used for construction of the P. somniferum genome sequence (PH11-0943), Tasman green-yellow (PW08-2308) and Tasman light green-yellow genotypes (EM4-0045) and a Ted (thebaine accumulating) line which was used a parent in the F2 pedigree described below in Example 10. To confirm transcript structure, cDNA sequencing was conducted for PH11-0943 and PW08-2308 lines (Table 11).

TABLE 11
Line and sequencing details for PsCHLI gene sequencing

| | | | TAgene201937 | | TAgene224147 | |
|---|---|---|---|---|---|---|
| P. somniferum | Chemotype* | Sequencing | (PsCHLI-A) | | (PsCHLI-B) | |
| (Line) | (Trade name) | rationale | gDNA | cDNA | gDNA | cDNA |
| PH11-0943 | Morphine | Wild-type line | ✓ | ✓ | ✓ | ✓ |
| | | used for genome | | | | |
| | | sequencing | | | | |
| PW08-2308 | Codeine | Green-yellow | ✓ | ✓ | ✓ | ✓ |
| | (Tasman) | progenitor line | | | | |
| EM4-0045 | Codeine | Light green-yellow | ✓ | — | ✓ | — |
| | (Tasman) | mutant | | | | |
| PW13-4611 | Thebaine | Ted line used in | ✓ | — | ✓ | — |
| | (Ted) | ‘Ted × Tasman’ | | | | |
| | | pedigree^{#} | | | | |

*Predominant morphinan alkaloid in mature poppy capsules;
^{#}see Example 8

Three individuals were sequenced for each line. RNA for cDNA synthesis was isolated as described above. A QuantiTect Reverse Transcription kit (Qiagen, Hilden, Germany) was used to synthesise cDNA as per the manufacturer's protocol. A DNeasy Plant Mini kit (used in accordance with the manufacturer's protocol; Qiagen, Hilden, Germany) was used to isolate plant DNA following the disruption of plant cells through the grinding of leaf tissue under liquid nitrogen in a mortar and pestle.

The P. somniferum genome sequence was used for gDNA and cDNA primer design (Table 12). Primers were located within putative 5′ and 3′ untranslated regions (UTR), targeting non-homologous bases between PsCHLI genes.

TABLE 12
Primer sequences used for amplifying PsCHLI
gDNA and cDNA

| | | | SEQ |
|---|---|---|---|
| | Primer | | ID |
| Gene | Direction | Sequence 5′ to 3′ | NO: |
| gDNA | | | |
| TAgene201937 | F | ATAGAACAAATTGACGGAA | 13 |
| | | TTGTACTTCC | |
| | R | AGGAAAAACTAGGATTAAG | 14 |
| | | AGAACACGAGC | |
| TAgene224147 | F | GCAAAAGATTGACGGAATT | 15 |
| | | GTACTTCATC | |
| | R | CAGGAAAAACTAGGATTAA | 16 |
| | | GAGAACATGAGA | |
| cDNA | | | |
| TAgene201937 | F | GCAAAATCTGTTAACAGCA | 17 |
| | | AAAGATAGAACAA | |
| | R | CCTCGTGATTAGAGTTTGT | 18 |
| | | TTCTGAATTCA | |
| TAgene224147 | F | GCAAAAGATTGACGGAATT | 19 |
| | | GTACTTCATC | |
| | R | CAGGAAAAACTAGGATTAA | 20 |
| | | GAGAACATGAGA | |

High fidelity polymerase chain reaction (PCR) amplification was undertaken using iProof PCR kit reagents (Bio-Rad Laboratories, California, USA). The final reaction volume totalled 25 μL; comprising 5 μL 5× reaction buffer, 0.25 μL dNTP mix (10 mM), 1.2 μL each of forward and reverse primer (10 μM), 0.2 μL iProof polymerase, 16.15 μL ddH₂O and 1 μL template. Cycling conditions were: an initial denaturation step of 98° C. for 30 seconds; 30 cycles of 98° C. for 10 seconds, 57° C. for 10 seconds, 72° C. for 60 seconds; and a final extension step of 72° C. for 5 minutes. Reaction products were run on 1% agarose gels in 0.5×TBE at 120V for 40 minutes. Bands corresponding to the predicted amplicon size (e.g. ˜1,750bp for PsCHLI-A gDNA sequences) were excised and extracted using a Zymoclean Gel DNA Recovery kit (Zymo Research, California, USA) as per the manufacturer's protocol. PCR products were then ligated into pJET 1.2 blunt cloning vectors using a CloneJET PCR Cloning Kit (Thermo Fisher Scientific, Massachusetts, USA) as per the manufacturer's protocol. Ligations were performed at room temperature for lh in a reaction volume of 10 μL. A total of 3 μL of the ligation mix was then used to transform Escherichia coli (DH5-Alpha) by heat shock at 42° C. for 40 seconds with 2 minutes recovery on ice. Transformations were grown in 0.5 mL LB for 90 min at 37° C. on a shaking incubator before plating onto LB+ ampicillin plates (100 μg/mL). Plates were grown overnight at 37° C. with colonies screened by colony PCR using gene specific 5′ and 3′ primers. PCR products were then run on a 1% agarose 0.5×TBE gel to determine fragment size. Colonies yielding inserts of predicted size were grown overnight in 1.5 mL LB media containing 100 μg/mL ampicillin in a shaking incubator at 37° C. Plasmid DNA preps were then made using a Purelink Quick Plasmid Miniprep kit as per the manufacturer's instructions (Thermo Fisher Scientific, Massachusetts, USA). Finally, sequencing was performed by Macrogen (Seoul, South Korea) using pJET forward and reverse vector primers.

The forward and reverse nucleotide sequence files were edited and contigs created using the Contig Express function within Vector NTI (Thermo Fisher Scientific, Massachusetts, USA). Sequence alignments were performed in Geneious 7.1.9 (Biomatters, Auckland, New Zealand) using the Geneious alignment tool and default settings (gap open penalty=12, gap extension penalty=3).

### 3. Gene Sequencing Results—Tagene201937 (PsCHLI-A)

### 3.1 Wild-Type Line PH11-0943

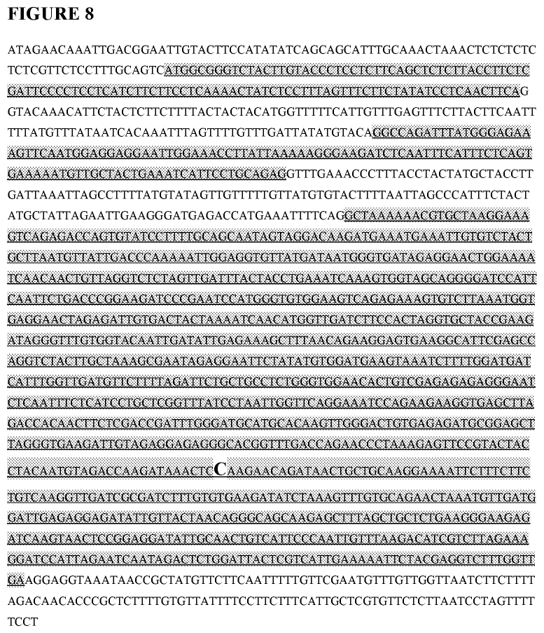

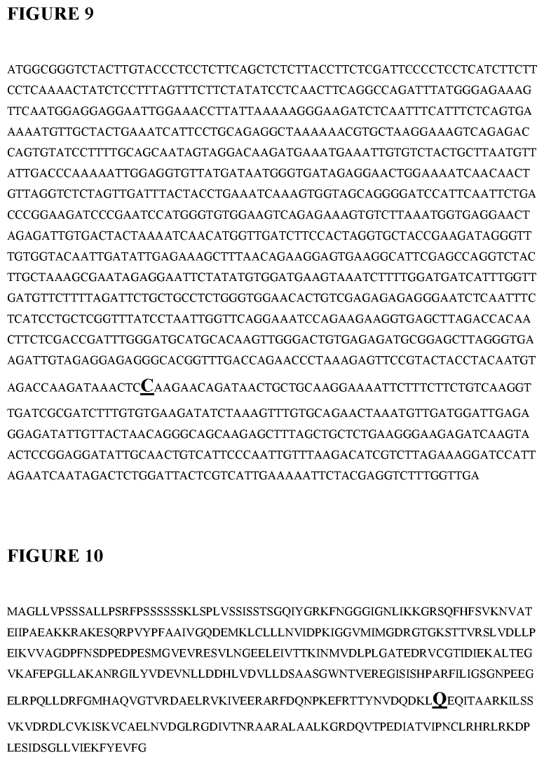

A 1,750bp consensus gDNA sequence was obtained for the wild-type genotype PH11-0943 (SEQ ID NO: 1; FIG. 8). This sequence was found to be 100% identical to the P. somniferum genome assembly sequence. Alignment of the PH11-0943 cDNA sequence (SEQ ID NO: 2; 1,278bp) (FIG. 9) against the gDNA sequence identified three exonic regions within PsCHLI-A. Genetic features of the PsCHLI-A gDNA sequence are presented below in Table 13.

TABLE 13
TAgene201937 (PsCHLI-A) gene features of the wild-
type gDNA sequence obtained in line PH11-0943

| SEQ ID NO: 1 | Coordinates | Length |
|---|---|---|
| (Feature) | (Nucleotide No.) | (Nucleotides) |
| 5′ UTR | 1-85 | 85 |
| Exon 1 | 86-196 | 111 |
| Intron 1 | 197-310 | 114 |
| Exon 2 | 311-427 | 117 |
| Intron 2 | 428-565 | 138 |
| Exon 3 | 566-1,615 | 1,050 |
| 3′ UTR | 1,616-1,750 | 135 |

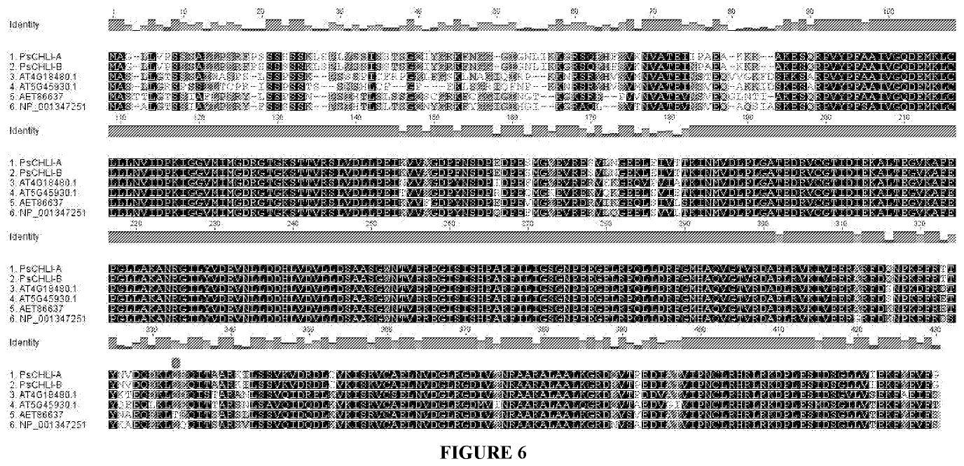

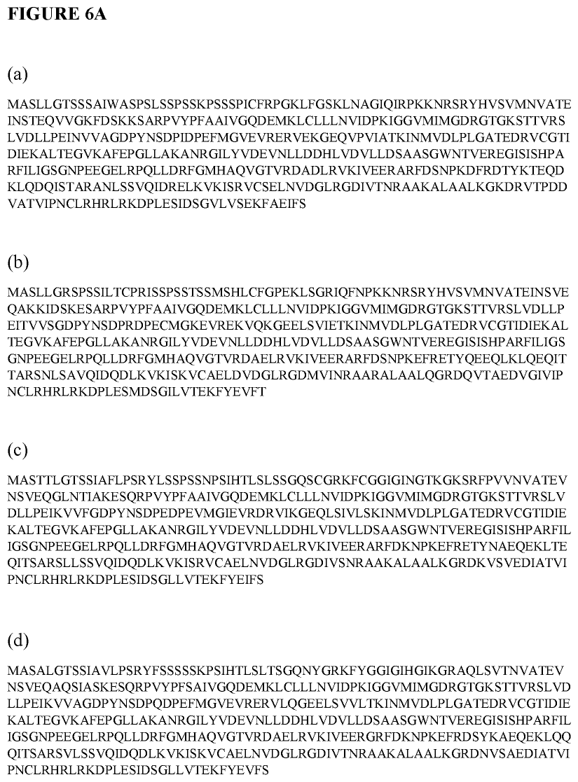

The predicted 425 amino acid (aa) sequence of PsCHLI-A (SEQ ID NO: 3; FIG. 10) returned multiple high similarity matches (>80% identity and ≥99% coverage) to magnesium chelatase subunit I genes when BLASTP searched in the NCBI database. This high sequence similarity and gene homology is illustrated in FIG. 6 which shows a protein sequence alignment between PsCHLI-A with several other plant species CHLI sequences.

### 3.2 Tasman EM4-0045 Light Green-Yellow Mutant

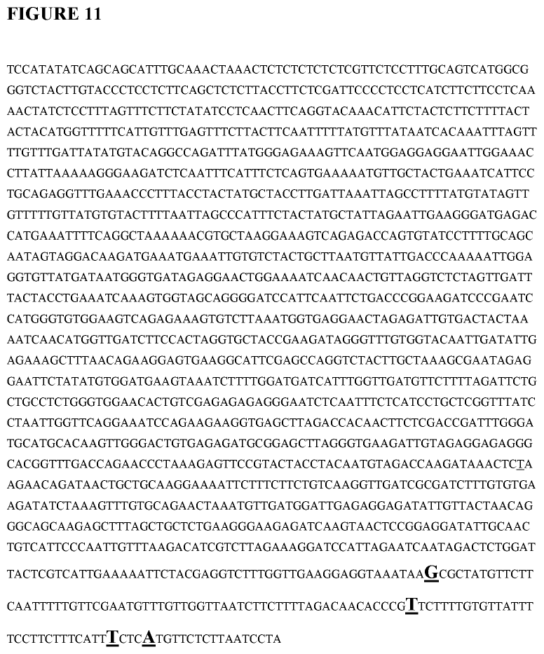

The EMS-type SNP (C to T change) identified through comparison of mapped EM4-0045 line RNA sequence data to the P. somniferum genome assembly sequence was confirmed by sequencing (see SEQ ID NO: 4; FIG. 11). This SNP occurred within the third exon of PsCHLI-A (nucleotide position 1,319 of the wild-type gDNA sequence (SEQ ID NO: 1; FIG. 8) and was confirmed to create a predicted stop codon at position 328 within the PsCHLI-A protein (CAA to TAA codon change; Q328*). This premature truncation is expected to result in a non-functional PsCHLI-A protein and thereby disrupt chlorophyll biosynthesis.

Four additional SNPs were identified in the 3′ UTR of the EM4-0045 PsCHLI-A gDNA sequence. These SNPs corresponded to positions 1,629, 1,692, 1,721 and 1,725 of the wild-type PsCHLI-A gene sequence SEQ ID NO: 1 (FIG. 8).

### 3.3 Tasman Progenitor Line PW08-2308

The gDNA and cDNA sequences obtained for the Tasman green-yellow progenitor line were 100% identical to wild-type (PH11-0943) (SEQ ID NO:1 and SEQ ID NO: 2 (FIG. 8 and FIG. 9), respectively). Accordingly, this line contained the wild-type base (C) at the EMS-SNP position.

### 3.4 ‘Ted’ P. Somniferum Wild-Type Parent Line PW13-4611

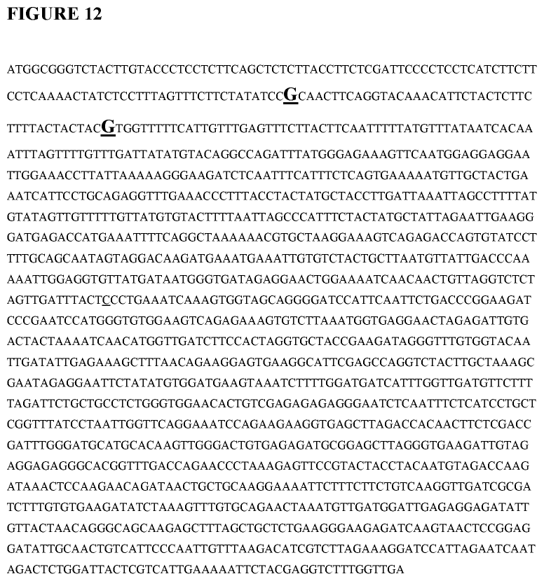

The ‘Ted’ PW13-4611 line has a thebaine chemotype in which thebaine is produced as the predominant alkaloid in poppy straw and latex of the plant and was similarly confirmed as containing the wild-type base at the EMS-SNP position. Two exonic and one intronic SNPs were identified within the PsCHLI-A gene coding region of this line (bases underlined in SEQ ID NO: 5; FIG. 12). One of the exonic SNPs was predicted to result in a non-synonymous amino acid change (S35A) whereas the second was a silent polymorphism (CTA to CTC codon change; both of which code for a leucine amino acid).

### 4. Gene Sequencing Results—TA Gene224147 (PsCHLI-B)

4.1 Wild-Type Line PH11-0943 and Comparison with PsCHLI-A

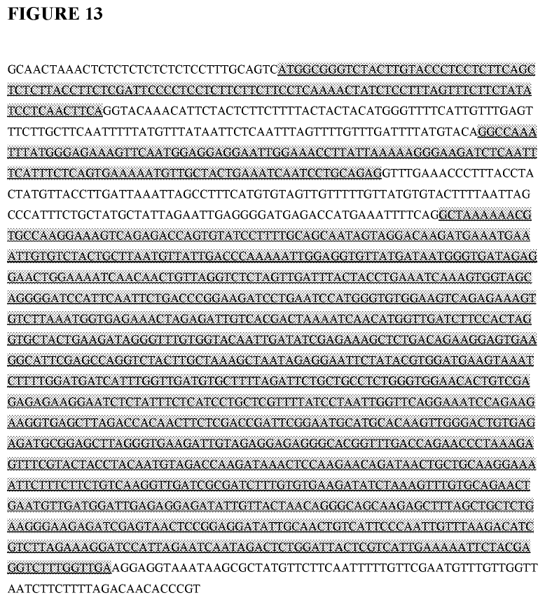

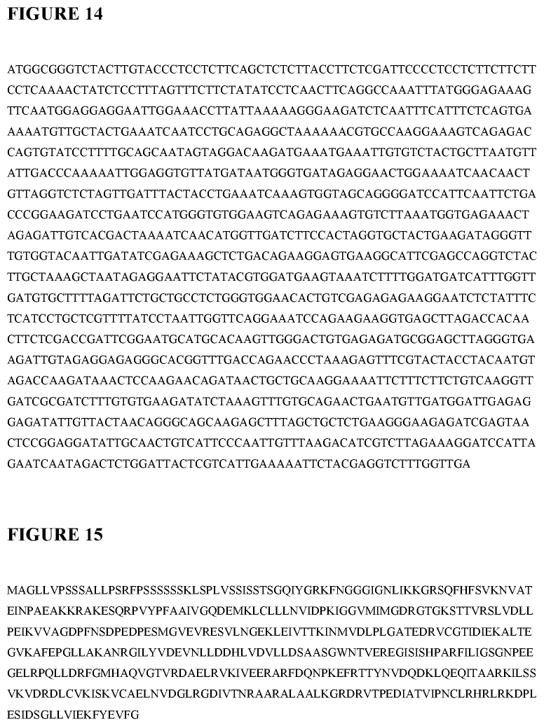

Sequencing and analysis of the PsCHLI-B gene generated a 1,641bp consensus gDNA sequence for the wild-type genotype PH11-0943 (SEQ ID NO: 6; FIG. 13). Apart from a single basepair indel occurring in the 3′ UTR, the gDNA PsCHLI-B sequence was 100% identical to the P. somniferum genome assembly sequence. Alignment of the PH11-0943 cDNA sequence obtained for PsCHLI-B (SEQ ID NO: 7; FIG. 14) against the gDNA sequence identified three exon regions within PsCHLI-B. As found for PsCHLI-A, the PsCHLI-B gene encoded a predicted 425 amino acid protein sequence (SEQ ID NO: 8; FIG. 15). PsCHLI-A and PsCHLI-B gene sequences shared very high sequence similarity, differing by only three amino acids at the protein level (I73N, E171K, Q384R; where the first amino acid listed is for PsCHLI-A). Based on this high similarity (99.3% protein similarity), the PsCHLI-B protein sequence also aligned well to the CHLI genes of other species (FIG. 6). Genetic features of the PsCHLI-B gDNA sequence are provided in Table 14.

TABLE 14
Gene features of the wild-type (PH11-0943)
TAgene224147 (PsCHLI-B) gDNA sequence

| SEQ ID NO: 6 | Coordinates | Length |
|---|---|---|
| (Feature) | (Nucleotide Nos.) | (Nucleotides) |
| 5′ UTR | 1-34 | 34 |
| Exon 1 | 35-145 | 111 |
| Intron 1 | 146-259 | 114 |
| Exon 2 | 260-376 | 117 |
| Intron 2 | 377-514 | 138 |
| Exon 3 | 515-1,564 | 1,050 |
| 3′ UTR | 1,564-1,641 | 77 |

### 4.2 Tasman EM4-0045 Light Green-Yellow Mutant

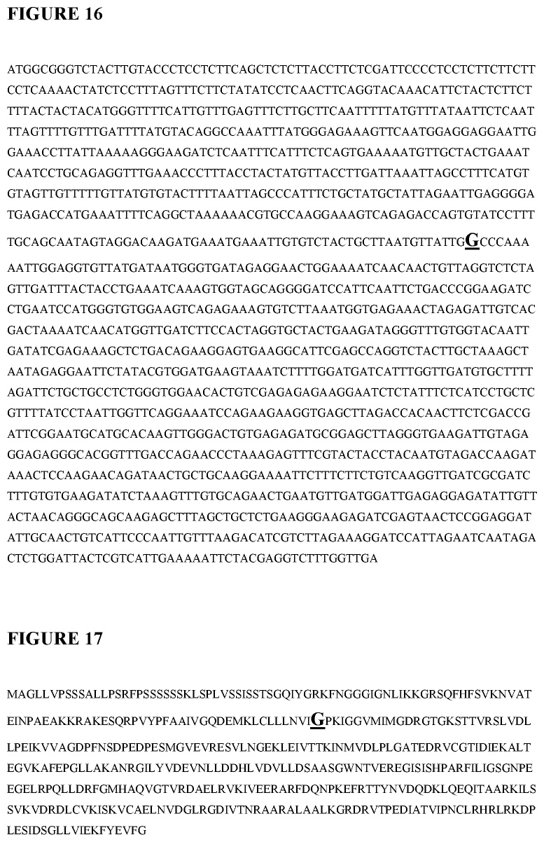

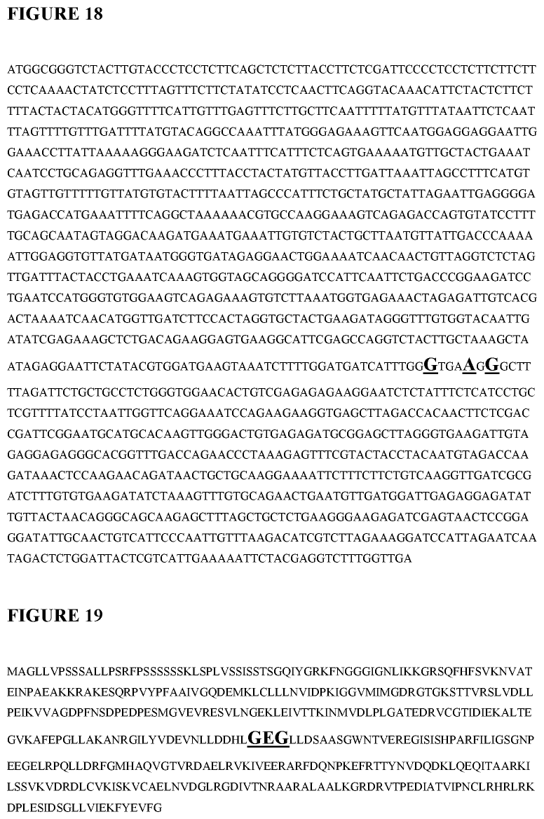

Sequencing identified three PsCHLI-B alleles within EM4-0045 line individuals. Allele 1 was 100% identical to the wild-type PsCHLI-B sequence (SEQ ID NO: 6; FIG. 13), whereas allele 2 contained a single SNP (A/G; see SEQ ID NO: 9; FIG. 16). This SNP corresponded to base pair position 615 of the wildtype gDNA sequence (i.e., SEQ ID NO: 6) and resulted in a predicted non-synonymous amino acid change within exon 3 of the PsCHLI-B EM4-0045 allele 2 protein sequence (SEQ ID NO: 10; FIG. 17). PsCHLI-B EM4-0045 allele 3 contained three SNPs, corresponding to basepair positions 993, 997 and 999, respectively, within exon 3 of the wild-type PsCHLI-B gDNA (SEQ ID NO: 11; FIG. 18). These SNPs resulted in three adjacent amino acid changes: V236G, D237E, V238G (see SEQ ID NO: 12; FIG. 19).

### 4.3 Tasman Progenitor Line PW08-2308 and Ted Line PW13-4611

All sequences obtained for Tasman PW08-2308 and Ted PW13-4611 individuals were 100% identical to the wild-type PsCHLI-B gDNA sequence.

### 4.4 Discussion

Two magnesium chelatase subunit I homologues where identified in the P. somniferum genome assembly sequence and were successfully isolated and sequenced in multiple P. somniferum genotypes. Characterization of wild-type (morphine line PH11-0943) P. somniferum genes revealed that both homologues, which have been designated PsCHLI-A and PsCHLI-B, encoded proteins 425 amino acids in length. The two poppy genes shared 99.3% sequence similarity at the protein level.

Sequencing confirmed an EMS-type SNP in PsCHLI-A of the light green-yellow mutant phenotype line EM4-0045. This SNP was unique to the EM4-0045 genotype, with the three lines examined in this study (all exhibiting the wild-type green-yellow phenotype) having the wild-type base (T) at the EMS-SNP position. The EMS SNP is predicted to cause a premature stop codon in the PsCHLI-A protein (Q328*). This mutation likely renders the PsCHLI-A protein non-functional in EM4-0045 and consequently, results in both disrupted chlorophyll biosynthesis and the light green-yellow phenotype of this line. PsCHLI-B sequences obtained for Tasman PW08-2308 and Ted PW13-4611 lines were identical to wild-type. Whilst allelic variation was detected in EM4-0045, the detected SNPs were predicted to result in missense amino acid substitutions only.

### EXAMPLE 9

### The PsCHLI-A Mutation Co-Segregates with Light Green-Yellow Leaf Colour, a Single-Gene Recessive Trait Associated with Altered Alkaloid Profile in P. Somniferum

A planthouse-grown F2 population was generated to examine the inheritance pattern of the light green-yellow colour trait and to evaluate whether this trait was associated with the EMS-SNP detected in PsCHLI-A (Q328*) of the light green-yellow mutant (EM4-0045). A second consideration of this cross was to illustrate that the light green-yellow trait is independent of chemotype and to examine whether this trait, when transferred to other P. somniferum chemo types, similarly results in improved alkaloid traits.

### 1. F2 Pedigree

Parents of the F2 pedigree were the Tasman (codeine chemotype) light green-yellow line EM4-0045 and the unrelated Ted (thebaine chemotype) line (PW13-4611) which had previously been shown to be pure-breeding for the typical wild-type, green-yellow leaf phenotype. Following the initial EM4-0045×PW13-4611 cross, the resulting F1 line (X15-0260) was self-pollinated to produce a F2 generation (PH16-2253 line).

### 2. Plant Growing Conditions

Eight pots of each parental line (EM4-0045 and PW13-4611), the F1 (X15-0260) and 50 pots of the F2 generation (PH16-2253) plants were grown in a planthouse at Tasmanian Alkaloids Pty Ltd (Westbury, Tasmania, Australia) during the 2017 winter season. Eight pots of the Tasman green-yellow line PW08-2308 from which the EM4-0045 line was derived was also planted as a control. Sowing occurred on 20 Apr. 2017 with seeds sown in 20 cm diameter pots. The potting mixture (per m³) comprised composted pine bark (800 L), sand (100 L), peat moss (100 L), dolomite lime (3 kg), hydrated lime (3 kg) and rock gypsum (1 kg). Once sown, seeds were covered with a thin (˜0.5 cm) layer of vermiculite and grown under an 18 hour light/8 hour dark photoperiod through use of supplemental lighting (high pressure sodium lamps; Horti Master greenPower, 600 W 400 v, E40). Automated climate control systems were used to maintain planthouse day and night temperatures at ˜20° C. and ˜15° C., respectively. Soil moisture content was maintained at 30-40% volume via an automated fertigation system which also supplied plant nutrients.

### 3. Assessment of the Colour Trait

Plants were visually assessed for leaf colour on 12 May 2017 (22 days after sowing) when seedlings were at the 4-6 true leaf stage (i.e., a foliage leaf of the plant as opposed to the cotyledonary leaves). All plants of both PW08-2308 and PW13-4611 parent lines exhibited wild-type green-yellow colour phenotypes. As expected, all plants of the light green-yellow line EM4-0045 had light green-yellow coloured leaves. All plants of the F1 line X15-0260 were green-yellow, suggesting that the light green-yellow colour phenotype is a recessive trait. Once assessed for colour phenotypes, pots of PW08-2308, PW13-4611, EM4-0045 and X15-0260 lines were thinned to six plants per pot.

The F2 population (50 pots) contained a total 411 plants. Owing to variable sowing rates amongst post the number of plants per pot ranged from between 3 to 21 (average number of plants per pot=8.22, mode=10). Following an initial assessment of the F2 population for colour phenotype on 12 May 2017 a second assessment was undertaken (16 May 2017) to double-check colour phenotypes within the segregating F2 population. Two abnormally small plants were scored as ‘unsure’ with all other plants being assessed as either green-yellow (305) or light green-yellow (104). The observed green-yellow and light green-yellow frequencies fitted a 3:1 segregation ratio (x² 0.0399, df=1, P=0.8416; where observed N=409 and expected green-yellow and light green-yellow plant frequencies were 306.75 and 102.25, respectively), confirming that the light green-yellow leaf trait of the EM4-0045 line is inherited as a single-gene recessive trait.

Pots of the F2 population were thinned to six plants per pot following the second assessment of leaf colour. This round of thinning was selective, with all light green-yellow plants being retained. In total, 287 plants remained post-thinning; comprising 104 light green-yellow plants and 183 green-yellow plants. Owing to the low sowing rate in some pots, not all F2 population pots contained six plants; e.g., a single pot contained three plants, two pots contained four plants per pot and six pots contained five plants per pot.

### 3. Plant Chemotype Classification

### 3.1 Chemotype Mutations

The ‘Ted’ (thebaine chemotype) PW13-4611 line used as a parent in the F2 cross is a ‘double chemotypic mutant’. This line contains two independent mutations which affect the benzylisoquinoline alkaloid pathway; the TOP1 (or Norman) mutation (Millgate et al., Morphine-pathway block in top1 poppies. Nature, Vol. 431, 413-414, 2004) and the O-demethylation mutation described in WO 2009/109012 (the ‘CODM’ mutation). As a result, the conversion of both thebaine to neopinone and thebaine to oripavine, respectively, is inhibited in Ted chemotypes and thereby plants containing these mutations accumulate thebaine (see Scheme 1 above). In contrast, the Tasman EM4-0045 line is a single ‘chemotypic’ mutant; containing the ‘CODM’ mutation only. A Ted (double chemotypic mutant)×Tasman (single chemotypic mutant) cross will therefore result in segregation of the recessive Norman mutation in F1 and F2 generations. The resulting F1 plants will be heterozygous for the Norman mutation and are predicted to have ‘Tasman’ (i.e., codeine) chemotypes and the F2 generation will segregate 3:1 for Tasman and Ted chemotypes.

### 3.2 Leaf Latex Analysis

Leaf latex alkaloid analysis was conducted 31 May 2017 (41 days after sowing) to confirm plant chemotype. To sample leaf latex, the uppermost part of the youngest fully developed leaf was removed. Latex droplets exuding from the intact severed leaf were then collected using the excised leaf tissue. The latex-covered excised tissue was then carefully placed into a sample plate filter well (Pall Acroprep GHP 0.2 μm 96-well). Up to 250 μL extractant solution (2% acetic acid 10% ethanol) was then added to each sample well and allowed to sit for 30 minutes. Samples were then filtered under vacuum into a 96-well collection plate for UPLC analysis as per the protocol described above.

Eighteen individual plants of each PW08-2308 (progenitor control), EM4-0045 (F2 parent), PW13-4611 (F2 parent) and X15-0260 (F1) were assayed with all plants found to have the expected chemotype (Table 15). In the F2 population, 102 light green-yellow plants (at this time one light green-yellow plant had died and another was damaged during leaf latex sampling and subsequently omitted) and 129 of the 183 green-yellow plants were assayed (Table 15). Consistent with the segregation of a single recessive gene (i. e. the TOP1/Norman mutation), a 3:1 ratio of Tasman (n=175) to Ted (n=56) chemotypes were obtained in the F2 generation (x² 0.0707, df=1, P=0.790; where observed N=231 and expected Tasman and Ted frequencies were 173.25 and 57.75, respectively).

TABLE 15
Chemotype results from leaf latex analysis

| Line | | Colour | | Chemotype | |
|---|---|---|---|---|---|
| (generation) | Chemotype* | phenotype* | n | Tasman | Ted |
| PW08-2308 | Tasman | Green-yellow | 18 | 18 | 0 |
| (Control; Tasman | | | | | |
| EM4-0045 | | | | | |
| progenitor line) | | | | | |
| EM4-0045 | Tasman | Light | 18 | 18 | 0 |
| (F2 cross parent) | | green-yellow | | | |
| PW13-4611 | Ted | Green-yellow | 18 | 0 | 18 |
| (F2 cross parent) | | | | | |
| X15-0260 (F1) | Tasman | Green-yellow | 18 | 18 | 0 |
| PH16-2253 (F2) | Segregating: | Green-yellow | 129^{a} | 104 | 25 |
| | Tasman | (¾**) | | | |
| | (¾**) | Light | 102^{b} | 71 | 31 |
| | and Ted | green-yellow | | | |
| | (¼**) | (¼**) | | | |

*Including predicted chemotype(s) and phenotypes.
**Expected segregation ratio.
^{a}129 out of 305 green-yellow plants were chemotyped.
^{b}102 out of 104 light green-yellow plants were chemotyped.

### 4. EMS-SNP Genotyping of the F2 Pedigree

High resolution melting (HRM) and Taqman genotyping assays were developed to test whether the EMS-SNP detected in the light green-yellow EM4-0045 line co-segregated with the light green-yellow trait in the F2 pedigree.

### 4.1 Genotyping Assays

For TaqMan assays two fluorescent probes were designed to be specific to each SNP allele. The probes were used in combination with PCR primers specific to the PsCHLI-A gene sequence (Table 16).

TABLE 16
TaqMan assay primer and probe sequences

| | | SEQ |
|---|---|---|
| | | ID |
| Primer/probe | Sequence | NO: |
| Forward primer | ACGGTTTGACCAGAACCCTAAAGAG | 21 |
| Reverse primer | TCGCGATCAACCTTGACAGAAG | 22 |
| Probe 1 | ACCAAGATAAACTCTAAGAACA | 23 |
| (mutant ‘T’ SNP) | | |
| Probe 2 | ACCAAGATAAACTCCAAGAAC | 24 |
| (WT ‘C’ SNP) | | |

Each reaction contained 1×TaqMan mastermix (enzyme and buffer; Applied Biosystems), 750 nM forward primer, 750 nm reverse primer, 250 nM probe 1, 250 nM probe 2 and 10 ng DNA. Reactions were performed in a Roche Lightcycler® 480 (Roche Diagnostics GmbH, Mannheim, Germany) and incubated through 1 cycle of 95° C. for 4 minutes, 45 cycles of 95° C. at 5 seconds, 60° C. for 40 seconds and 40° C. for 30 seconds. Endpoint readings of the fluorescence from 6-carboxyfluorescein-FAM dye (C SNP) and CAL Fluor Orange 560 dye (T SNP), generated during the PCR amplification, were plotted using Lightcycler 480® software V1.5 (Endpoint Genotyping module). Plots were visually inspected to ensure genotype calls were correct.

For HRM studies forward (5′-CGGTTTGACCAGAACCCTAA-3′) (SEQ ID NO: 25) and reverse (5′-AAAGAATTTTCCTTGCAGCAG-3′) (SEW ID NO:26) primers were designed to target 93bp of the PsCHLI-A gene spanning the C to T SNP. PCR amplification and HRM analysis were performed in a Roche Lightcycler® 480. PCR amplification utilised 1× mastermix (enzyme/buffer), 2.5 mM MgCl₂, 200 nM forward primer, 200 nm reverse primer and 10 ng DNA. The PCR reaction had an initial denaturation step of 95° C. for 5 minutes, followed by 45 cycles of 10 seconds at 95° C. (denaturation), 20 seconds at 57° C. (annealing) and 30 seconds at 72° C. (extension). The PCR amplification was then followed by heteroduplex formation by heating at 95° C. for 1 minute and subsequent cooling at 40° C. for 1 minute. High resolution melting analysis was performed immediately afterwards by increasing the temperature in two sequential steps from 40° C. to 65° C. (ramp rate of 2.5° C.^{-sec}) and then to 95° C. (ramp rate of 4.8° C.^{-sec}). The HRM data was analysed using Lightcycler 480® Gene Scanning software.

### 4.2 F2 Pedigree Analysis

One hundred and twenty-five individuals were genotyped for the EMS-SNP. These plants were selected based on phenotype and chemotype and included 20 individuals each of the four chemotypic-phenotypic classes segregating in the F2 population (Table 17). The genotyping analysis also included 15 individuals of line PW08-2308 as controls (also see Table 17).

Sample DNA was isolated as described above in Example 8 and both HRM and Taqman assays were performed as described above. Both assays clearly discriminated homozygous wild-type, heterozygous, and homozygous mutant genotypes and retuned identical genotype calls for all individuals. All PW08-2308 (control) and Ted F2 parent (PW13-4611) individuals were homozygous for the wild-type PsCHLI-A allele (CC genotype; Table); being concordant with the green-yellow phenotype of these lines. All EM4-0045 light green-yellow individuals (n=15) were homozygous for the EMS-SNP allele (TT genotype) and as expected all F1 individuals were heterozygous (CT genotype); further confirming the recessive nature of the PsCHLI-A mutant allele.

In the F2 generation, all green-yellow plants (n=40) had either ‘CC’ or ‘CT’ genotypes. For light green-yellow plants, 38 out of 40 individuals were genotyped as being homozygous mutants (TT genotype; Table 17). F2 generation plants 830-1 (Tasman light green-yellow) and 856-2 (Ted light green-yellow) were genotyped as heterozygous and homozygous wild-type genotypes, respectively; these genotypes disagreeing with the phenotypic assessments of these plants. To investigate this discrepancy, seed obtained from self-pollinated 830-1 (line PW17-2576) and 856-2 (line PW17-2606) plants were sown to examine the phenotype of F3 progeny (note: F2 plants had completed their life cycle and had become desiccated by the time genotyping results became available, thus, not being possible to re-check colour assessments at that time). Unfortunately, the seed obtained from plant 830-1 was poor and failed to germinate. Conversely, the sown seed of plant 856-2 successfully germinated and all 50 seedlings were observed to have a green-yellow colour phenotype. This finding confirmed that plant 856-2 had been incorrectly phenotyped for colour and that its' genotype was indeed correct. It seems highly probable that the incongruent result for plant 830-1 can similarly be explained by either phenotypic or sampling error.

In summary, phenotypic and genotypic assessment of the EM4-0045×PW13-4611 F2 pedigree confirmed that the CHLI-A EMS mutation co-segregates with the light green-yellow colour trait and that this trait is inherited as a single gene recessive trait.

TABLE 17
EMS-SNP genotyping results. Genotypes obtained from HRM/Taqman
assays are shown for each line and class of the F2 pedigree

| | Class | | Genotype class | | |
|---|---|---|---|---|---|
| | (Chemotype | | Homozygous | | Homozygous |
| Line | and | | wild-type | Heterozygous | mutant |
| (Generation) | Phenotype) | n | CC | CT | TT |
| PW08-2308 | Tasman | 15 | 15 | — | — |
| (Control; Tasman | Green-yellow | | | | |
| EM4-0045 | | | | | |
| progenitor line) | | | | | |
| EM4-0045 | Tasman | 15 | — | — | 15 |
| (F2 cross parent) | Light green- | | | | |
| | yellow | | | | |
| PW13-4611 | Ted | 15 | 15 | — | — |
| (F2 cross parent) | Green-yellow | | | | |
| X15-0260 | Tasman | 15 | — | 15 | — |
| (F1) | Green-yellow | | | | |
| PH16-2253 (F2) | Tasman | 20 | 5 | 15 | — |
| | Green-yellow | | | | |
| | Tasman | 20 | — | 1^{a} | 19 |
| | Light green- | | | | |
| | yellow | | | | |
| | Ted | 20 | 5 | 15 | |
| | Green-yellow | | | | |
| | Ted | 20 | 1^{b} | — | 19 |
| | Light green- | | | | |
| | yellow | | | | |

| | |
|---|---|
| Total | 140 |

^{a}Unconfirmed and
^{b}confirmed phenotyping/sampling error (see above)

### 5. Capsule Alkaloid Content

Plants of the F2 pedigree were grown to maturity and allowed to dry naturally. Very few plants developed secondary branches/capsules with nearly all plants within the population being single-stemmed plants. To prevent outcrossing with neighbouring poppy lines, all plants were ‘bagged selfed’. This involved placing a small paper bag over the unopened flower bud when in the ‘late hook’ or ‘upright bud’ stage and securing the bag with a plastic tie. This bagging method prevents pollen dispersal and outcrossing of the bagged plant yet still allows for self-pollination to occur.

Capsule alkaloid assessments were made on the same 140 plants which were genotyped. Seven plants were omitted from the capsule alkaloid analysis due to the plants and/or capsules being damaged during the experiment (five plants) or phenotype-genotype discrepancies. Following plant desiccation, capsules were abscised at the position directly below the peduncle. Seeds were removed before oven-drying capsules at 65° C. for 12 hours to remove any residual moisture content and to standardize moisture content across samples. Individual capsules were then ground to a consistent particle size using an electric coffee/spice grinder. The average weight of dried ground capsules was 1.13 g (range 0.34 g to 2.55 g). The whole, ground capsule material was used for individual capsule alkaloid extractions. For seven samples where the oven-dried capsule weight exceeded two grams, a 2.00 g subsample was used for alkaloid extraction.

Capsule alkaloids were extracted in a 2% acetic acid and 10% ethanol solution using a 2.00 g tissue to 40 mL extractant ratio, which was scaled accordingly for the variable capsule weights. Samples were shaken for 90 minutes on a Ratek orbital shaker before transferring a 240 μL aliquot of each sample to a 96-well Pall filter plate (GHP 0.2 μm). Samples were then filtered under vacuum into a 96-well collection plate for UPLC analysis utilising the protocol described in Example 2 above.

### 6. Capsule Alkaloid Results

Capsular alkaloid content was quantified for green-house grown F2 pedigree plants and the Tasman PW08-2308 progenitor line. The average codeine and thebaine contents for each of the lines on a dry weight basis (% DW) are shown in Table 18, as well as the generations and/or classes examined. As can be seen, the light green-yellow mutant line EM4-0045 contained a greater mean codeine content (3.838% DW vs. 3.669% DW; 4.6% increase) and substantially lower mean thebaine to codeine ratio (0.013 vs. 0.061) relative to its' progenitor line PW08-2308. In the F2 generation, Tasman plants being homozygous for the mutant PsCHLI-A allele and exhibiting the light green-yellow phenotype were similarly found to contain a higher mean codeine content (3.354% DW vs. 3.212% DW; 4.4% increase) and lower mean thebaine to codeine ratio (0.168 vs. 0.243) relative to Tasman plants exhibiting the wild-type green-yellow colour phenotype.

Ted plants exhibiting the light green-yellow colour phenotype were also found to have improved alkaloid content in the F2 pedigree. For example, the mean thebaine content of F2 generation light green-yellow Ted plants was 2.896% DW in comparison to 2.811% DW in F2 generation Ted plants having the wild-type green-yellow colour phenotype; equivalent to a mean 3% thebaine increase (Table 18).

TABLE 18
Capsule alkaloid results obtained from
the greenhouse-grown F2 population

| | | | Mean alkaloid content* (% DW) | | | |
|---|---|---|---|---|---|---|
| Line | Class (Chemotype | | | | T/C | Total |
| (Generation) | and phenotype) | n | C | T | ratio | CT |
| PW08-2308 | Tasman | 15 | 3.669 | 0.243 | 0.061 | 3.912 |
| (Control; | Green-yellow | | | | | |
| Tasman | | | | | | |
| EM4-0045 | | | | | | |
| progenitor | | | | | | |
| line) | | | | | | |
| EM4-0045 | Tasman | 15 | 3.838 | 0.052 | 0.013 | 3.889 |
| (F2 cross | Light | | | | | |
| parent) | green-yellow | | | | | |
| PW13-4611 | Ted | 14 | 0.007 | 2.924 | n/a | 2.930 |
| (F2 cross | Green-yellow | | | | | |
| parent) | | | | | | |
| X15-0260 | Tasman | 15 | 2.597 | 0.442 | 0.169 | 3.040 |
| (F1) | Green-yellow | | | | | |
| PH16-2253 | Tasman | 20 | 3.212 | 0.727 | 0.243 | 3.939 |
| (F2) | Green-yellow | | | | | |
| | Tasman | 19 | 3.354 | 0.528 | 0.168 | 3.882 |
| | Light | | | | | |
| | green-yellow | | | | | |
| | Ted | 0 | 0.004 | 2.811 | n/a | 2.815 |
| | Green-yellow | | | | | |
| | Ted | 5 | 0.003 | 2.896 | n/a | 2.900 |
| | Light | | | | | |
| | green-yellow | | | | | |

| | |
|---|---|
| Total | 33 |

*Values are percent dry weight (% DW); C = codeine, T = thebaine. Thebaine to codeine ratios (T/C) are shown for Tasman plants only.

In summary, phenotypic, genotypic and segregation analysis in a Ted×Tasman F2 generation has shown the EMS-SNP detected within the PsCHLI-A gene co-segregates with the light green-yellow leaf colour phenotype, that this colour trait is controlled by a single gene and that the light green-yellow allele is recessive to the wild-type allele responsible for the normal green-yellow leaf colour phenotype.

The PsCHLI-A mutation was successfully transferred from the Tasman EM4-0045 into a Ted cultivar (PW13-4611), showing that the PsCHLI-A mutation is chemotype independent. Results also show that the light green-yellow leaf colour trait is associated with beneficial alkaloid traits in poppy straw on a dry weight basis of the straw in two chemotypic backgrounds; namely an increase in thebaine by weight in the poppy straw of Ted and an increase in codeine content by weight, a decrease in thebaine content by weight, and an overall decrease in the ratio of thebaine to codeine (T/C) by weight in the poppy straw of Tasman.

### EXAMPLE 10

### The PsCHLI-A Mutation Provides Improved Alkaloid Traits in a (Wild-Type) Plant of P. Somniferum with a Morphine Chemotype

Further plant breeding was undertaken to introduce disease-resistant traits from a low alkaloid disease-resistant morphine line (PW10-1325) into the light green-yellow mutant Tasman EM4-0045 genotype. A by-product of the crossing strategy employed were light green-yellow plants having morphine chemotypes. These morphine plants were examined to investigate the effect of the PsCHLI-A mutation (Q328*) on the wild-type morphine chemotype.

### 1. Pedigree, Plant Growing Conditions and Plant Leaf Colour Assessment

Several F1 plants derived from the initial morphine PW10-1325×Tasman EM4-0045 cross were grown and self-pollinated. A small number of resulting F2 families (eight) were then grown under greenhouse conditions and assessed for plant leaf colour and alkaloid traits. Four pots of each F2 line were sown on 25 Oct. 2017 and grown in a planthouse at Tasmanian Alkaloids Pty Ltd (Westbury, Tasmania, Australia) during the 2017-18 summer season. Plant growing conditions were as described in Example 8. Plants were visually assessed for leaf colour at the ˜6 leaf stage on 1 Dec. 2017 and again on 8 Dec. 2017 to confirm plant leaf colour. All F2 families segregated for wild-type green-yellow and mutant light green-yellow leaf colour phenotypes. Pots were subsequently thinned to six plants per pot following this colour assessment.

### 2. Capsule Latex Alkaloid Analysis

Capsule latex analysis was conducted on 9 Feb. 2018 to identify F2 plants having morphine chemotypes (each F2 family segregated for Tasman (i.e., codeine) and morphine chemotypes). Capsule latex was obtained by removing a single stigmatic ray from the capsule of each near-desiccated plant. A small amount of capsule exudate (i.e., latex) was then collected on the removed stigmatic ray and transferred to a sample plate filter well (Pall Acroprep GHP 0.2 μm 96-well). Up to 250 μL extractant solution (2% acetic acid 10% ethanol) was then added to each sample well and allowed to sit for 30 minutes before filtering and undertaking UPLC analysis as describedabove. Of the 72 plants analysed, 54 morphine and 18 Tasman chemotypes were obtained; being a near-perfect 3:1 segregation ratio (x² 0.0740, df=1, P=0.782; where observed N=72 and expected morphine and Tasman frequencies were 54 and 18, respectively).

### 3. Whole Capsule Alkaloid Analysis

Combining chemotype and plant leaf colour data, 22 morphine green-yellow (i.e., wild-type leaf colour) and 13 morphine light green-yellow (i.e., PsCHLI-A mutant leaf colour) plants were selected for whole capsule alkaloid analysis. Following plant desiccation, capsules were abscised at the position directly below the peduncle. Seeds were then removed before grinding individual capsules to a consistent particle size using an electric coffee/spice grinder. The average weight of dried ground capsules was 1.21 g (range 0.5 g to 2.37 g). As described in Example 9, the whole, ground capsule material was used for individual capsule alkaloid extractions. For one sample where the oven-dried capsule weight exceeded two grams, a 2.00 g subsample was used for alkaloid extraction. Capsule alkaloids were then extracted using a 2% acetic acid and 10% ethanol solution and a 2.00 g tissue to 40 mL extractant ratio as described previously.

Table 19 presents the capsule alkaloid results obtained for light green-yellow and green-yellow morphine plants. As seen, the light green-yellow plants contained substantially greater total alkaloid content (1.935% DW vs 1.615% DW), equating to an approximate 20% increase over plants exhibiting the wild-type green-yellow colour phenotype. This increase in total alkaloid was driven by increases in both morphine and codeine content in the light green-yellow plants. Interestingly, the light green-yellow plants contained less thebaine, both on a % DW basis and as a percentage of total MOCT, and greater codeine as a percentage of total MOCT, relative to green-yellow plants. Thus, the light green-yellow morphine plants contained a markedly lower T/C ratio (0.217) in comparison to green-yellow morphine chemotype plants (T/C ratio 0.538). This finding was consistent with the Tasman (codeine) chemotype results described in Example 9, in which the PsCHLI-A gene mutation results in a reduced T/C ratio in light green-yellow plants.

TABLE 19
Capsule alkaloid results obtained from
greenhouse-grown morphine plants

| | | | | | | Total |
|---|---|---|---|---|---|---|
| | | Percent dry weight (percent of total MOCT)* | | | | MOCT |
| Class | n | Morphine | Oripavine | Codeine | Thebaine | % DW |
| Morphine | 22 | 1.178 | 0.017 | 0.273 | 0.147 | 1.615 |
| green-yellow | | (74.3) | (1.1) | (16.1) | (8.6) | |
| Morphine | 13 | 1.397 | 0.017 | 0.428 | 0.093 | 1.935 |
| light green- | | (72.9) | (0.9) | (21.6) | (4.6) | |
| yellow | | | | | | |

*Alkaloid values are shown on a percent dry weight basis (% DW).

### EXAMPLE 11

### Alkaloid Content (% w/w) of EM4-0045 and EM4-0019 Mutant Plant Lines

New lines arising from Tasmanian Alkaloids Pty Ltd's Poppy Breeding Program are routinely tested in field trials to assess their performance across a range of growing regions, environments and seasons. These field trials can take various forms, from single-location, single-replicate, single-row-screening trials (as described above in Example 2 and 3) through to multiple-location, multiple-replicate-plot trials (as described above in Examples 4 and 7). Regardless of the trial design, field dried capsules from each trial plot/replicate are harvested at maturity, threshed to remove seed, and the remaining ‘straw’ sample is ground, and alkaloids are extracted and analysed as described in Example 2. Alkaloid data from these trials is then expressed as % w/w in field-dried straw.

Examination of such trial data derived from a range of locations and seasons enables an understanding of Genotype x Environment interactions in the expression of the desirable alkaloid traits and also, an insight into the genetic potential of these new lines. An examination of all field-trial raw data (alkaloid contents in samples from each replicate plot in every trial) from season 2013/14 through to season 2018/19 reveals some particularly high alkaloid contents in both EM4-0045 (Table 20) and the EM4-0019 (Table 21) plants.

For instance, a codeine content of 5.79% was recorded in the light-green-yellow leaf mutant line EM4-0045 whilst a T/C ratio of 0.005 was also observed in these trials for that plant line as shown in Table 20 in which the alkaloid data is expressed as % w/w in field dried, hand harvested straw samples.

TABLE 20
Results for a selection of field trial plots/replicates for plant line EM4-0045

| | | | | block/ | | Codeine | Thebaine | T/C |
|---|---|---|---|---|---|---|---|---|
| | Trial Name | Season | Location | replicate | plot | (% w/w) | (% w/w) | ratio |
| A | TASRCVT18 | 2017/18 | Rocky Cape | 2 | 593 | 5.79 | 0.21 | 0.036 |
| | TASSVT16 | 2015/16 | Sassafras | 1 | 294 | 4.77 | 0.45 | 0.094 |
| | TASSVT16 | 2015/16 | Sassafras | 2 | 319 | 4.74 | 0.47 | 0.100 |
| | TASTCVT18 | 2017/18 | Table Cape | 1 | 437 | 4.66 | 0.23 | 0.049 |
| | TASNSVT17 | 2016/17 | Forest | 3 | 428 | 4.65 | 0.28 | 0.060 |
| | MINT14r | 2013/14 | Moriarty | 1 | 14 | 4.60 | 0.40 | 0.087 |
| B | DM15 | 2014/15 | Sassafras | 3 | 212 | 4.19 | 0.02 | 0.005 |
| | DM15 | 2014/15 | Sassafras | 2 | 140 | 4.28 | 0.03 | 0.007 |
| | TASCVT15 | 2013/14 | Cressy | 1 | 14 | 4.39 | 0.06 | 0.013 |
| | TASCVT15 | 2013/14 | Cressy | 2 | 21 | 4.35 | 0.06 | 0.014 |
| | TASPVT18 | 2017/18 | Penguin | 3 | 740 | 4.22 | 0.07 | 0.016 |
| | TASRCVT18 | 2017/18 | Rocky Cape | 1 | 557 | 4.57 | 0.08 | 0.018 |

Furthermore, in the lime-green leaf mutant line EM4-0019, thebaine content over 5% were relatively common with a 5.88% thebaine content in straw being observed in these trials, see Table 21 in which the alkaloid data is again expressed as % w/w in field-dried, hand harvested straw samples.

TABLE 21
A selection of field trial replicate results for plant line EM4-0019

| | | | Block/ | | Thebaine |
|---|---|---|---|---|---|
| Trial Name | Season | Location | Replicate | Plot | (% w/w) |
| TEDSVT16 | 2015/16 | Sassafras | 3 | 422 | 5.88 |
| TEDHINT16 | 2015/16 | Hagley | 2 | 1052 | 5.47 |
| TEDSVT16 | 2015/16 | Sassafras | 2 | 388 | 5.45 |
| TEDHINT16 | 2015/16 | Hagley | 1 | 1015 | 5.42 |
| TEDLVT16 | 2015/16 | Latrobe | 3 | 537 | 5.38 |
| TEDLVT16 | 2015/16 | Latrobe | 1 | 446 | 5.36 |
| TEDLVT16 | 2015/16 | Latrobe | 2 | 503 | 5.36 |
| TEDHVT16 | 2015/16 | Hagley | 1 | 242 | 5.26 |
| TEDHVT16 | 2015/16 | Hagley | 2 | 285 | 5.24 |
| TEDSVT16 | 2015/16 | Sassafras | 1 | 339 | 5.19 |
| TEDHINT16 | 2015/16 | Hagley | 3 | 1163 | 5.11 |
| TEDPVT15 | 2014/15 | Perth | 2 | 406 | 5.11 |
| TEDFVT16 | 2015/16 | Forth | 1 | 8 | 5.09 |
| TEDPVT15 | 2014/15 | Perth | 1 | 371 | 5.05 |
| TEDPVT15 | 2014/15 | Perth | 3 | 413 | 5.03 |
| TEDHVT16 | 2015/16 | Hagley | 3 | 320 | 5.01 |
| MVT14 | 2013/14 | Moriarty | 2 | 126 | 4.81 |
| TEDMVT19 | 2018/19 | Moriarty | 3 | 121 | 4.73 |

While the foregoing specification teaches the principles of the present invention, with examples provided for the purpose of illustration, it will be understood that the practice of the invention encompasses variations, adaptations and/or modifications as come within the scope of the following claims and their equivalents.

1. A plant of Papaver somniferum modified to have reduced expression or activity of magnesium chelatase relative to a wild-type P. somniferum whereby the plant upon the harvesting of its poppy capsules will yield poppy straw having an altered alkaloid profile compared to the wild-type plant.

2. The plant according to claim 1, wherein the reduced expression or activity of magnesium chelatase is associated with a single gene of the plant.

3. The plant according to claim 2, wherein the gene manifests in a trait for a light leaf colour phenotype of the plant compared to the wild-type P. somniferum.

4. The plant according to claim 2 or 3, wherein the gene is a recessive gene.

5. The plant according to any one of claims 2 to 4, wherein the gene encodes a magnesium chelatase subunit and expression of the gene is reduced in the plant, or the gene is expressed and encodes a mutant form of the magnesium chelatase subunit, whereby magnesium chelatase activity in the plant is thereby reduced.

6. The plant according to claim 5, wherein the gene encodes a mutant form of the subunit.

7. The plant according to claim 5, wherein the subunit is a magnesium chelatase I-A (CHLI-A) subunit.

8. The plant according to claim 7, wherein the CHLI-A subunit is truncated compared to the wild-type form of the subunit.

9. The plant according to claim 8, wherein the CHLI-A subunit has anucleic acid sequence as set forth in SEQ ID NO: 2.

10. The plant according to claim 8 or 9, wherein the wild-type CHLI-A subunit has an amino acid sequence as set forth in SEQ ID NO: 3.

11. The plant according to any one of claims 7 to 10, wherein the plant has a further gene encoding a magnesium chelatase I-B (CHLI-B) subunit which is expressed in the plant.

12. The plant according to claim 11, wherein the CHLI-B subunit has greater than 99% amino acid sequence identity with SEQ ID NO: 3.

13. The plant according to claim 11 or 12, wherein the CHLI-B subunit has an amino acid sequence as set forth in SEQ ID NO: 8.

14. The plant according to claim 12 or 13, wherein the CHLI-B subunit of the plant has 100% amino acid sequence identity with the wild-type form of CHLI-B subunit.

15. The plant according to any one of claims 1 to 14, wherein the proportion of one or more alkaloids selected from thebaine, morphine, oripavine is increased in the poppy straw of the plant compared to the wild-type P. somniferum.

16. The plant according to claim 15, wherein the proportion of thebaine is increased in the poppy straw.

17. The plant according to any one of claims 1 to 16, wherein the poppy straw of the plant has an increased thebaine content compared to the wild-type P. somniferum.

18. The plant according to any one of claims 1 to 17, wherein the plant has a thebaine chemotype in which thebaine is the predominant alkaloid in the poppy straw.

19. A method for providing a plant of P. somniferum having an altered alkaloid profile, comprising:
a) exposing at least one poppy seed of a Papaver somniferum parent plant to a mutagenizing agent;
b) growing the at least one poppy seed exposed to the mutagenizing agent to produce one or more further plants, optionally through one or more self-fertilised generations; and
c) providing a plant from the one or more plants which is identified to have reduced expression or activity of magnesium chelatase activity relative to the parent plant whereby upon the harvesting of poppy capsules of the identified plant that plant yields a poppy straw having an altered alkaloid profile compared to the P. somniferum parent plant.

20. The method according to claim 19, wherein the identification of the plant having the altered alkaloid profile comprises screening the plant for reduced expression or activity of magnesium chelatase.

21. The method according to claim 20, wherein the screening comprises screening for defective magnesium chelatase in the plant.

22. The method according to claim 20, comprising screening for a defective magnesium chelatase subunit in the plant.

23. The method according to claim 20 or 21, wherein the screening comprises screening for a mutant gene encoding a magnesium chelatase subunit or for a mutation, wherein the gene or mutation is associated with reduced expression or activity of magnesium chelatase.

24. The method according to claim 23, wherein the plant has reduced expression of the gene.

25. The method according to claim 23, wherein the gene is expressed in the plant and the subunit encoded by the gene is defective whereby magnesium chelatase comprising the subunit has reduced activity.

26. The method according to any one of claims 23 to 25, wherein the subunit is a magnesium chelatase I-A (CHLI-A) subunit.

27. A method for providing a modified plant of Papaver somniferum with an altered alkaloid profile, comprising reducing expression or activity of magnesium chelatase in a plant of P. somniferum whereby upon the harvesting of its poppy capsules, the plant yields a poppy straw having an altered alkaloid profile.

28. The method according to claim 27, comprising reducing expression of magnesium chelatase in the plant.

29. The method according to claim 28, wherein the reduction in expression of magnesium chelatase in the plant comprises reducing expression of a magnesium chelatase subunit in the plant.

30. The method according to claim 27, comprising introducing at least one mutation into a gene encoding for a magnesium chelatase subunit whereby magnesium chelatase in the modified plant has reduced activity.

31. The method according to claim 30, whereby the wild-type form of the gene into which the mutation is introduced is set forth in SEQ ID NO: 1.

32. The method according to claim 29 or 30, wherein the subunit is a magnesium chelatase I-A (CHLI-A) subunit.

33. The method according to any one of claims 27 to 32, wherein the modified plant has a gene encoding a magnesium chelatase I-A (CHLI-A) subunit having an amino acid sequence as set forth in SEQ ID NO: 3.

34. A method for providing a plant of Papaver somniferum with an altered alkaloid profile, comprising:
cross-pollinating a first parent plant of Papaver somniferum modified to have reduced expression or activity of magnesium chelatase in the plant with a second parent plant of P. somniferum of a different chemotype compared to the first parent plant, to produce a first generation descendent P. somniferum plant;
self-pollinating the first generation plant to produce a second generation descendent P. somniferum plant having reduced expression or activity of magnesium chelatase wherein the second generation plant upon the harvesting of its poppy capsules yields poppy straw having the altered alkaloid profile compared.

35. The method according to claim 34, further comprising producing one or more further generations of descendent plants from the second generation plant, wherein the altered alkaloid profile is exhibited by the one or more further generations of descendent plants.

36. The method of claim 34 or 35, wherein the reduced expression or activity of magnesium chelatase in the first parent plant is associated with a single gene of the first parent plant.

37. The method according to claim 36, wherein the gene manifests in a trait for a light leaf colour phenotype of the plant compared to a corresponding wild-type P. somniferum.

38. The method according to claim 36 or 37, wherein the gene is a recessive gene.

39. The method according to any one of claims 36 to 38, wherein the gene encodes a magnesium chelatase subunit and expression of the gene is reduced in the first parent plant, or the gene is expressed in the first parent plant and encodes a mutant form of the magnesium chelatase subunit, whereby magnesium chelatase activity in the first parent plant is thereby reduced.

40. The method according to claim 39, wherein the gene encodes a mutant form of the subunit.

41. The method according to claim 39 or 40, wherein the subunit is truncated compared to the wild-type form of the subunit.

42. The method according to any one of claims 39 to 41, wherein the subunit is a magnesium chelatase I-A (CHLI-A) subunit.

43. The method according to any one of claims 34 to 42, wherein the first parent plant has a thebaine chemotype in which thebaine is the predominant alkaloid or a morphine chemotype in which morphine is the predominant alkaloid.

44. The method according to any one of claims 34 to 42, wherein the first parent plant has a codeine chemotype in which codeine is the predominant alkaloid.

45. The method according to any one of claims 34 to 42, wherein the second parent plant has a thebaine chemotype in which thebaine is the predominant alkaloid.

46. A method for increasing the content of an alkaloid in a plant of Papaver somniferum, the method comprising modifying the plant to reduce expression or activity of magnesium chelatase in the plant.

47. The method according to claim 46 wherein the content of the alkaloid is increased relative to one or more other alkaloids in the plant.

48. The method according to claim 46 or 47, wherein the alkaloid is thebaine.

49. The method according to any one of claims 46 to 48, wherein plant has a thebaine chemotype in which the predominant alkaloid is thebaine.

50. A method for identifying a plant of Papaver somniferum having an altered alkaloid profile, comprising screening the plant for reduced expression or activity of magnesium chelatase whereby, upon the harvesting of poppy capsules of the plant, the plant yields a poppy straw having the altered alkaloid profile.

51. The method according to claim 50, wherein the screening comprises screening for defective magnesium chelatase in the plant.

52. The method according to claim 51, comprising screening for a defective magnesium chelatase I-A (CHLI-A) subunit in the plant.

53. The method according to claim 51, wherein the screening comprises screening for a mutant gene encoding for a magnesium chelatase subunit.

54. The method according to claim 53, wherein the plant has reduced expression of the gene.

55. The method according to claim 53 or 54, wherein the gene is expressed in the plant and the encoded subunit is defective whereby magnesium chelatase comprising the subunit has reduced activity.

56. The method according to any one of claims 53 to 55, wherein the subunit is a magnesium chelatase I-A (CHLI-A) subunit.

57. A method for providing a poppy straw, comprising:
obtaining the poppy straw from poppy capsules harvested from a plant as defined in any one of claims 1 to 18 or from a plant provided by a method as defined in any one of claims 19 to 49.

58. The method according to claim 57, wherein the obtaining of the poppy straw comprises threshing the poppy capsules to remove seeds.

59. A method for providing an opium, comprising:
collecting latex from immature poppy capsules of a plant as defined in any one of claims 1 to 18 or from immature poppy capsules of a plant provided by a method as defined in any one of claims 19 to 49; and
drying the latex to provide the opium.

60. A poppy straw harvested from poppy capsules of a plant as defined in any one of claims 1 to 18 or from poppy capsules of a plant provided by a method as defined in any one of claims 19 to 49.

61. A latex for the extraction of one or more alkaloids, the latex being a latex from immature poppy capsules of a plant as defined in any one of claims 1 to 18 or from immature poppy capsules of a plant provided by a method as defined in any one of claims 19 to 49.

62. An opium obtained by drying a latex from immature poppy capsules of a plant as defined in any one of claims 1 to 18 or from immature poppy capsules of a plant provided by a method as defined in any one of claims 19 to 49.

63. A concentrate of poppy straw being a concentrate of the poppy straw of claim 60.

64. An alkaloid extracted from the poppy straw of claim 60, the latex of claim 61, the opium of claim 62, or the concentrate of poppy straw of claim 63.

65. Seed of a plant as defined in any one of claims 1 to 18 or a plant provided by a method as defined in any one of claims 19 to 49.

66. A plant cell or plant root from a plant as defined in any one or claims 1 to 18 or from a plant provided by a method as defined in any one of claims 19 to 49.

67. A descendent plant of a plant as defined in any one of claims 1 to 18, wherein the descendent plant exhibits the reduced expression or activity of magnesium chelatase, or is heterozygous for a modified gene associated with the reduced expression or activity of magnesium chelatase.

68. A descendent plant of a plant provided by a method as defined in any one of claims 19 to 49, wherein the descendent plant exhibits the reduced expression or activity of magnesium chelatase, or is heterozygous for a modified gene associated with the reduced expression or activity of magnesium chelatase.
